(19) United States
(12) Patent Application Publication
(10) Pub. No.: US 2024/0307524 A1
(43) Pub. Date: Sep. 19, 2024

(54) CORONAVIRUS VACCINES

(71) Applicant: Luxembourg Institute of Health (LIH), Strassen (LU)

(72) Inventors: Xavier Dervillez, Prümzurlay (DE); Catherine Léonard, Bastogne (BE); Markus Wolfgang Ollert, Mondorf-les-Bains (LU)

(73) Assignee: Luxembourg Institute of Health (LIH), Strassen (LU)

(21) Appl. No.: 18/576,969

(22) PCT Filed: Jul. 7, 2022
(86) PCT No.: PCT/EP2022/068951

§ 371 Date: Jan. 5, 2024

(30) Foreign Application Priority Data
Jul. 7, 2021 (EP) 21184298.4

(51) Int. Cl. A61K 39/215; A61P 31/14; C07K 14/005; C12N 7/00

(52) U.S. Cl. CPC A61K 39/215; A61P 31/14; C07K 14/005; C12N 7/00; C12N 2770/00022; C12N 2770/00034

(57) ABSTRACT

The present invention provides multimeric protein complex comprising three polypeptides each comprising N- to C-terminally: (i) a receptor-binding domain (RBD) of an S1 subunit of an S protein of a coronavirus, (ii) optionally a S2 subunit of an S protein of a coronavirus; and (iii) a multimerization domain comprising a collagen-like region (CLR) of ficolin-2, wherein the multimerization domain enables the assembly of the polypeptides into a multimeric protein complex. The present invention further provides polynucleotides encoding the polypeptides of the multimeric protein complex, expression vectors, pharmaceutical compositions and uses of the multimeric protein complexes, such as a vaccine.

### TECHNICAL FIELD

The present invention is situated in the field of multimeric protein complexes and the use thereof in vaccines. More particularly, the invention relates to multimeric protein complexes comprising one or more antigen(s).

### BACKGROUND OF THE INVENTION

After more than one year of the current pandemic of coronavirus disease 2019 (COVID-19) caused by Severe Acute Respiratory Syndrome Coronavirus-2 (SARS-CoV-2), which is a threat not only to global health but also to the economies since it is dramatically affecting the socioeconomic layers of societies around the planet, multiple vaccines have been approved by the Food and Drug Administration (FDA), European Medicines Agency (EMA) and other regulatory bodies worldwide.

All the approved vaccines are either based on novel mRNA technology or on vector strategies. The mRNA vaccines, although very effective, have the disadvantage that a stringent cooling chain of −20° C. to −80° C. is necessary for vaccine delivery to the end user to prevent degradation of the vaccine—thus requiring an infrastructure that is difficult to achieve in many parts of the developing world. Vector-based vaccines have the disadvantage that every individual can only be vaccinated once with a given vaccine vector as immunity against the vector itself builds up, which can limit future vaccine efficacy when using an identical vector repeatedly.

Despite the high number of vaccine approaches currently pursued worldwide, there is still a major need for novel prophylactic or therapeutic vaccines against the SARS-CoV-2 virus.

### SUMMARY

Present inventors found that the collagen-like region (CLR) of ficolin or ficolin-like proteins, particularly the CLR of ficolin-2, is an ideal multimerization scaffold for the production of recombinant multimers of antigens of pathogens, such as the Spike protein of SARS-CoV-2 or part thereof, with native folding properties. As a result of the relatively small size of the CLR of ficolin or ficolin-like proteins, particularly of the CLR of ficolin-2, the resulting multimeric protein complex is less heavy and is closer to the native sequence of the antigen of the pathogen.

Vaccines based on the multimeric protein complexes as taught herein are efficient as they do not require a stringent cooling chain infrastructure as, for example, mRNA-based vaccines. The vaccines as taught herein are also advantageous over vector-based vaccines as the vaccine as they are protein subunit vaccines and therefore can be administered to a subject more often over a lifetime and do not interfere with other vaccinations, while vector-based vaccines can only be used for one vaccination approach per vector type.

Moreover, as the vaccines as taught herein can be administered multiple times, various arising escape variants of the virus could be addressed in a booster injection. Vaccines based on the multimeric protein complexes as taught herein are a very safe and effective vaccine technology.

The spike glycoprotein is the critical viral element that is responsible for host cell recognition, attachment, and entry for the human coronaviruses. The trimeric spikes are the transmembrane protein that undergoes dramatic structural rearrangements for binding to its host cell receptor, the angiotensin-converting enzyme 2 (ACE2), that mediate subsequent membrane fusion and virion entry. Upon binding to the ACE2 receptor, the prefusion spike trimer undergoes a dramatic conformational change where S1 domains are dissociated thereby exposing the S2 trimeric core containing the fusion machinery. S2 structural post fusion shift consists of S2 unfolding and docking of the fusion peptide to the targeted cell membrane. The transmembrane domains are bridged together, forming a long needle-like structure (e.g. as described in Ismail and Elfiky, SARS-CoV-2 spike behavior in situ: a cryo-EM images for a better understanding of the COVID-19 pandemic, Signal Transduction and Targeted Therapy, 2020). The prefusion state is generally transient and very unstable. Therefore, there is high interest in locking the S trimer into a stable prefusion conformation. Furthermore, the efficacy of current vaccines based on the highly variable pre-fusion state-stabilized spike may rapidly be altered or impaired. In contrast, S2 is more conserved among coronaviruses than S1. Antibodies induced against highly conserved stabilized-postfusion state (i) may induce broader neutralizing, longer-lasting antibody response, (ii) may reduce the likelihood of sequence altering mutations that render the immunogen ineffective, thus enhancing the cross-reactivity potential not only against SARS-CoV-2 current and future variants but also against other future coronaviruses. Ideally, a protein-based vaccine combining prefusion and post-fusion state-stabilized spikes would likely elicit broad spectrum cross-reacting neutralizing Abs.

To the inventors' surprise, different self-trimerizing collagen-like peptide scaffolds (CLRs) of ficolin or ficolin-like proteins modulate the trimeric spike of a coronavirus, preferably a SARS-CoV-2 virus, in a pre- or post-fusion conformation. More particularly, the CLR is able to influence the type of stabilized trimeric spike being formed, depending on the number of cysteines present in the scaffold. For example, a cysteine-free CLR (CLR4) scaffold allows to generate exclusively pre-fusion stabilized spikes without requiring amino acid substitutions in the S2, in contrast to current strategies using proline substitutions in S2 to get these pre-fusion stabilized spikes. More particularly, current approaches introducing up to 6 proline substitutions (F817P, A892P, A899P, A942P, K968P & V969P) generate non-natural neo-epitopes in S2 that induce specific antibodies recognizing motives that do not exist in real life. These antibodies are likely to be inefficient against authentic viruses. Using present invention, the self-trimerizing collagen-like peptide scaffold controls trimeric soluble spike stabilization either under pre- or post-fusion state without modifying its structure.

Furthermore, present invention allows the development of a vaccine that combines pre-fusion and post-fusion stabilized-trimeric spike glycoproteins. Currently, there is no vaccine under development using post-fusion stabilized-spikes. Since S2 is much less subject to mutation than pre-fusion spike among SARS-CoV-2 variants and among other coronaviruses, present invention would allow the development of a pan-coronavirus vaccine that would likely elicit antibodies that display cross-neutralizing activity against a broad range of SARS-CoV-2 variants.

To the inventors surprise, the expression of polypeptides comprising a multimerization domain consisting of the CLR of ficolin-2 and comprising only one cysteine lead to the formation of primarily pre-fusion and post-fusion stabilized-trimeric spike glycoproteins. Interestingly, present inventors found that a combination of the pre-and post-fusion form of the trimer of the Spike protein of SARS-CoV-2, preferably enriched in the post-fusion form (e.g. comprising at least 70% of the post-fusion form), (also referred to as “[S]² or “[S2]”” in the example section) as taught herein efficiently raises a concentration-dependent productive serologic responses against the SARS-CoV-2 spike antigen, preferably when used in combination with one or more adjuvants, such as a CpG adjuvant. In addition, antibodies induced by this combination of pre- and post-fusion stabilized trimeric spike glycoproteins efficiently block angiotensin-converting enzyme 2 (ACE2) binding to SARS-CoV-2 spike and the receptor-binding domain (RBD) of SARS-CoV-2 spike, reaching the range of human COVID-19 patient sera. Moreover, the combination of the pre-and post-fusion form of the trimer of the Spike protein of SARS-CoV-2 as taught herein induces efficient antibody responses in vivo and activate human complement, thus providing an additional potential for innate immune adjuvanticity through C3b/C3d deposition. In addition, the combination of the pre- and post-fusion form of the trimer of the Spike protein of SARS-CoV-2 as taught herein offers the versatility to include any new SARS-CoV-2 spike protein variant into the molecular design of a vaccine. Interestingly, the combination of the pre- and post-fusion form of the trimer of the Spike protein of SARS-CoV-2 was shown to outperform (i) the pre-fusion form of the trimer of the Spike protein of SARS-CoV-2 (also referred to as monomerc spike or “[S]¹” in the example section of present specification); as well as (ii) presumed oligomers of the trimeric post-fusion-stabilized spikes.

Accordingly, present inventors' CLR multimerization domain allows the expression of a post-fusion form of the trimer of the Spike protein of SARS-CoV-2 exposing neo-epitopes that are recognized by the anti-spike Abs from the sera. These epitopes may be hidden in the pre-fusion form of the trimer, limiting the capacity to elicit a strong neutralizing immune response. Thus, present inventors have developed a safe and highly effective 2nd generation COVID-19 protein subunit vaccine.

Furthermore, present inventors found that a total of two cysteine residues in the multimerization domain, such as by the elongation of the CLR of ficolin-2 by three amino acids comprising one cysteine, resulted in the formation of considerable fractions of presumed oligomers of the trimeric post-fusion-stabilized spikes in addition to trimeric pre-and post-fusion stabilized spikes.

Finally, present inventors found that the absence of a cysteine residue in the multimerization domain resulted in the formation of trimeric pre-fusion stabilized spikes, and a very limited number of trimeric post-fusion stabilized spikes.

Accordingly, a first aspect of the invention provides multimeric protein complexes based on polypeptides comprising a collagen-like region (CLR) of ficolin-2 and at least part of a coronavirus spike (S) protein. More particularly, the invention provides
a multimeric protein complex comprising three polypeptides, each comprising N- to C-terminally:
(i) a receptor-binding domain (RBD) of an S1 subunit of an S protein of a coronavirus,
(ii) optionally a S2 subunit of an S protein of a coronavirus; and
(iii) a multimerization domain comprising a collagen-like region (CLR) of ficolin-2, wherein the multimerization domain enables the assembly of the polypeptides into a multimeric protein complex.

In particular embodiments, the multimerization domain comprises at most 75 amino acids, preferably at most 55 amino acids, at most 48 amino acids or at most 45 amino acids.

In particular embodiments, the multimerization domain comprises from 1 to 3 cysteines, preferably one or two cysteines, more preferably one cysteine.

In particular embodiments, the multimerization domain consists of (i) the CLR of ficolin-2 (examples of such multimerization domain are referred to in the present specification as “CLR1”); or (ii) the CLR of ficolin-2 and immediately C-terminally of the CLR of ficolin-2 a peptide consisting of three amino acids of which one is a cysteine (examples of such multimerization domain are referred to in the present specification as “CLR2”), preferably wherein the peptide corresponds to the first three amino acids of the fibrinogen-like region (FLR) of ficolin-2 (e.g. amino acid sequence QPC).

In particular embodiments, the polypeptides each comprise a linker peptide C-terminally of the S2 subunit of the S protein of the coronavirus and N-terminally of the multimerization domain

In particular embodiments, at least one of the polypeptides comprises at its C-terminal end a tag, preferably wherein the tag comprises N-terminally a proteolytic cleavage site.

In particular embodiments, the polypeptides each comprise the complete S1 subunit and the S2 subunit of the S protein of the coronavirus.

In particular embodiments, the S1/S2 cleavage site is mutated, thereby preventing proteolytic processing of S protein in the S1 and S2 subunits.

A further aspect provides a polynucleotide encoding a polypeptide of the multimeric protein complex as taught herein.

In particular embodiments, the polynucleotide does not comprise a sequence encoding the signal peptide or part of the signal peptide of the coronavirus Spike protein.

A further aspect provides an expression vector comprising the polynucleotide as taught herein.

A further aspect provides a method for preparing a trimeric protein complex, comprising
(a) introducing a polynucleotide encoding a polypeptide comprising N- to C-terminally:
(i) a receptor-binding domain (RBD) of an S1 subunit of a Spike (S) protein of a coronavirus,
(ii) optionally a S2 subunit of an S protein of a coronavirus; and
(iii) a multimerization domain comprising a collagen-like region (CLR) of ficolin-2, wherein the multimerization domain enables the assembly of the polypeptides into a trimeric protein complex, into a host cell,
(b) allowing the host cell to express and secrete the polypeptides, resulting in the self-multimerization of the polypeptides into trimeric protein complexes; and
(c) separating the trimeric protein complexes from the supernatants.

A further aspect provides a trimeric protein complex obtainable by or obtained by the method for preparing a trimeric protein complex as taught herein. A further aspect provides a composition comprising a combination of protein complexes, the protein complexes comprising three polypeptides, each comprising N- to C-terminally:
(i) a RBD of an S1 subunit of an S protein of a coronavirus,
(ii) optionally a S2 subunit of an S protein of a coronavirus; and
(iii) a multimerization domain comprising a CLR of ficolin-2, wherein the polypeptides have not assembled, or the polypeptides have assembled into trimeric protein complexes by way of said multimerization domain.

A further aspect provides a pharmaceutical composition comprising the multimeric protein complex as taught herein, the polynucleotide as taught herein, the expression vector as taught herein, or the composition as taught herein, and a pharmaceutically acceptable carrier.

A further aspect provides the multimeric protein complex as taught herein, the pharmaceutical composition as taught herein, or the composition as taught herein for use as a medicament, preferably wherein the medicament is a vaccine.

A further aspect provides the multimeric protein complex as taught herein, the pharmaceutical composition as taught herein, or the composition as taught herein for use in preventing a coronavirus infection, preferably a SARS-CoV-2 infection.

These and further aspects and preferred embodiments of the invention are described in the following sections and in the appended claims. The subject-matter of the appended claims is hereby specifically incorporated in this specification.

### BRIEF DESCRIPTION OF DRAWINGS

FIG. 1 Expression vector expressing the soluble recombinant SARS-CoV-2 Spike (synthetic gene). The vector pEF-IRESpac was opened in ECORI and Not I in the multiple cloning site (MCS) to introduce the synthetic fragment composed of 1) a signal sequence, 2) the N terminal domain of the SARS-CoV-2 Spike protein, 3) the Receptor Binding Domain (RBD) of the Spike protein, 4) the Heptad Repeat 1 domain of the Spike protein, 5) the Heptad Repeat 2 domain of the Spike protein, 6) the SGGGGS (SEQ ID NO: 1) linker, 7) the Collagen-Like Region (CLR) of the human Ficolin-2 (e.g. scaffold for dimerization (CLR1) or trimerization (CLR2)), 8) the poly-histidine tag (8 successive His). The CLR scaffold region is detailed in the lower part of the drawing: the Cla I restriction site corresponding to ID aa sequence (black, underlined); the BspEI restriction site corresponding to SG aa sequence (gray, underlined); the SGGGGS (SEQ ID NO: 1) linker (gray); the 45 aa of the CLR1 (black, italic), “QPC” 3 amino acid C-terminal extension of the fibrinogen-like region including in the CLR2 (parenthesis); the SpeI restriction site corresponding to TS aa sequence (black, underlined); the BstB I overlaps with the FE aa sequence (black, underlined) and the 8 Histidine tag (black, box).

FIG. 2 Ficolin-2 collagen-like region (CLR) as dimerisation/trimerisation scaffold to produce soluble recombinant dimeric/trimeric SARS-CoV-2 Spike glycoproteins as novel protein-based vaccine candidates. A) Structure of a triple-stranded coiled-coil ficolin-2 trimeric subunit. The cysteine environment controls the triple-stranded coiled-coil formation from the G-X-Y repeats of the CLR single strands. The N-terminal “Cysteine-rich region” contains a single cysteine in position 32 (C32). The central CLR contains a single cysteine in position X of the first G-X-Y repeat (C52). The C-terminal “fibrinogen-like region” contains 6 cysteines (C98, C105, C126, C133, C257 and C270; positions of the cysteines are indicated in the precursor form of human ficolin-2 as annotated under Uniprot accession number Q15485.2). B) Cloning strategy of the SARS-CoV-2 extracellular Spike domain fused to the CLR of ficolin-2. CLR1 contains a single Cysteine C52. CLR2, in addition of cysteine C52, contains in its C-terminal end the first three amino acids from the fibrinogen-like region (QPC) that include the cysteine C98 (SEQ ID NO: 26). CLR4 corresponds to CLR1 but the single Cysteine C52 was replaced by an Alanine (C52A), and thus CLR4 contains no cysteine. C) Quatemary structure of ficolin-2. The N-terminal 25 amino acid forming the «Cysteine-rich region» of ficolin-2 (containing the cysteine C32) together with cysteine C52 of the CLR allow the covalent association of four trimeric subunits (SEQ ID NO: 38). D) Top view of the interchain disulfide bridge formation that takes place between 2 cysteines C32, 2 cysteines C52 or between 2 cysteines C32 and C52. Strand 2 is involved in the covalent association with strands 1 and 3 within each trimeric subunit, while strands 1 and 3 are involved in the covalent associations with adjacent trimeric subunits to form the dodecameric structure: the tetrameric association of trimeric subunits.

FIG. 3 Structure of Ficolin-2 and collagen-like region di-/tri-merisation scaffold CLR-variants used. Ficolin-2 signal peptide is not included in the figure. Five different scaffolds from the ficolin-2 collagen-like region were designed.
1. Scaffold 1 (CLR1) comprises the original CLR from ficolin-2 (SEQ ID NO: 25). It displays a single cysteine in position 52 (C52, nomenclatura UniProtKB Q15485.2). CLR1 generates a combination of the pre- and post-fusion form of the trimer of the Spike protein, enriched in the post fusion form.
2. Scaffold 2 (CLR2) is the scaffold 1 (CLR1) that is C-terminally extended of the first 3 amino acids (QPC) of the fibrinogen-like region that contains the Cysteine C98 (SEQ ID NO: 26). Scaffold 2 thus contains Cysteines C52 and C98.
3. Scaffold 3 (CLR3) consists of scaffold 1 (CLR) that is N-terminally extended of the 25 amino acids of the N-terminal «Cysteine-rich region» of ficolin-2 (SEQ ID NO: 27). Scaffold 3 thus displays 2 cysteines, the Cysteines C32 and C52.
4. Scaffold 4 (CLR4) is the same as scaffold 1 (CLR), with the exception of cysteine C52 was replaced by an Alanine (C52A) (SEQ ID NO: 39). Scaffold 4 does not comprise any cysteines.
5. Scaffold 5 (CLR5) is a further C-terminal extension of CLR2 of 7 amino acids beyond C98 within FLR to the C105 of FLR (SEQ ID NO: 28). CLR5 thus comprises 3 cysteines (C52, C98 and C105).

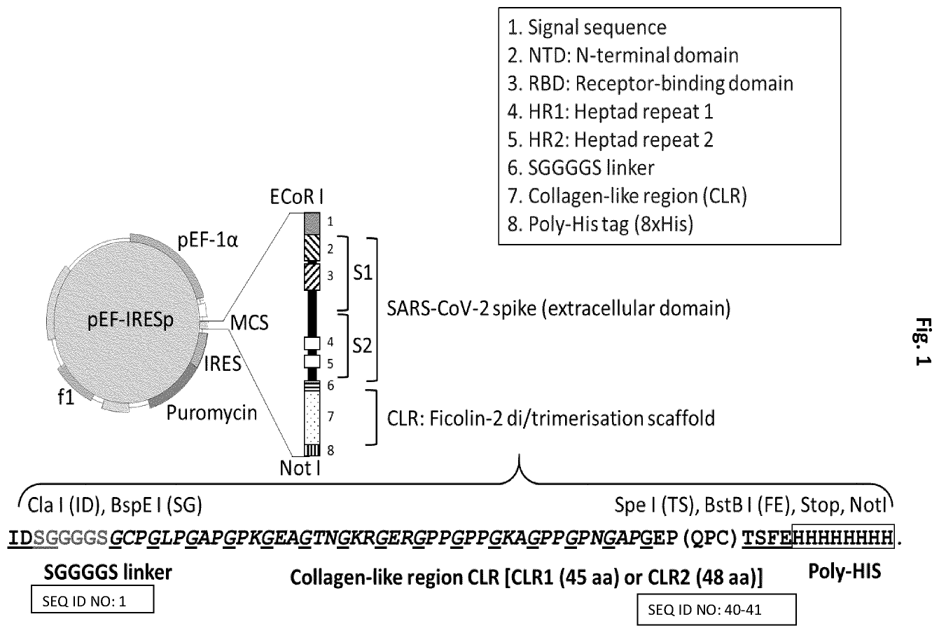

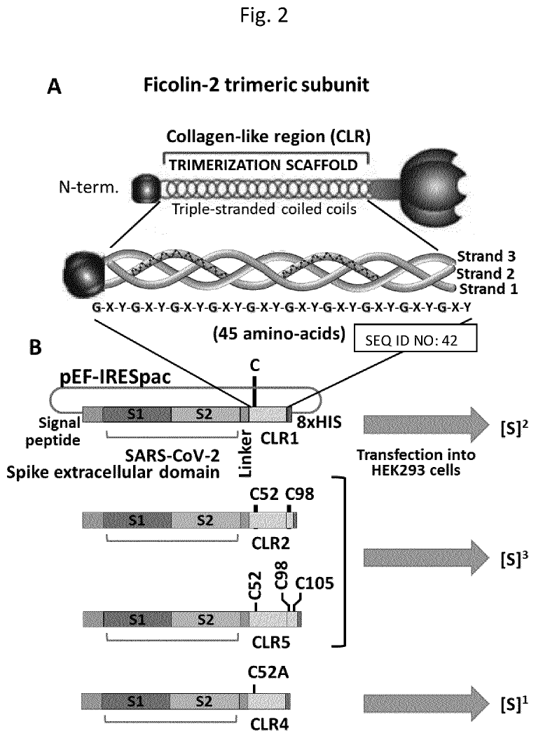

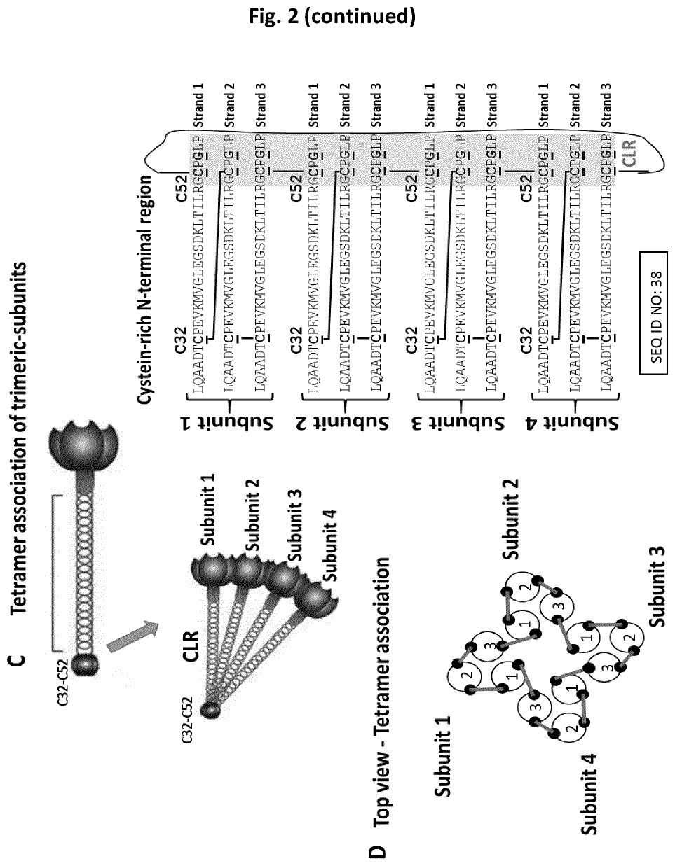

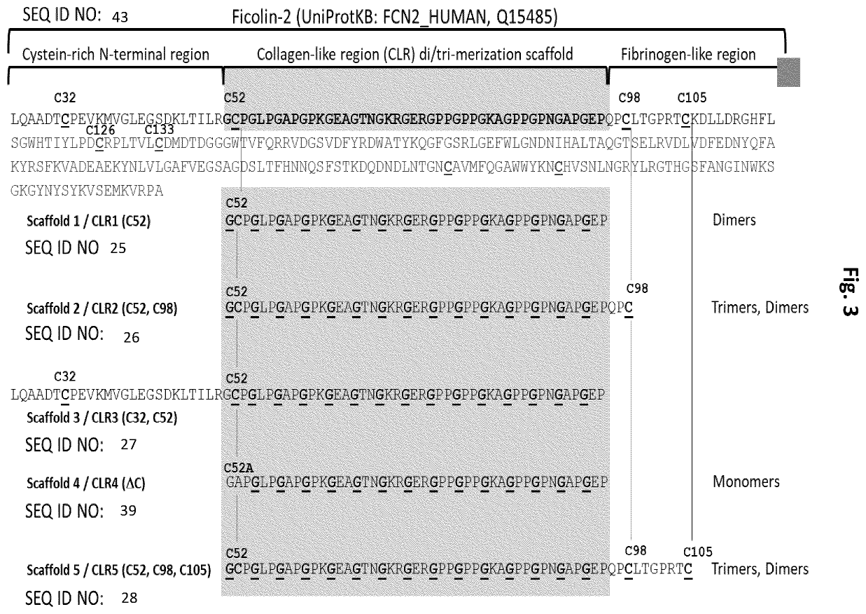

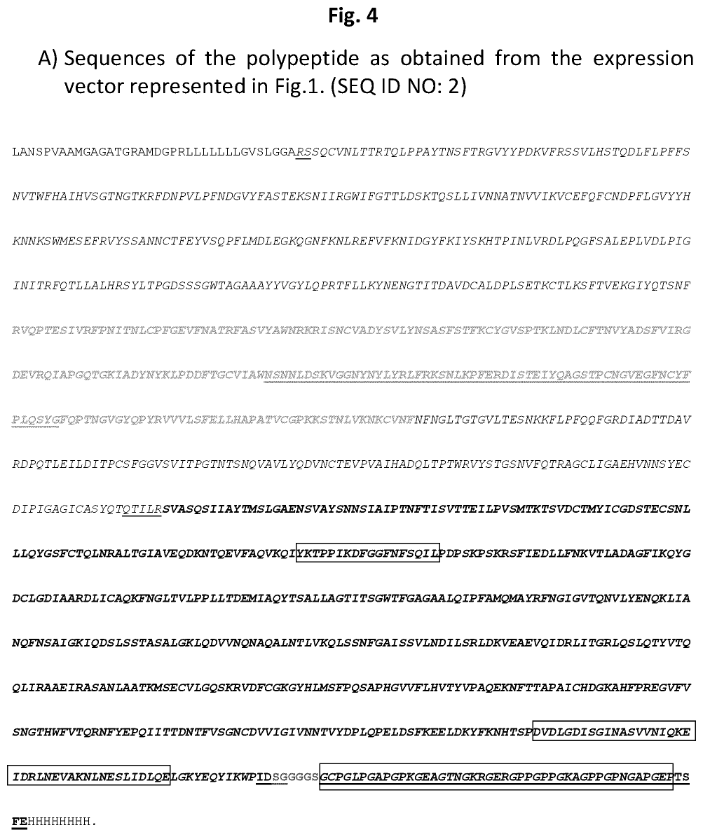

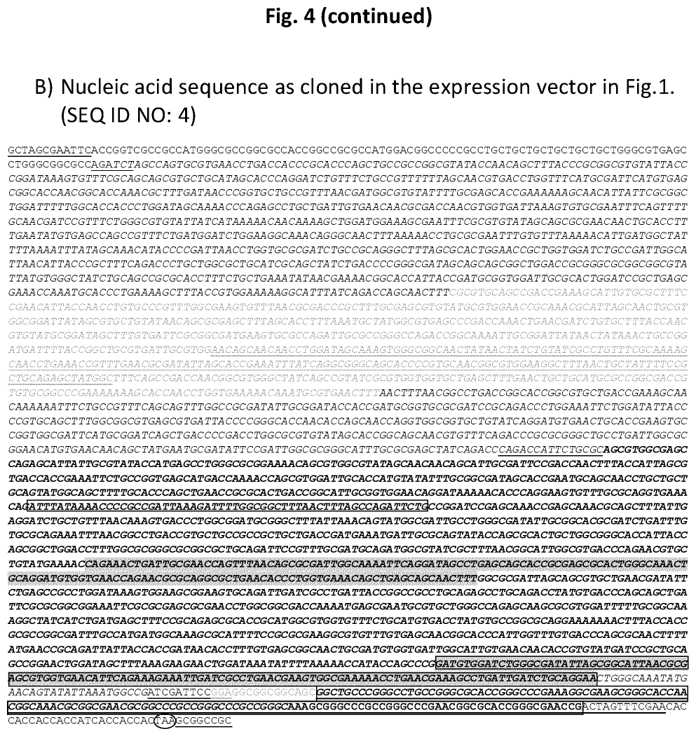

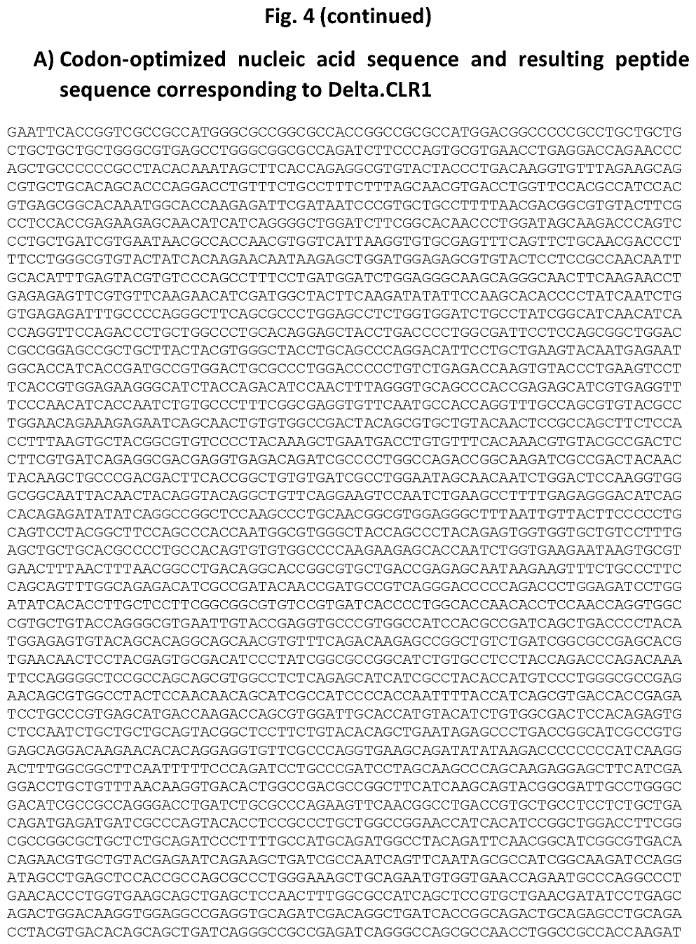

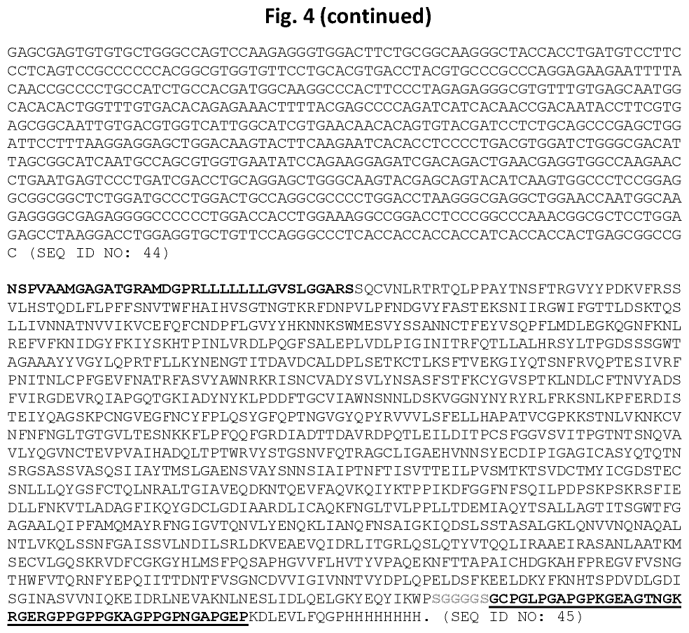

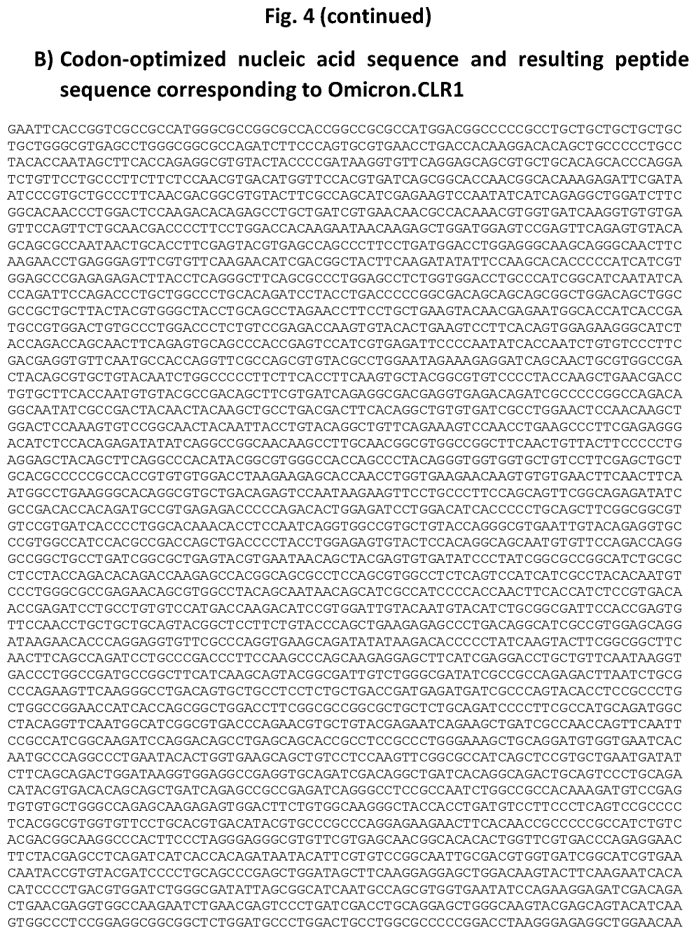

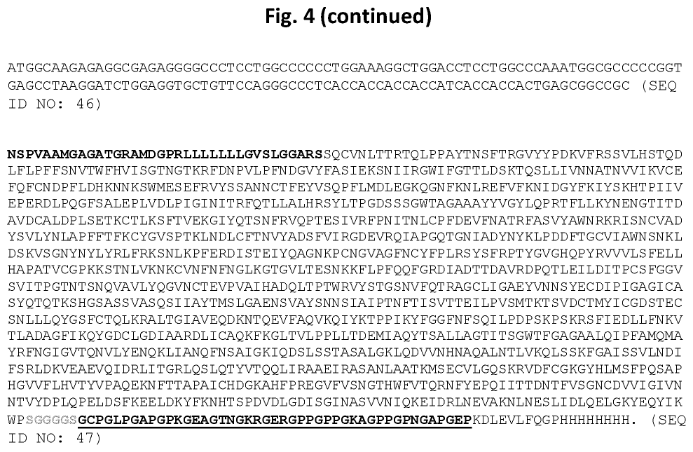

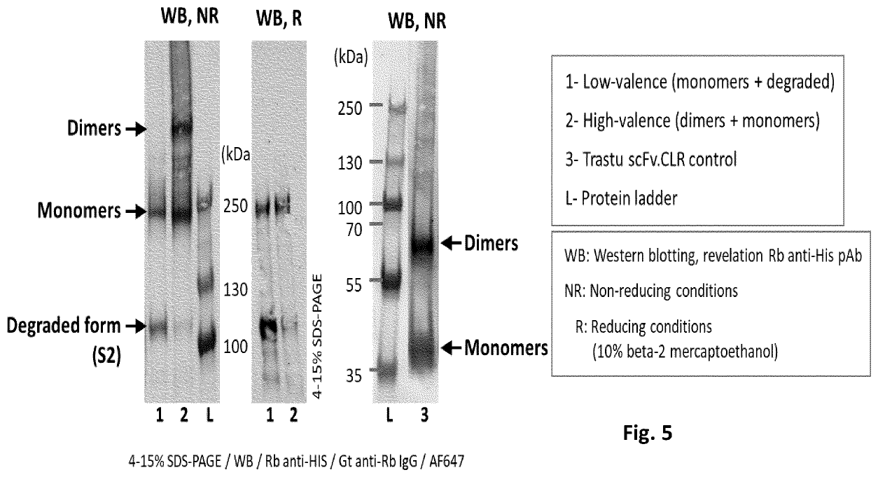

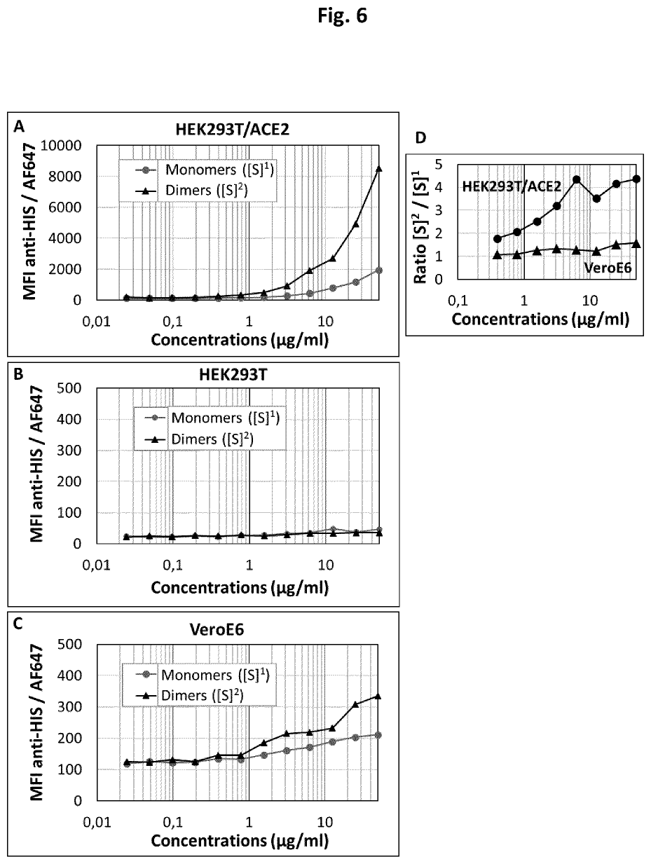

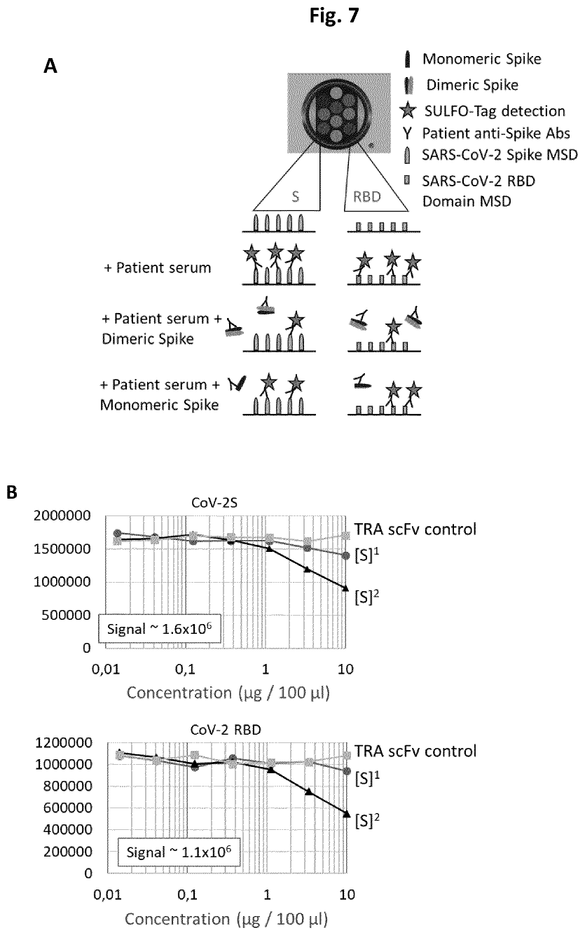

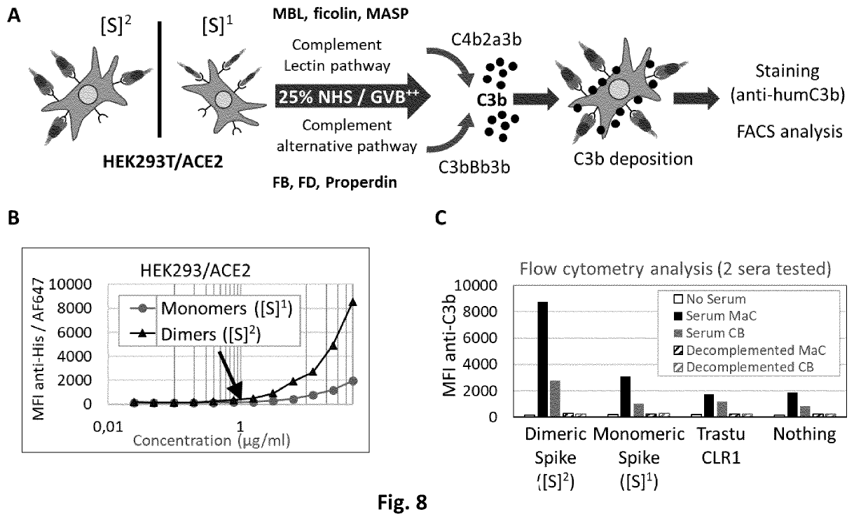

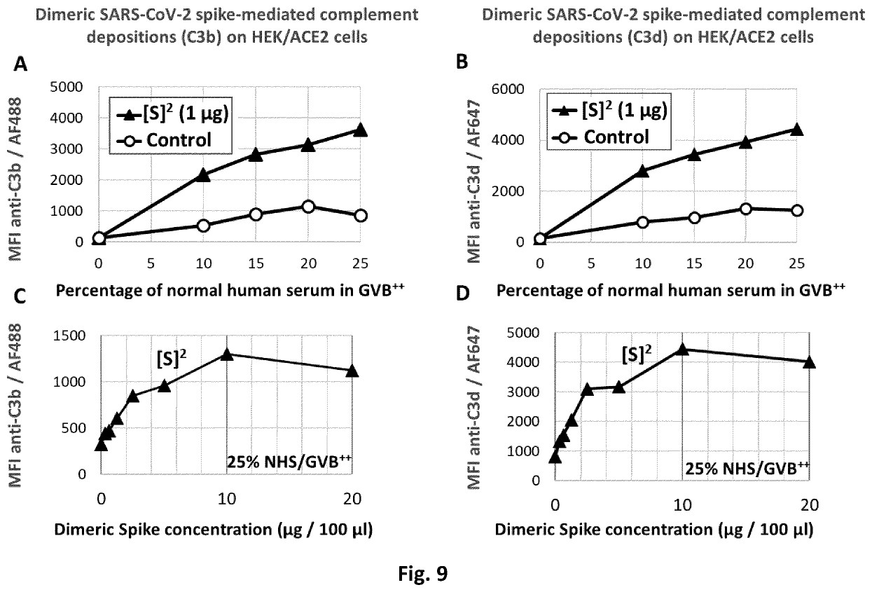

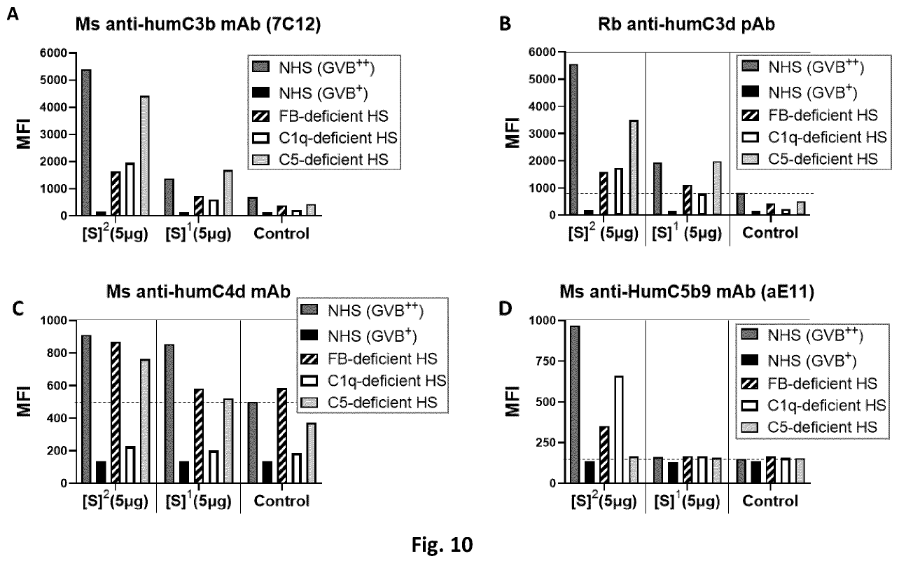

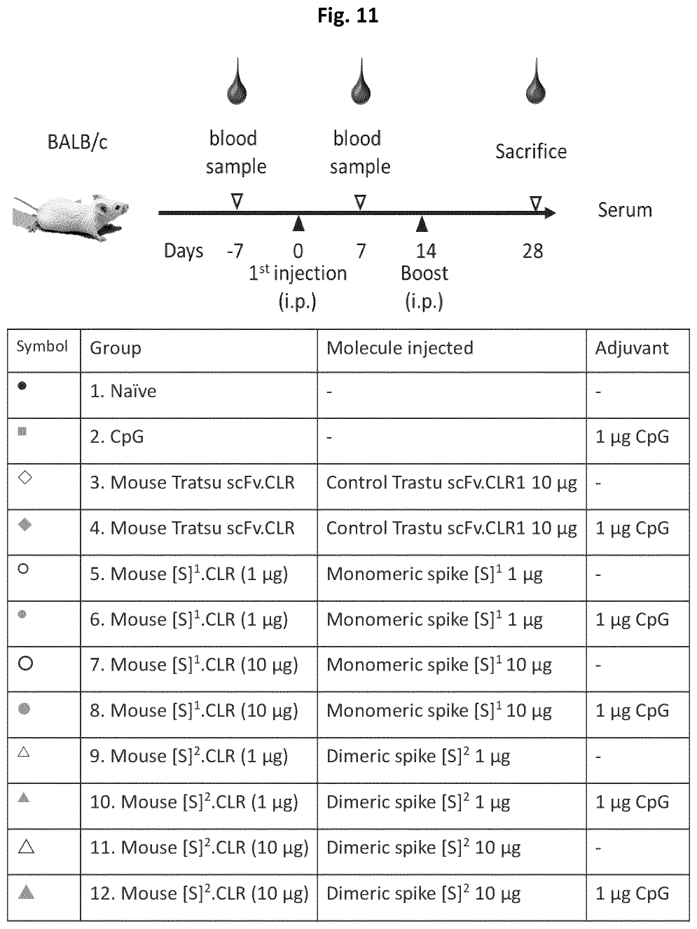

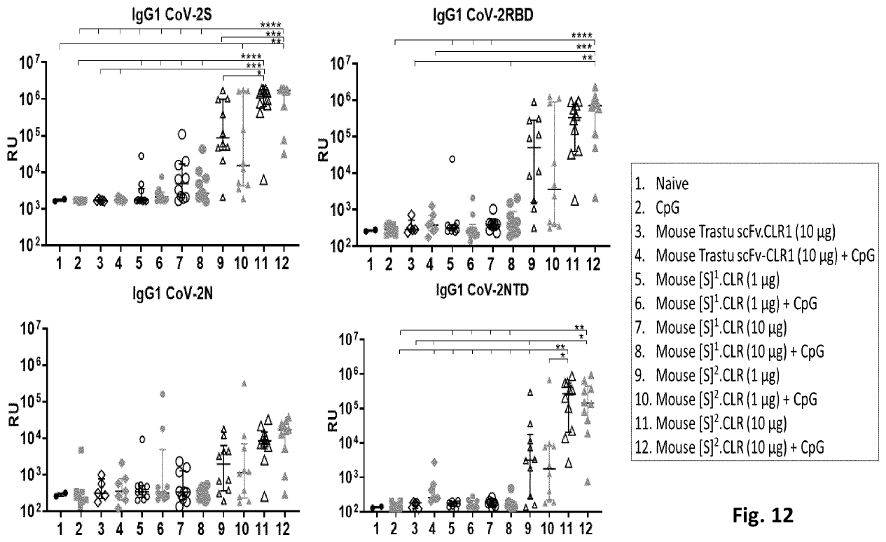

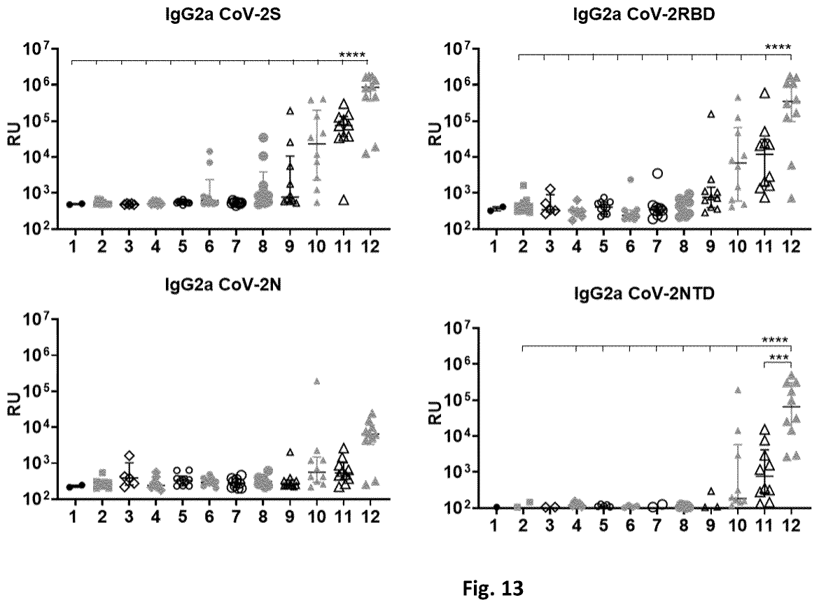

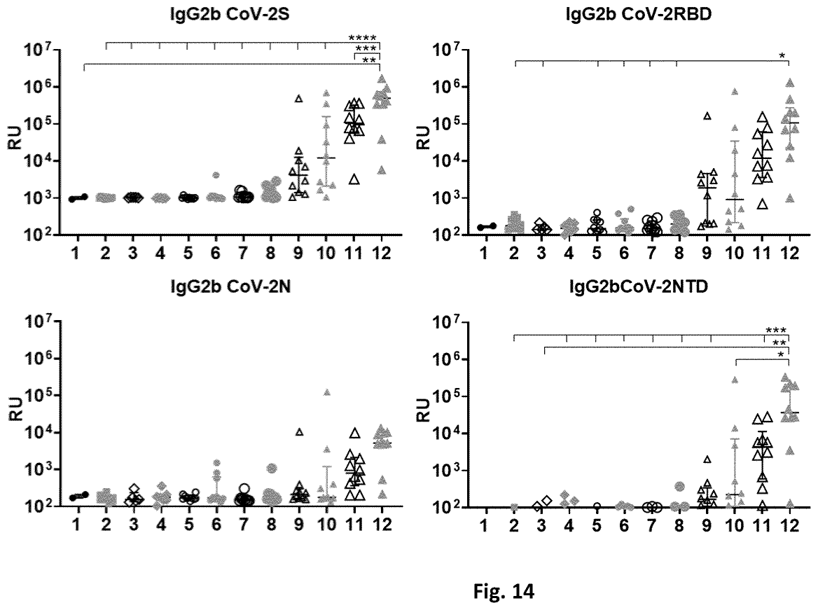

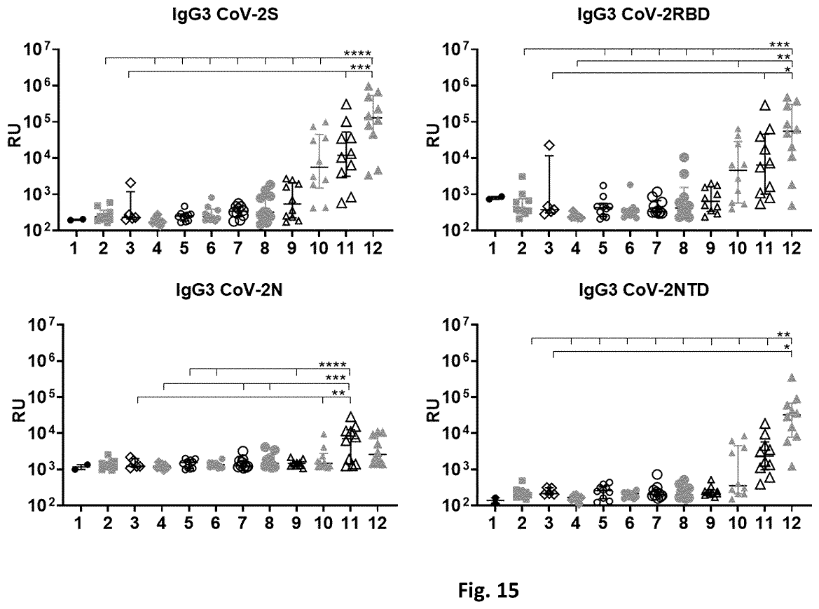

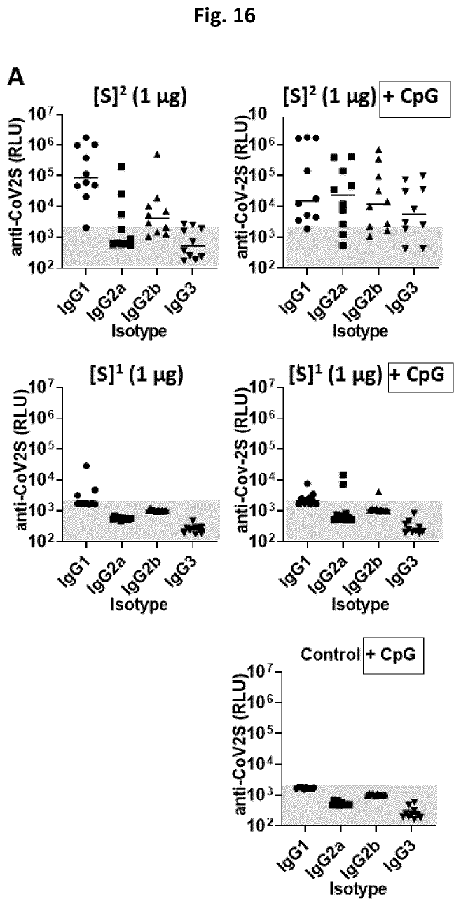

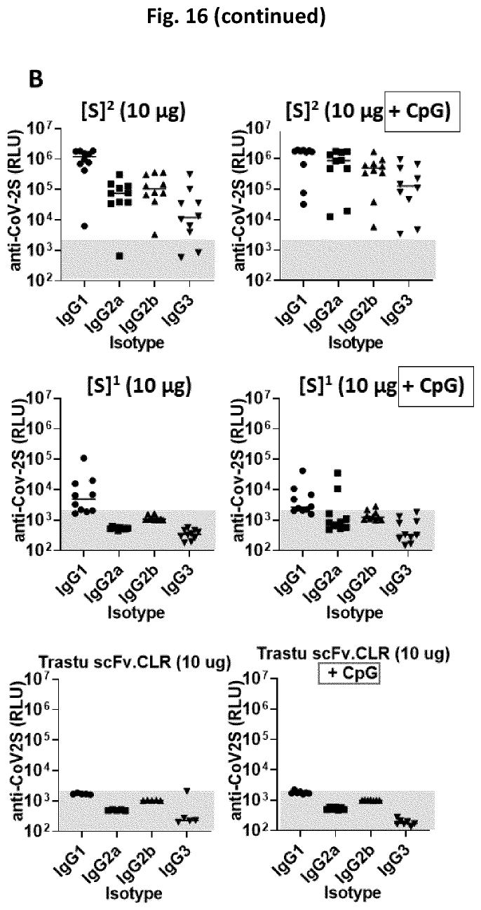

FIG. 4 A) Amino acid sequence of the Spike-CLR1 synthetic gene cloned in the pEF-IRESpac expression vector represented in FIG. 1 (SEQ ID NO: 2). Normal text layout: signal peptide between restriction site ECORI and Bgl2. Underligned RS: Bgl2 restriction site corresponding to RS aa sequence. Italic: SARS-CoV-2 Spike protein sequence with: Black: S1 domain; Grey: Receptor Binding Domain (with grey underlined: Receptor Binding Motif); Underlined QTILRS (SEQ ID NO: 6-): Mutated Furin cleavage site, modified to prevent early degradation of the protein; Bold: S2 domain; Sequence in box: Fusion peptide; Shadowed: Heptad repeat 1; Shadowed in box: Heptad repeat 2. Underligned ID: ClaI restriction site corresponding to ID aa sequence. Underligned grey SG: BspEI restriction site corresponding to SG aa sequence. Grey SGGGGS: SGGGGS (SEQ ID NO: 1) linker. Underlined Bold Italic in box: Collagen like region (CLR1, 45 aa). Underlined TS: SpeI restriction site corresponding to TS aa sequence. Underlined FE: BstBI restriction site corresponding to FE aa sequence. Final HHHHHHHH (SEQ ID NO: 3): 8×histidine tag. B) Nucleic acid sequence of the Spike-CLR1 synthetic gene cloned in the pEF-IRESpac expression vector represented in FIG. 1 (SEQ ID NO: 4). encoding the protein sequence mentioned above in FIG. 5 Part 1. Colors and symbols between the nucleic acid and related amino acid sequences were kept. Normal text layout: signal peptide between restriction sites NheI G/CTAGC, Eco RI G/AATTC and Bgl2 A/GATCT. Underligned A/GATCT: Bgl2 restriction site at the end of the signal peptide. Italic: SARS-CoV-2 Spike protein sequence with: Black: S1 domain; Grey: Receptor Binding Domain (with grey underlined: Receptor Binding Motif); Underlined CAGACCATTCTGCGC (SEQ ID NO: 5; S1/S2 mutated cleavage site of Spike protein of SARS-CoV-2): Mutated QTILRS (SEQ ID NO: 6) Furin cleavage site; Bold: S2 domain; Sequence in box: Fusion peptide; Shadowed: Heptad repeat 1; Shadowed in box: Heptad repeat 2. Underligned AT/CGAT: ClaI restriction site. Underligned grey T/CCGGA: BspEI restriction site. Grey TCCGGAGGCGGCGGCAGC (SEQ ID NO: 7; linker): SGGGGS (SEQ ID NO: 1) linker. Underlined Bold Italic in box: Collagen like region (CLR1). Underlined A/CTAGT: SpeI site. Underligned restriction TT/CGAA: BstBI restriction site. CACCACCACCACCATCACCACCAC (SEQ ID NO: 8): Poly histidine tag. TAA in oval: Stop codon. Final GC/GGCCGC: NotI restriction site. C) Nucleic acid sequence and resulting peptide sequence corresponding to the spike of Delta variant with CLR1 scaffold. The construction followed the same description as above. The nucleic acid sequence starts with the Eco RI G/AATTC restriction site and ends with the final GC/GGCCGC: NotI restriction site. The peptide sequence starts with the signal peptide sequence (bold), the normal text englobes Delta spike S1, S2 and RBD domains and the sequence ends with the SGGGGS linker (grey) and the CLR1 peptide (bold underlined). Final HHHHHHHH (SEQ ID NO: 3): 8×histidine tag. D) Nucleic acid sequence and resulting peptide sequence corresponding to the spike of Omicron variant with CLR1 scaffold. The construction followed the same description as above. The nucleic acid sequence starts with the Eco RI G/AATTC restriction site and ends with the final GC/GGCCGC: NotI restriction site. The peptide sequence starts with the signal peptide sequence (bold), the normal text englobes Omicron spike S1, S2 and RBD domains and the sequence ends with the SGGGGS linker (grey) and the CLR1 peptide (bold underlined). Final HHHHHHHH (SEQ ID NO: 3): 8×histidine tag.

FIG. 5 SARS-CoV-2 spike purification using immobilised metal chelate chromatography (His-Trap purification): His-Trap purified final batches of dimeric ([S]²), monomeric ([S]¹) and control Trastuzumab scFv.CLR1 used for the vaccination of mice. Final purified batches used for the vaccination in BALB/c mice were called “low-valence” and “high-valence” batches, consisting respectively of (i) [S]¹ monomers+degraded forms (S2 domain (i.e. S2 subunit of the Spike protein)) and (ii) [S]² dimers+[S]¹ monomers. Low-valence and high-valence batches were analysed using SDS-PAGE, Western blotting (WB) under non-reducing (NR) or reducing (R) conditions, revealed with a rabbit anti-His pAb and a goat anti-rabbit IgG AF647-conjugated.

FIG. 6. Dose-response binding of soluble recombinant dimeric ([S]²) versus monomeric ([S]¹) SARS-CoV-2 spike glycoproteins and to HEK293T/ACE2⁺/TMPRSS2⁺ or VeroE6 cells. HEK293T/ACE2⁺/TMPRSS2⁺/mCherry cells (“HEK293T/ACE2”) (A), VeroE6 kidney cells from C. aethiops (C) and control HEK293T (B) were incubated with 2-fold serial dilutions of [S]² or [S]¹ and then stained with an anti-His and analysed using flow cytometry.

FIG. 7. Comparative study of the ability of soluble recombinant SARS-CoV-2 dimeric ([S]²) versus monomeric ([S]¹) spike glycoproteins to compete with the binding of anti-SARS-CoV-2 Spike antibodies from COVID-19 patient serum to coated SARS-CoV-2 Spike (S) or SARS-CoV-2-S-RBD using MSD multiplex assay. The ability of [S]² versus [S]¹ (and control TRA scFv.CLR1) to prevent the interaction of anti-spike antibodies from pooled CoviD-positive patient sera with SARS-CoV-2 Spike (S) or SARS-CoV-2-S-RBD was investigated using MSD multiplex assay. A) Schematic summary of the experimental setting. B) Results of the competitive binding between [S]² (but not [S]¹) and SARS-CoV-2 spike and RBD by anti-SARS-CoV-2 spike antibodies from CoviD patients.

FIG. 8. Activation of human complement by recombinant soluble SARS-CoV-2 spike [S]¹ and [S]². A) Experimental setting of complement activation cascade leading to the cleavage of C3 by C3-convertases of the lectin (C4b2a) and/or alternative (C3bBb) complement pathways. Complement activation was detected using flow cytometry with a labelled anti-human C3/C3b/iC3b murine monoclonal antibody. B) The arrow indicates the concentration of [S]² and [S]¹ used to perform the complement activation assay (10 μg/ml). C) Mean Fluorescence Intensity (MFI) signal for C3b corresponding to C3b depositions on HEK293/ACE2 cells upon incubation with normal human serum (NHS).

FIG. 9 Flow cytometry analysis of the dimeric spike-mediated C3b depositions on HEK293T/ACE2 cells. A-B) HEK293T/ACE2 cells were incubated with (i) 10 μg/ml purified [S]², followed by (ii) 0, 10, 15, 20 and 25% NHS. C-D) HEK293T/ACE2 cells were incubated with (i) 2-fold serial dilutions of purified [S]² (starting at 200 μg/ml), followed by (ii) 25% NHS. Cells were then stained with either (i) a mouse anti-human C3/C3b/iC3b mAb (A, C) or (ii) a rabbit anti-human C3d pAb (B, D).

FIG. 10 Comparative study of [S]² versus [S]¹-mediated complement activation on HEK293T/ACE2 cells using several serum conditions. Five μg of purified [S]², [S]¹ or no molecule as control were incubated with HEK293T/ACE2 cells (150,000 cell/well) for 30 min. at 4° C. Cells were then incubated for 30 min. at 37° C. with 25% (i) NHS in gelatin veronal buffer supplemented with Ca⁺⁺ and Mg⁺⁺ (GVB⁺⁺), (ii) NHS in GVB⁺ [contains EGTA 5 mM and MgCl₂ 3 mM: the classical/lectin complement pathways are inactivated], (iii) commercial Factor B-depleted human serum. Cells were then stained with (A) a mouse anti-human C3/C3b/iC3b mAb, or (B) a rabbit anti-human C3d pAb, or (C) a mouse anti-human C4d mAb or (D) a mouse anti-human C5b9 mAb. Cells were then stained with a secondary goat anti-mouse pAb AF488-conjugated or anti-rabbit pAb AF647-conjugated. Cells were then analysed using flow cytometry.

FIG. 11 Vaccination of BALB/c mice. Mice were injected twice intraperitoneally with 200 μl of different vaccination solutions as depicted in the table of the lower part of FIG. 12. The 12 groups received injections of the following solution: 1. No injection (naïve mice); 2. 1 μg CpG; 3. 10 μg Trastu scFv.CLR, without any adjuvant; 4. 10 μg Trastu scFv.CLR with 1 μg CpG; 5. 1 μg of the monomeric form of the recombinant protein [S]¹-CLR1 (“[S]¹”), without any adjuvant; 6. 1 μg [S]¹ with 1 μg CpG; 7. 10 μg [S]¹ without any adjuvant; 8. 10 μg [S]¹ with 1 μg CpG; 9. 1 μg of the dimeric form of the recombinant protein [S]²-CLR1 (“[S]²”), without any adjuvant; 10. 1 μg [S]² with 1 μg CpG; 11. 10 μg [S]² without any adjuvant; 12. 10 μg [S]² with 1 μg CpG. The indicated symbols for the groups are used in all subsequent graphs.

FIG. 12 IgG1 serology in vaccinated mice. Specific IgG1 antibodies against SARS-CoV-2 Spike (CoV-2S), S-RBD domain (CoV-2RBD), SARS-CoV-2 Nucleocapsid (CoV-2N) and S N-terminal domain (CoV-2NTD) were measured in the serum of the 12 groups of vaccinated mice (see figure legend of FIG. 11 for description of the groups 1-12) via the MSD multiplex approach. RU: Relative units. One way ANOVA statistical analysis: **p<0.01; ***p<0.001; ****p<0.0001.

FIG. 13 IgG2a serology in vaccinated mice. Specific IgG2a antibodies against SARS-CoV-2 Spike (CoV-2S), S-RBD domain (CoV-2RBD), SARS-CoV-2 Nucleocapsid (CoV-2N) and S N-terminal domain (CoV-2NTD) were measured in the serum of the 12 groups of vaccinated mice (see figure legend of FIG. 11 for description of the groups 1-12) via the MSD multiplex approach. RU: Relative units. One way ANOVA statistical analysis: ****p<0.0001, ***p<0.001.

FIG. 14 IgG2b serology in vaccinated mice. Specific IgG2b antibodies against SARS-CoV-2 Spike (CoV-2S), S-RBD domain (CoV-2RBD), SARS-CoV-2 Nucleocapsid (CoV-2N) and S N-terminal domain (CoV-2NTD) were measured in the serum of the 12 groups of vaccinated mice (see figure legend of FIG. 11 for description of the groups 1-12) via the MSD multiplex approach. RU: Relative units. One way ANOVA statistical analysis: *p<0.05; **p<0.01; ***p<0.001; ****p<0.0001.

FIG. 15 IgG3 serology in vaccinated mice. Specific IgG3 antibodies against SARS-CoV-2 Spike (CoV-2S), S-RBD domain (CoV-2RBD), SARS-CoV-2 Nucleocapsid (CoV-2N) and S N-terminal domain (CoV-2NTD) were measured in the serum of the 12 groups of vaccinated mice (see figure legend of FIG. 11 for description of the groups 1-12) via the MSD multiplex approach. RU: Relative units. One way ANOVA statistical analysis: *p<0.05; **p<0.01; ***p<0.001; ****p<0.0001.

FIG. 16 Summary of serology responses in mice vaccinated with SARS-CoV-2 spike antigen. The profiles of anti-SARS-CoV-2 Spike (S)-specific IgG1, IgG2a, IgG2b and IgG3 of the mice, which received different vaccination solutions (see figure legend of FIG. 11 for the description of the groups), obtained using the MSD multiplex approach, are presented in one graph per type of vaccination, for 1 μg (A) or 10 μg (B) injected monomer or dimer. The structure of the molecules used for the vaccination (recombinant dimeric spike [S]², or recombinant monomeric spike [S]¹ or control scaffold molecule Trastu scFv) are represented on the left of the corresponding graphs. RLU: Read Log Update.

FIG. 17 Summary of serology responses in mice vaccinated with SARS-CoV-2 spike antigen. The profiles of anti-SARS-CoV-2 Spike receptor binding domain (S-RBD)-specific IgG1, IgG2a, IgG2b and IgG3 of the mice, which received different vaccination solutions (see figure legend of FIG. 11 for the description of the groups), obtained using the MSD multiplex approach, are presented in one graph per type of vaccination, for 1 μg (A) or 10 μg (B) injected monomer or dimer. The structure of the molecules used for the vaccination (recombinant dimeric spike [S]², or recombinant monomeric spike [S]¹ or control scaffold molecule Trastu scFv) are represented on the left of the corresponding graphs.

FIG. 18 Surrogate Neutralization Assay. The ability of antibodies from vaccinated mice to inhibit the binding of sulfo-tagged ACE2 to SARS-CoV-2 Spike (S) (CoV-2 S) and SARS-CoV-2 S-RBD (CoV-2 RBD) was tested using the MSD technique, in a surrogate neutralization assay. Serum was isolated from mice which received: 1. 1 μg CpG; 2. 10 μg Trastu scFv.CLR1, without adjuvant; 3. 10 μg Trastu scFv.CLR1 with 1 μg CpG; 4. 1 μg [S]¹, without adjuvant; 5. 1 μg [S]¹ with 1 μg CpG; 6. 10 μg [S]¹ without adjuvant; 7. 10 μg [S]¹ with 1 μg CpG; 8. 1 μg of [S]² without adjuvant; 9. 1 μg [S]² with 1 μg CpG; 10. 10 μg [S]² without adjuvant; 11. 10 μg [S]² with 1 μg CpG (for coherence, the symbols in front of the groups are the same as in FIG. 11); The results include for comparison the inhibition induced by serum from convalescent COVID-19 patients who developed a strong form of the disease (12) and with the inhibition induced by serum from convalescent COVID-19 patients who developed a mild form of the disease (13). The graphs show the inhibition obtained when sera were diluted 1/5 (A), 1/25 (B) and 1/50 (C). Results depict competition with ACE2 for binding to SARS-CoV-2S (CoV-2 S) protein (left graphs of A-C) or for SARS-CoV-2S RBD (CoV-2 RBD) domain (right graphs of A-C).

FIG. 19 Correlation of serologic antibody levels and surrogate binding inhibition of huACE2 to SARS-CoV-2 S and S-RBD induced by vaccination with Spike [S]¹ and [S]². Using the MSD technology results as in FIG. 19, the ability of the anti-Spike and anti-RBD specific IgG1 (A) and IgG2a (B) antibodies to compete with ACE2 was plotted in function of their serology titres in vaccinated mice.

FIG. 20 Flow cytometry analysis of the binding of 4 soluble recombinant trimeric SARS-CoV-2 Spike glycoproteins: Supernatants from transient transfections were tested on HEK-293T/ACE2, stained with an anti-His or an anti-SARS-CoV-2 Spike and analysed using flow cytometry. Three spikes [“All spike mutations from B1.1.7 (UK), B1.351 (South-African) and P.1 (Brazilian) variants”, B1.351 Spike (South-African) and the 5 mutation Spike (L18F, K417N, E484K, N501Y and D614G)] fused to CLR2 were compared to the original Wuhan spike fused to CLR1.

FIG. 21 Serological detection of IgG1, IgG2a, IgG2b and IgG3 against SARS-CoV-2 by MSD assay. 1. No injection (naïve mice, n=5); 2. 500 μg Alum+2 μg CpG (n=5); 3. 10 μg Dimeric Beta spike alone (n=5); 4. 10 μg Dimeric Beta spike+CpG (n=10); 5. 10 μg Dimeric Beta spike+Alum (n=10); 6. 10 μg Dimeric Beta spike+Alum and CpG (n=10); 7. 10 μg Trimeric Beta spike+CpG (n=10); 8. 10 μg Trimeric Beta spike+Alum (n=10); 9. 10 μg Trimeric Beta spike+Alum and CpG (n=10).

FIG. 22. Analysis of the neutralizing capacities of anti-spike Abs. A-D: MSD serology and SARS-CoV-2 surrogate virus neutralization assay: Evaluation of the concentration (μg/ml) of neutralizing anti-spike SARS-CoV-2 S or RBD antibodies in sera from mice vaccinated with either the dimeric or the trimeric Beta spike glycoproteins (A-B) or their neutralizing capacity (%) (C-D). Nr. 1 to 9 (see figure legend of FIG. 21), 10. Patient CHL COVID 19+(n=4), 11. Patient COVID19 severe (n=3), and 12. Pool patient COVID19-. E-F: Neutralization of syncytium formation between (E) WT spike- or (F) Beta spike-expressing HEK293T & VeroE6 cells by elicited anti-spike antibodies in sera from vaccinated mice with either soluble (1) dimeric (“Dimer mice group”) or (2) trimeric (“Trimer mice group”) Beta spikes [with (Alum+CpG) adjuvantization]. As controls, (3) a WHO standard neutralizing serum was used as well as (4) a highly neutralizing serum from a SARS-CoV-2 positive convalescent donor whose neutralizing antibody titres were ranked in the 10 highest among a cohort of 80 Covid-positive convalescent donors CoV-Pos (Top 10/80). For each mouse serum, half-maximum inhibitory serum dilution (IC₅₀) was determined. Two-tailed unpaired t tests was used to compared dimer & trimer groups with p<0.05. G) Neutralization of infection of VeroE6 cells against authentic SARS-CoV-2 Beta variants antibodies from mice sera that were vaccinated with either soluble (1) dimeric or (2) trimeric Beta spikes by [with Alum+CpG adjuvants]. On hundred TCDI₅₀ of authentic SARS-CoV-2 Beta virus were used. As controls, present inventors used (3) a convalescent serum from an individual recently infected with the delta variant, (4) the CoV-Pos 10/80 serum as for A & B, and (5) the serum from a vaccinated donor collected 15 days after the second dose of the Pfizer vaccine (Pfz #2, D15). Due to the heterogeneity of the neutralizing antibody response in the trimer group, no significant statistical difference was found between dimer and trimer groups. H-J: Neutralization of syncytium formation between (H) WT spike-, (I) Beta spike- and (J) Delta spike-expressing HEK293T & VeroE6 target cells by competition with 6 different purified soluble recombinant spikes: (1) monomeric Beta spike, (2) dimeric Beta spike, (3) trimeric Beta spike, (4) dimeric Beta spike with the alternative C4bp C-terminal β-chain dimerization scaffold, (5) dimeric Delta spike and (6) dimeric BA.1 Omicron spike. Half-maximum inhibitory soluble spike concentrations (IC₅₀) were determined from the dose-response non-linear [log (agonist) vs. response] (four parameters) best-fit curves in 3 independent experiments. One-way ANOVA with Tukey's multiple comparisons test was performed to evaluate the significant statistical difference between groups. This assay correlates with the relative affinity of the 6 spikes for VeroE6 cell surface ACE2 receptor, and their efficacy to compete with spike-expressing HEK293T cells to prevent syncytium formation.

FIG. 23 (A-B) Comparative binding efficacy of anti-spike Abs from sera from 4 different convalescent Covid-positive donors to 5 purified soluble recombinant spikes coated to ELISA plates: (1) dimeric Beta spike, (2) trimeric Beta spike, (3) dimeric Beta spike with the alternative C4bp C-terminal β-chain dimerization scaffold, (4) dimeric Delta spike and (5) dimeric BA.1 Omicron spike, A) Series of 12 serial two-fold dilutions of the 4 sera starting at serum dilution 1/50 were performed to determine the serum titration to get 50% of the maximum signal (EC₅₀). The first 1:50 dilution is always saturating and was thus used as reference for 100% initial binding to calculate the % initial signal for the other dilutions. The ELISA was revealed using a goat anti-human IgG horseradish peroxidase-conjugated pAb. The present figure depicts the example of serum titration from #6 donor among serum titrations of 4 convalescent donors and of a control SARS-CoV2-negative donor (data not shown). B) Given the huge difference in the anti-spike titers between the 4 convalescent donors, we expressed EC₅₀ ratios, by dividing the EC₅₀ for a given spike by the EC₅₀ for the Beta.CLR1 dimers. Ratios superior to 1 mean that the given spike has a higher binding capacity of anti-spike antibodies compared to the Beta.CLR1 dimeric spikes, and vice versa. In graph B, one-way ANOVA non-parametric tests with multiple comparisons were used to determine significant statistical differences. In this ELISA, the degree of binding efficacy of elicited anti-spike antibodies from convalescent sera to a given spike molecule correlates with the accessibility of the spike epitopes to these anti-spike antibodies that recognize either linear or cryptic epitopes in the spike. A particular spike folding displaying highest binding capacity could be considered as optimized vaccine spike candidate, with the exception of the monomeric spike, which displays the highest spatial binding surface but is non-immunogenic. This has something to do with the binding capacity for cryptic anti-spike antibodies that are typically those with highest neutralizing capacities.

FIG. 24 Protective efficacy evaluation of the soluble Beta.CLR1 spike glycoprotein protein subunit vaccine candidate in K18-hACE2 transgenic mice in a vaccination/challenge preclinical trial with authentic SARS-CoV-2 Beta variant. Mice (n=10) were vaccinated at D0 and D14 with 10 μg soluble Beta.CLR1 spike with [Alum+CpG] adjuvantization. Control mice (n=10) received the adjuvants alone. At D28, the non-vaccinated and vaccinated groups were infected with SARS-CoV-2 Beta virus (10² CPU). A third control group (n=3) received the adjuvants alone but were not infected. A) From D28 to D42 (end of the experiment), the individual body weight was followed up daily for the 3 groups. A one-way Anova with Tukey's multiple comparisons statistical analysis was performed to show the statistically significant difference of body weight changes between the 3 groups, and in particular between non-vaccinated/infected & vaccinated/infected groups. B) Survival curve for the 3 groups of mice. The grey area represent the post inoculation time. Because of the small sample, alternative, two-sided Fischer's exact test was preferred as statistical significance test using the contingency table in the figure to calculate the significance of the deviation from a null hypothesis [P value was 0.00309 (**)].

FIG. 25 Purification of soluble recombinant SARS-CoV-2 spike glycoproteins. For the production of monomeric, dimeric and trimeric spikes, a one-step elution after His-Trap capture was combined to a step of concentration and size-exclusion chromatography with a S200 sephadex column. A) The monomeric Beta spike was obtained by expression of the protein with the CLR4 scaffold. One example of SDS-PAGE and silver-staining is given, with elution fractions 11 to 23 corresponding to the 2 peaks represented on the chromatogram. B) The dimeric Beta spike was obtained by expression of the protein with the CLR1 scaffold (example of purification on the left SDS-PAGE) or alternatively with the C4bpβ scaffold (SDS-PAGE with silver staining and corresponding peaks in the chromatogram on the right). C) The trimeric spike was obtained by expression of the protein with the CLR2 scaffold (example of purification on the left SDS-PAGE) or alternatively with the CLR5 scaffold (SDS-PAGE with silver staining and corresponding peaks in the chromatogram on the right).

FIG. 26 Cryo-EM imaging of the “dimeric beta” sample. A-B) Cryo-EM analysis & 2D classification of the “dimeric beta” sample showed without a doubt two populations, a minor population corresponding to the trimeric pre-fusion-stabilized spike glycoproteins (with 1 or 2 “RBD-up”) (A), and a main population corresponding to the trimeric post-fusion-stabilized spike glycoproteins (B), respectively. C) A low-resolution 3D reconstruction of the main population of the “dimeric beta” spike was further performed, clearly showing a stabilized post-fusion trimeric spike. This 3D reconstruction is moreover consistent with the post-fusion SARS-CoV-2 trimeric spike cryo-EM resolution found in the literature, such as described in Fan et al., 2020, Nature communications. Cryo-EM analysis of the post-fusion structure of the SARS-CoV spike glycoprotein. Five-glycosylation sites can be clearly seen on asparagines N1098, N1134, N1158, N1173 and N1194, followed by the C-terminal CLR1 trimerization scaffold. D) left: SEC-elution fractions 11-23 of the “monomeric beta” sample (with CLR4 scaffold lacking cysteine); middle: SEC-elution fractions 13-19 of the “dimeric beta” sample (with CLR1 displaying a single cysteine C52); right: SEC-elution fractions 6-10 of the “trimeric beta” sample (with CLR5 displaying 3 cysteines C52, C98 & C105).

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Present inventors have demonstrated that the collagen-like region (CLR) of ficolin or ficolin-like proteins, particularly the CLR of ficolin-2, and its flanking regions, are an ideal multimerization scaffold for the production of recombinant multimers of antigens of pathogens, such as the Spike protein of SARS-CoV-2 or part thereof, with native folding properties of the antigens, to elicit an appropriate immunological reaction against these antigens in a subject.

Vaccines based on the multimeric protein complexes. preferably a combination of pre-fusion and post-fusion stabilized trimeric spike glycoproteins enriched in the post-fusion stabilized spikes, as taught herein are a very safe and effective vaccine technology, being able to induce efficient antibody responses in vivo as well as providing an additional potential for innate immune adjuvanticity through C3b/C3d deposition. Furthermore, such vaccines as taught herein are efficient as they do not require a stringent cooling chain infrastructure as, for example, mRNA-based vaccines, and can be injected multiple times over a life-time with different antigens, and therefore are advantageous over vector-based vaccines.

Accordingly, a first aspect provides a fusion polypeptide comprising
(i) a multimerization domain comprising or consisting essentially of a collagen-like region (CLR) of ficolin or a ficolin-like protein;
(ii) at least one antigen of a pathogen; wherein the antigen is located N-terminally and/or C-terminally, preferably C-terminally, of the at least one antigen of the pathogen.

If the fusion polypeptide comprises more than one antigen of a pathogen, the fusion polypeptide may comprise a first antigen N-terminally of the multimerization domain and a second antigen C-terminally of the multimerization domain, wherein the first and second antigens may be identical or different.

The fusion polypeptide as taught herein may multimerize into multimer, such as a dimer, or trimer. The fusion polypeptide as taught herein is also referred to in the present specification as a monomer or monomeric protein complex, when occurring in a non-multimerized form.

The fusion polypeptide as taught herein may be used to generate multimers with a high valence of an antigen. For example, if the fusion polypeptide comprises an identical antigen N-terminally and C-terminally of the multimerization domain, dimers of such polypeptide may have a valence of 4 of such antigen and trimers a valence of 6.

A further aspect provides a multimeric protein complex comprising at least two, preferably two or three polypeptides, even more preferably three polypeptides (i.e. a trimeric protein complex), each comprising N- to C-terminally
(i) an antigen of a pathogen; and
(ii) a multimerization domain comprising a collagen-like region (CLR) of ficolin or a ficolin-like protein.

The multimerization domain enables the assembly of the polypeptides into a multimeric protein complex, such as a trimeric protein complex. Accordingly, in particular embodiments, in the multimeric protein complex, the polypeptides are assembled into trimeric protein complexes through the multimerization domain. For example, in the trimeric protein complex, three of the polypeptides, respectively, are assembled through the multimerization domain.

The term “protein” as used throughout this specification generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native proteins, such as, e.g., amino acid deletions, additions and/or substitutions. The term contemplates both full-length proteins and protein parts or fragments, e.g., naturally-occurring protein parts that ensue from processing of such full-length proteins.

The term “polypeptide” as used throughout this specification generally encompasses polymeric chains of amino acid residues linked by peptide bonds. Hence, especially when a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

The term “peptide” as used throughout this specification preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

A peptide, polypeptide or protein can be naturally occurring, e.g., present in or isolated from nature, e.g., produced or expressed natively or endogenously by a cell or tissue and optionally isolated therefrom. A peptide, polypeptide or protein can be recombinant, i.e., produced by recombinant DNA technology, and/or can be, partly or entirely, chemically or biochemically synthesised. Without limitation, a peptide, polypeptide or protein can be produced recombinantly by a suitable host or host cell expression system and optionally isolated therefrom (e.g., a suitable bacterial, yeast, fungal, plant or animal host or host cell expression system), or produced recombinantly by cell-free translation or cell-free transcription and translation, or non-biological peptide, polypeptide or protein synthesis.

In particular embodiments, the multimeric protein complex as taught herein is a recombinant multimeric protein complex (i.e. not naturally occurring in nature).

Due to intrinsic instability typical of class I fusion proteins, the Sars-Cov-2 spike protein tends to prematurely refold to the post-fusion conformation, reducing the number of trimers generated and compromising immunogenic properties. The present inventors have found that the multimeric protein complexes of the invention are particularly suitable for the presentation of the SARS-CoV-2 spike antigen which naturally occurs as a trimer. Indeed, the multimeric presentation appears to be capable of mimicking the natural conformation. The present observations with the SARS-CoV-2 spike antigen, demonstrate that this technology is suitable for antigens that occur in nature as a multimer, preferably as a trimer. Non-limiting examples of such antigens include spike proteins of different viruses (e.g. the Spike protein of a Severe Acute Respiratory Syndrome coronavirus, the E2 protein of the SARS corona virus, the glycoprotein B (gB) of herpes simplex virus type 1 (HSV-1), the vesicular stomatitis virus (VSV) G-protein, an Epstein-Barr-Virus gB protein, the Baculovirus gp64, gp 120/gp41 from human immunodeficiency virus 1 (HIV-1), gp41 protein from Simian immunodeficiency virus 1 (SIV-1), a protein from the Murine leukemia virus (MLV), a protein from the Feline Leukemia virus (FeLV), an Ebola virus protein, a Marburg virus protein, a protein from the Middle East Respiratory Syndrome (MERS) virus, a protein from the murine hepatitis virus (MHV-2) a protein of the Dengue virus, a protein of the Sindbis virus, a protein of a Lassa virus, or a protein (e.g. hemagglutinin) of an influenza virus.

Protective immunity against SARS-CoV-2 and other coronaviruses is believed to depend on neutralizing antibodies (NAbs) that target the viral spike (S) protein. In particular, NAbs specific for the N-terminal S1 domain—which contains the angiotensin-converting enzyme 2 (ACE2) receptor-binding domain—have previously been shown to prevent viral infection in several animal models.

In particular embodiments, the antigen of the pathogen comprises, consists essentially of or consists of the Spike protein of a coronavirus or part thereof.

In particular embodiment, the antigen of the pathogen comprises, consists essentially of or consists of a receptor-binding domain (RBD) of an S1 subunit of a Spike (S) protein of a coronavirus. The RBD of an S1 subunit of a Spike protein of a coronavirus may comprise, consist essentially of or consist of amino acids 319-541 of SEQ ID NO: 9 (spike protein of SARS-CoV-2). The receptor-binding motive of the RBD may comprise, consist essentially of or consist of amino acids 3437-508 of SEQ ID NO: 9.

In particular embodiment, the polypeptides of the multimeric protein complex do not comprise the S2 subunit of the S protein of the coronavirus.

In particular embodiment, the antigen of the pathogen comprises, consists essentially of or consists of N- to C-terminally
(i.i) a receptor-binding domain (RBD) of a S1 subunit of a Spike (S) protein of a coronavirus, and
(i.ii) a S2 subunit of a S protein of a coronavirus.

In particular embodiment, the antigen of the pathogen comprises, consists essentially of or consists of the complete S1 subunit of a Spike (S) protein of a coronavirus and the complete S2 subunit of a S protein of a coronavirus.

In particular embodiments, the coronavirus is COVID-19 (or SARS-CoV-2).

The Spike protein may be the Spike protein of any variant of the SARS-CoV-2 virus. For example, the Spike protein is the Spike protein from the SARS-CoV-2 isolate Wuhan-Hu-1 as as annotated under NCBI Genbank accession number MN908947.3, the Spike protein from the Alpha variant (also known as the UK variant) of the SARS-CoV-2 virus (e.g. VOC 202012/01, B.1.1.7) , the Spike protein from the Gamma variant (also known as the Brazilian-Japanese variant) of the SARS-CoV-2 virus (e.g. B.1.1.28 or P1), the Spike protein of the Beta variant (also known as the the South African variant) of the SARS-CoV-2 virus (e.g. VOC 501Y.V2, B. 1.351), the Spike protein of the Epsilon variant (also known as the Californian variant of the SARS-CoV-2 virus (e.g. B. 1.427 or B.1.429), the Spike protein of the Iota variant (also known as the New York variant) of the SARS-CoV-2 virus (e.g. B.1.526 or B.1.526.1), the Spike protein of the Eta variant (also known as the UK/Nigeria variant) of the SARS-CoV-2 virus (e.g. B.1.525), the Spike protein of the Kappa variant (also known as the Indian variant) of the SARS-CoV-2 virus (e.g. B.1.617, B.1.617.1, B.1.617.2 or B.1617.3), the Spike protein of the Zeta variant (also known as the Brazilian variant) of the SARS-CoV-2 virus (e.g. P.2), the Theta variant of the SARS-CoV-2 virus (e.g. P3), the Lambda variant of the SARS-CoV-2 virus (e.g. C. 37), the Mu variant of the SARS-CoV-2 virus (e.g. B.1.621), the Delta variant of the SARS-CoV-2 virus (e.g. B.1.617.2), or the Omicron variant of the SARS-CoV-2 virus (e.g. B.1.1.529).

In preferred embodiments, the SARS-CoV-2 Spike protein is the Spike protein of the Beta, Delta or Omicron variant of SARS-CoV-2.

In particular embodiments, the multimeric protein complex may comprise two or three different polypeptides, wherein each polypeptide comprises a RBD of an S1 subunit of a Spike protein of a different SARS-CoV-2 virus. For example, the multimeric protein complex may comprise one polypeptide comprising a RBD of an S1 subunit of a Spike protein of the Beta variant of SARS-CoV-2 and one polypeptide comprising a RBD of an S1 subunit of a Spike protein of the Delta variant of SARS-CoV-2.

An exemplary amino acid sequence of SARS-CoV-2 Spike protein is annotated under Uniprot (www.uniprot.org) accession number P0DTC2.1 and is depicted below:

(SEQ ID NO: 9)
MFVFLVLLPLVSsqcvnlttrtqlppaytnsftrgvyypdkvfrssvlhstqdlflpffsnvtwfhaihvsgtngtkrfdnpvlpfndgvyfa
steksniirgwifgttldsktqsllivnnatnvvikvcefqfcndpflgvyyhknnkswmesefrvyssannctfeyvsqpflmdlegkqgnfknlre
fvfknidgyfkiyskhtpinlvrdlpqgfsaleplvdlpiginitrfqtllalhrsyltpgdsssgwtagaaayyvgylqprtfllkynengtitdavdc
aldplsetkctlksftvekgiyqtsnfrvqptesivrfpnitnlcpfgevfnatrfasvyawnrkrisncvadysvlynsasfstfkcygvsptklndlc
ftnvyadsfvirgdevrqiapgqtgkiadynyklpddftgcviawnsnnldskvggnynylyrlfrksnlkpferdisteiyqagstpcngvegfncyfp
lqsygfqptngvgyqpyrvvvlsfellhapatvcgpkkstnlvknkcvnfnfngltgtgvltesnkkflpfqqfgrdiadttdavrdpqtleilditpcs
fggvsvitpgtntsnqvavlyqdvnctevpvaihadqltptwrvystgsnvfqtragcligaehvnnsyecdipigagicasyqtqtnsprrarSVASQ
SIIAYTMSLGAENSVAYSNNSIAIPTNFTISVTTEILPVSMTKTSVDCTMYICGDSTECSNLLLQYGSFCTQL
NRALTGIAVEQDKNTQEVFAQVKQIYKTPPIKDFGGFNFSQILPDPSKPSKRSFIEDLLFNKVTLADAG
FIKQYGDCLGDIAARDLICAQKFNGLTVLPPLLTDEMIAQYTSALLAGTITSGWTFGAGAALQIPFAMQ
MAYRFNGIGVTQNVLYENQKLIANQFNSAIGKIQDSLSSTASALGKLQDVVNQNAQALNTLVKQLSSNF
GAISSVLNDILSRLDKVEAEVQIDRLITGRLQSLQTYVTQQLIRAAEIRASANLAATKMSECVLGQSKRVD
FCGKGYHLMSFPQSAPHGVVFLHVTYVPAQEKNFTTAPAICHDGKAHFPREGVFVSNGTHWFVTQRN
FYEPQIITTDNTFVSGNCDVVIGIVNNTVYDPLQPELDSFKEELDKYFKNHTSPDVDLGDISGINASVVNI
QKEIDRLNEVAKNLNESLIDLQELGKYEQYIKWPWYIWLGFIAGLIAIVMVTIMLCCMTSCCSCLKGCCS
CGSCCKFDEDDSEPVLKGVKLHYT.

The signal peptide is indicated in bold. The S1/2 cleavage site is underlined. The S1 subunit is indicated in lowercase and the S2 subunit is indicated in italics.

The genetic variations in the genetic variants of SARS-CoV-2 can be assessed on the Centers for Disease Control and Prevention website https://www.cdc.gov/coronavirus/2019-ncov/variants/variant-info.html. For example, the Spike protein of the Californian variant B.1.427 of the SARS-CoV-2 virus typically comprises the following modifications: L452R and D614G. The Spike protein of the Californian variant B.1.427 of the SARS-CoV-2 virus typically comprises the following modifications: S13I, W152C, L452R and D614G. The Spike protein from the B.1.351 variant typically comprises the following modifications: D80A, D215G, 241del, 242del, 243del, K417N, E484K, N501Y, D614G, A701V. The Spike protein from the P1 variant typically comprises the following modifications: L18F, T20N, P26S, D138Y, R190s, K417T, E484K, N501Y, D614G, H655Y, T1027I. The Spike protein from the B.1.1.7 variant typically comprises the following modifications: 69del, 70del, 144del (E484K*), (S494P*), N501Y, A570D, D614G, P681H, T716I, S982A, D1118H, (K119N*). *indicates the mutations which are detected in some sequences but not all. The person skilled in the art will understand that, for example, 70del refers to the deletion of the amino acid at position 70 of the Spike protein, and that, for example, N501Y designates a replacement of the N residue at position 501 of the Spike protein by a Y residue. The mutations are mutations compared to the sequence of the Spike protein of the ancestor SARS-CoV-2 isolate Wuhan-Hu-1. In particular embodiments, the S1 subunit of a Spike (S) protein of the coronavirus comprises, consists essentially of or consists of the amino acid sequence having at least 90%, at least 95%, such as at least 96%, at least 97%, at least 98%, or at least 99%, preferably 100%, sequence identity with the sequence as defined by SEQ ID NO: 10.

A person skilled in the art is well aware of methods and tools to verify sequence homology, sequence similarity or sequence identity between different sequences of amino acids or nucleic acids. Non-limiting examples of such methods and tools are Protein BLAST (https://blast.ncbi.nlm.nih.gov/Blast.cgi), ClustalW2 (https://www.ebi.ac.uk/Tools/msa/clustalw2/), SIM alignment tool (https://web.expasy.org/sim/), TranslatorX (http://translatorx.co.uk/) and T-COFFEE (https://www.ebi.ac.uk/Tools/msa/tcoffee/). The percentage of identity between two sequences may show minor differences depending on the algorithm choice and parameters.

The term “sequence identity” as used herein refers to the relationship between sequences at the nucleotide or amino acid level. The expression “ % identical” is determined by comparing optimally aligned sequences. e.g. two or more, over a comparison window wherein the portion of the sequence in the comparison window may comprise insertions or deletions as compared to the reference sequence for optimal alignment of the sequences. The reference sequence does not comprise insertions or deletions. A reference window is chosen and the “% identity” is then calculated by determining the number of nucleotides (or amino acids) that are identical between the sequences in the window, dividing the number of identical nucleotides (or amino acids) by the number of nucleotides (or amino acids) in the window and multiplying by 100. Unless indicated otherwise, the sequence identity is calculated over the whole length of the reference sequence. A skilled person is aware of the related, yet different interpretations in the art of the terms “similarity”, “homology”, and “identity” (explain in detail in e.g. Pearson, Current protocols in bioinformatics, 2014).

In particular embodiments, the S2 subunit of a Spike (S) protein of the coronavirus comprises, consists essentially of or consists of the amino acid sequence having at least 90%, at least 95%, such as at least 96%, at least 97%, at least 98%, or at least 99%, preferably 100%, sequence identity with the sequence as defined by SEQ ID NO: 11.

As described elsewhere in the present specification, in the multimeric protein complex of the present invention the signal peptide of the Spike protein of the coronavirus, or part thereof may be deleted completed, and the S1/S2 cleavage site may be mutated.

Accordingly, in particular embodiments, the polypeptide of the multimeric protein complex as taught herein comprises an amino acid sequence having at least 90%, at least 95%, such as at least 96%, at least 97%, at least 98%, or at least 99%, preferably 100%, sequence identity with the sequence as defined by SEQ ID NO: 9;
(i) wherein the first 11 amino acid residues of SEQ ID NO: 9 are deleted; and
(ii) optionally wherein the S1/S2 cleavage site is mutated, preferably wherein the RRAR (SEQ ID NO: 12) sequence at position 682-685 of SEQ ID NO: 9 is amended to the sequence GSAS (SEQ ID NO: 13) or the QTNSPRRRAR (SEQ ID NO: 14) sequence at position 677-685 of SEQ ID NO: 9 is replaced by the sequence QTILR (SEQ ID NO: 15).

In other words, in particular embodiments, the polypeptide of the multimeric protein complex as taught herein comprises an amino acid sequence having at least 90%, at least 95%, such as at least 96%, at least 97%, at least 98%, or at least 99%, preferably 100%, sequence identity with the sequence as defined by SEQ ID NO: 16.

To cover one or more variants of the Spike protein of the SARS-CoV-2 coronavirus, one or more deletions or substitutions can be introduced in the sequence to design multimeric protein complexes for use in a global vaccine against SARS-CoV-2.

For example, the Spike protein may comprise deletions/mutations such as occurring in the P1, B 1.351 and/or B1.1.7 Spike protein variants.

In particular embodiments, the polypeptide of the multimeric protein complex as taught herein comprises an amino acid sequence as defined by SEQ ID NO: 9.
(i) wherein the L residue at position 18 of SEQ ID NO: 9 is substituted by a F residue, the T residue at position 20 of SEQ ID NO: 9 is substituted by a N residue, the P residue at position 26 of SEQ ID NO: 9 is substituted by a S residue, the Hand V residues at positions 69 and 70 of SEQ ID NO: 9 are deleted, the D residue at position 80 of SEQ ID NO: 9 is substituted by a A residue, the D residue at position 138 of SEQ ID NO: 9 is substituted by a Y residue, the Y residue at position 144 of SEQ ID NO: 9 is deleted, the R residue at position 190 of SEQ ID NO: 9 is substituted by S residue, the LLA sequence at position 242-244 of SEQ ID NO: 9 are deleted, the R residue at position 246 of SEQ ID NO: 9 is substituted by I residue, the K residue at position 417 of SEQ ID NO: 9 is substituted by an N or T residue, the E residue at position 484 at position 484 of SEQ ID NO: 9 is substituted by a K residue, the N residue at position 501 of SEQ ID NO: 9 is substituted by a Y residue, the A residue at position 570 of SEQ ID NO: 9 is substituted by a D residue, the D residue at position 614 of SEQ ID NO: 9 is substituted by a G residue, the H residue at position 655 of SEQ ID NO: 9 is substituted by a Y residue, the P residue at position 681 of SEQ ID NO: 9 is substituted by a H residue, the A residue at position 701 of SEQ ID NO: 9 is substituted by a V residue, the T residue at position 761 of SEQ ID NO: 9 is substituted by a I residue, the S residue at position 982 of SEQ ID NO: 9 is substituted by an A residue, the T residue at position 1027 of SEQ ID NO: 9 is substituted by a I residue and/or the D residue at position 1118 of SEQ ID NO: 9 is substituted by a H residue;
(ii) preferably wherein the first 11 amino acid residues of SEQ ID NO: 9 are deleted; and
(iii) optionally wherein the S1/S2 cleavage site is mutated, preferably wherein the RRAR (SEQ ID NO: 12) sequence at position 682-685 of SEQ ID NO: 9 is amended to the sequence GSAS (SEQ ID NO: 13) or the QTNSPRRRAR (SEQ ID NO: 14) sequence at position 677-685 of SEQ ID NO: 9 is replaced by the sequence QTILR (SEQ ID NO: 15).

In particular embodiments, the polypeptide of the multimeric protein complex as taught herein comprises an amino acid sequence as defined by SEQ ID NO: 9,
(i) wherein the L residue at position 18 of SEQ ID NO: 9 is substituted by a F residue, the D residue at position 80 of SEQ ID NO: 9 is substituted by a A residue, the LLA sequence at position 242-244 of SEQ ID NO: 9 are deleted, the R residue at position 246 of SEQ ID NO: 9 is substituted by I residue, the K residue at position 417 of SEQ ID NO: 9 is substituted by an N residue, the E residue at position 484 at position 484 of SEQ ID NO: 9 is substituted by a K residue, the N residue at position 501 of SEQ ID NO: 9 is substituted by a Y residue, the D residue at position 614 of SEQ ID NO: 9 is substituted by a G residue, and the A residue at position 701 of SEQ ID NO: 9 is substituted by a V residue;
(ii) preferably wherein the first 11 amino acid residues of SEQ ID NO: 9 are deleted; and
(iii) optionally wherein the S1/S2 cleavage site is mutated, preferably wherein the RRAR (SEQ ID NO: 12) sequence at position 682-685 of SEQ ID NO: 9 is amended to the sequence GSAS (SEQ ID NO: 13) or the QTNSPRRRAR (SEQ ID NO: 14) sequence at position 677-685 of SEQ ID NO: 9 is replaced by the sequence QTILR (SEQ ID NO: 15).

In particular embodiments, the Spike protein comprises, consists essentially of, or consists of an amino acid sequence as defined by SEQ ID NO: 9.
(i) wherein the L residue at position 18 of SEQ ID NO: 9 is substituted by a F residue, the K residue at position 417 of SEQ ID NO: 9 is substituted by an N or T residue, the E residue at position 484 at position 484 of SEQ ID NO: 9 is substituted by a K residue, the N residue at position 501 of SEQ ID NO: 9 is substituted by a Y residue and the D residue at position 614 of SEQ ID NO: 9 is substituted by a G residue;
(ii) preferably wherein the first 11 amino acid residues of SEQ ID NO: 9 are deleted; and
(iii) optionally wherein the RRAR (SEQ ID NO: 12) sequence at position 682-685 of SEQ ID NO: 9 is amended to the sequence GSAS (SEQ ID NO: 13).

In particular embodiments, the Spike protein may be encoded by the nucleic acid sequence as defined by SEQ ID NO: 17.

It is common in the art that for the production of soluble recombinant trimeric Spikes 4 to 6 mutations are introduced in the S2 region of the Spike protein of the SARS-CoV-2 coronavirus, namely mutations F817P, A892P, A942P, K986P, V987P and A899P, in order to “freeze” the Spike in a prefusion stabilized state.

The multimerization scaffold as taught herein is capable of forming a soluble recombinant native-like multimeric Spike of a coronavirus, in order to mimic as much as possible the original structure of the Spike. Such multimers are able to elicit neutralizing antibodies with a high neutralization potential and recognizing preferentially conformational epitopes in the native trimeric Spike. The neutralizing antibodies (nAbs) against COVID 19 are classified in 4 groups according to the domain recognized in the trimeric spike. There are nAbs (i) that recognize the N-terminal domain (NTD) of S1, (ii) that bind to the RBD site distal to the ACE2 receptor binding site, (iii) that compete with the receptor binding motif (RBM) and (iv) that bind to S2 domain, including to newly exposed epitopes revealed by postfusion structural rearrangements. Their neutralizing activities can take place at different time points of the fusion process: prevention of binding of the trimeric Spike to ACE2, or the ACE2-dependent Spike structural rearrangements following its binding to ACE2, or its ectodomain (HR1/HR2) activation leading to membrane fusion.

In particular embodiments, the Spike protein comprised in the polypeptide of the multimeric protein complex as taught herein does not comprise mutations F817P, A892P, A942P, K986P, V987P and A899P. By allowing the recombinant spikes to take all the native conformational states, there is higher chance to have anti-Spike antibodies produced that recognize a broader spectrum of spike conformation states, leading to higher neutralizing activities. Accordingly, in particular embodiments, the Spike protein comprised in the polypeptide of the multimeric protein complex as taught herein comprises a F residue at position 817 of SEQ ID NO: 9, an A residue at position 892 of SEQ ID NO: 9, an A residue at position 942 of SEQ ID NO: 9, a K residue at position 986 of SEQ ID NO: 9, a V residue at position 987 of SEQ ID NO: 9 and/or an A residue at position 899 of SEQ ID NO: 9.

In other words, in particular embodiments, the polypeptide of the multimeric protein complex as taught herein comprises an amino acid sequence having at least 90%, at least 95%, such as at least 96%, at least 97%, at least 98%, or at least 99%, preferably 100%, sequence identity with the sequence as defined by SEQ ID NO: 18-20 (SEQ ID NO: 18: Codon-optimised “all mutations from the UK, South-African & Brazilian spike variants”; SEQ ID NO: 19: Codon-optimised B.1.351 South-African Spike variant: SEQ ID NO: 20: Codon-optimised L18.K417.E484.N501.D614 Five mutation Spike).

In particular embodiments, if both the complete S1 subunit and the complete S2 subunit of the coronavirus are present, the S1/S2 cleavage site is mutated, thereby preventing proteolytic processing of S protein in the S1 and S2 subunits.

In particular embodiments, such as wherein the polynucleotide as taught herein comprises the complete S1 subunit and the complete S2 subunit of the S protein of the coronavirus, the S1/2 cleavage site is mutated from the amino acid sequence RRAR (SEQ ID NO: 12) to the amino acid sequence SGAG (SEQ ID NO: 21), such as described in McCallum et al., Structure-guided covalent stabilization of coronavirus spike glycoprotein trimers in the closed formation, Nature structural and molecular biology, 2020, to GSAS (SEQ ID NO: 13), or to a single R, such as described in Xiong et al., A thermostable, closed SARS-CoV-2 spike protein trimer, Natural Structural & Molecular Biology, 2020 or Walls A. C., Structure, Function, and antigeneicity of the SARS-CoV-2 Spike Glycoprotein, Cell, vol. 181(2), 2020.

In particular embodiments, such as wherein the polynucleotide as taught herein comprises the complete S1 subunit and the complete S2 subunit of the S protein of the coronavirus, the QTNSPRRRAR (SEQ ID NO: 14) sequence at position 677-685 of SEQ ID NO: x is replaced by the sequence QTILR (SEQ ID NO: 15).

In particular embodiments, if both the complete S1 subunit and the complete S2 subunit of the coronavirus are present, the SI/S2 cleavage site is deleted.

In particular embodiments, the Spike protein of the coronavirus or part thereof does not comprise the signal peptide of the coronavirus. In particular embodiments, the polypeptide of the multimeric protein complex as taught herein or the Spike protein of the coronavirus or part thereof does not comprise an amino acid sequence MFVFLVLLPLVS (SEQ ID NO: 22).

In particular embodiments, the polypeptides of the multimeric protein complex as taught herein comprise prior to multimerization a signal peptide. The signal peptide is typically cleaved off when the monomeric strands are within the endoplasmic reticulum. Multimerization takes place thereafter.

In particular embodiments, the signal peptide comprises. consists essentially of or consists of an amino acid sequence MGAGATGRAMDGPRLLLLLLLGVSLGGA (SEQ ID NO: 23). The signal peptide as defined by SEQ ID NO: 23 is the signal peptide of tumour necrosis factor receptor superfamily member 16 (UniProt Nr P08138.1). Present inventors found that the use of such signal peptide allows a good expression/secretion of the recombinant fusion polypeptides or multimeric protein complexes as taught herein in eukaryotic systems.

One or more restriction sites (e.g. the NheI and EcoRI restriction sites) and/or a Kozak sequence may be included N-terminally of the signal peptide. Accordingly, in particular embodiments, the polypeptides of the multimeric protein complex as taught herein comprise (prior to multimerization) N-terminally of the signal peptide a sequence LANSPVAA (SEQ ID NO: 24).

The multimerization domain comprising the collagen-like region (CLR) of ficolin or a ficolin-like protein enables the assembly of the polypeptides into a multimeric protein complex as taught herein, and hence, can therefore be considered a multimerization scaffold. Ficolin or ficolin-like proteins are naturally secreted proteins and therefore the use of the CLR of ficolin or ficolin-like proteins as multimerization scaffold per se or as substantial part of a multimerization scaffold for multimeric protein complexes is advantageous over the use of any multimeric fusion proteins that would have to be made intracellularly, and therefore not only may fold incorrectly for naturally secreted proteins, such as soluble receptors, but also make purification of such fusion proteins more difficult. A further advantage of using the CLR of ficolin or ficolin-like proteins as (part of) a multimerization scaffold for multimeric protein complexes over non-human protein multimerization domains such as from yeast, bacteria phage or bacteria is its low immunogenicity in the human body.

In particular embodiments, the multimerization domain comprises, consists essentially of or consists of CLR of ficolin or the ficolin-like protein, preferably the CLR of ficolin.

In particular embodiments, the multimerization domain comprises, consists essentially of or consists of the CLR of ficolin, wherein ficolin is selected from the group consisting of ficolin-2 (e.g. human L-ficolin or rat ficolin-B), ficolin-1 (e.g. human M-ficolin or rat ficolin-A) and ficolin-3, preferably ficolin is ficolin-2. In particular embodiments, ficolin is rat or human ficolin, preferably human ficolin. In particular embodiments, the multimerization domain comprises, consists essentially of or consists of the CLR of human ficolin-2. In particular embodiments, the multimerization domain consists of the CLR of human ficolin-2.

For example, the human ficolin-2 protein sequence is annotated under NCBI Genbank accession number NP_004099.2 (isoform a precursor) or NP_0566652.1 (isoform b precursor), and Uniprot (www.uniprot.org) accession number Q15485.2.

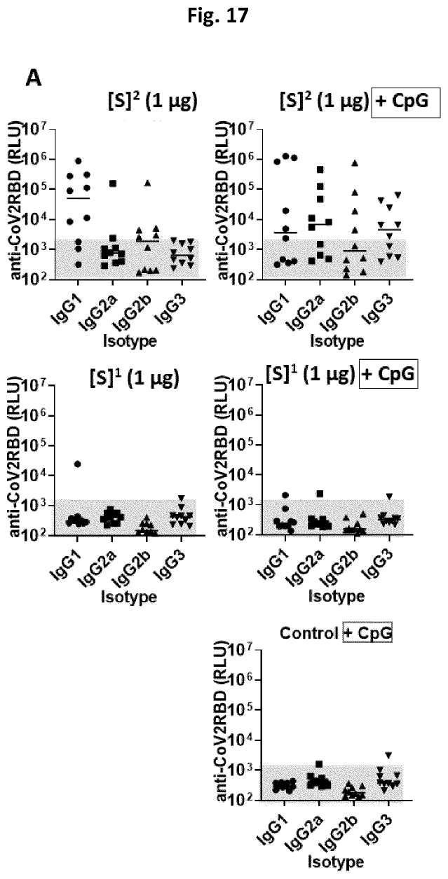

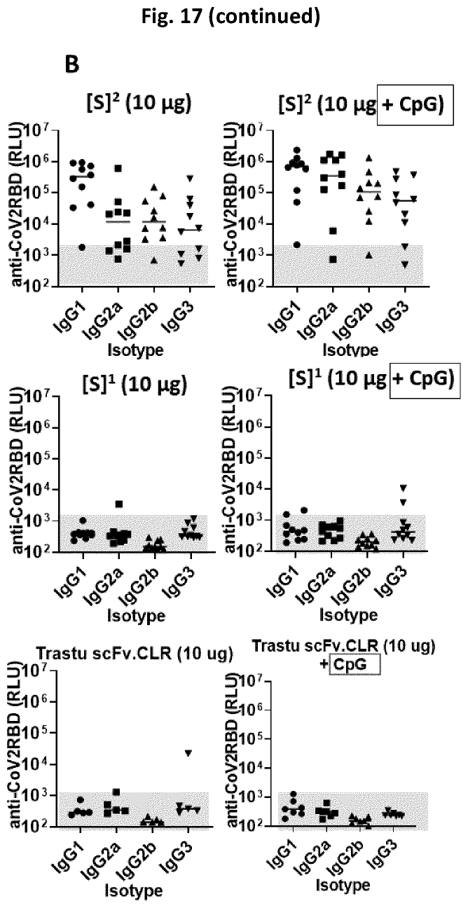

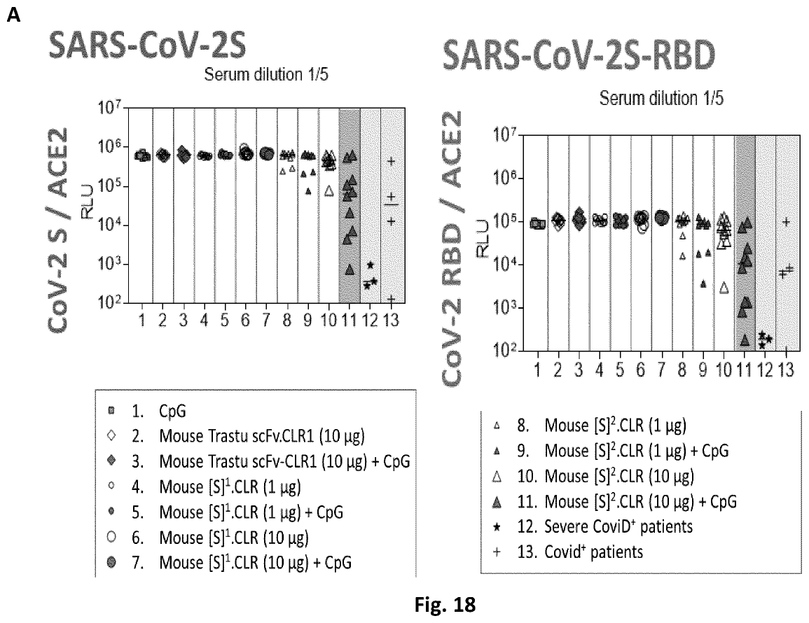

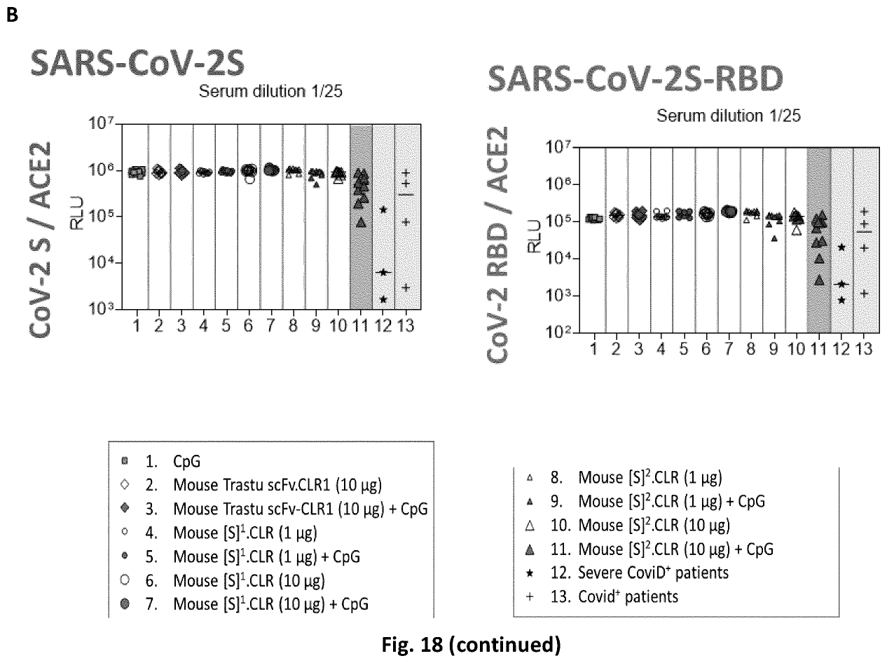

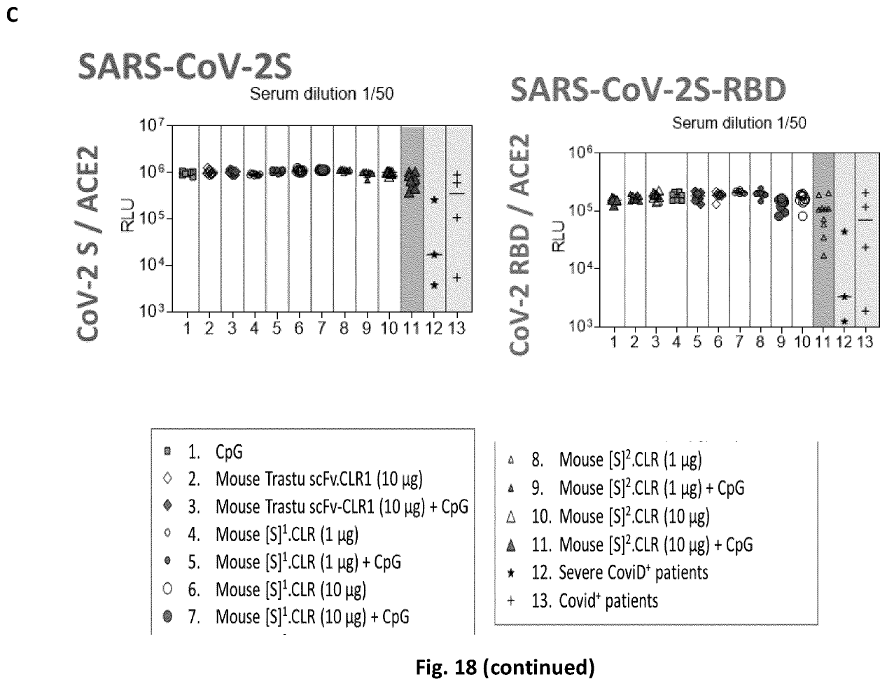

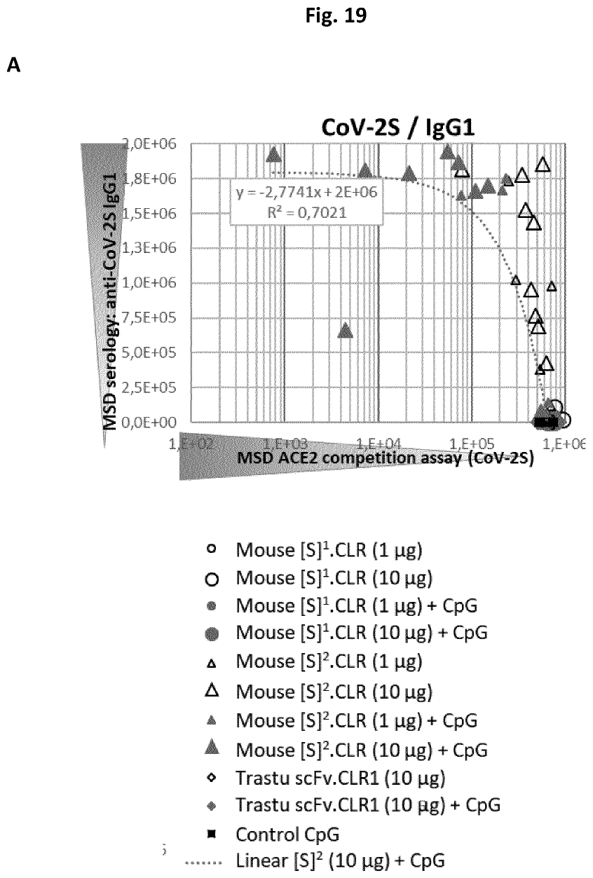

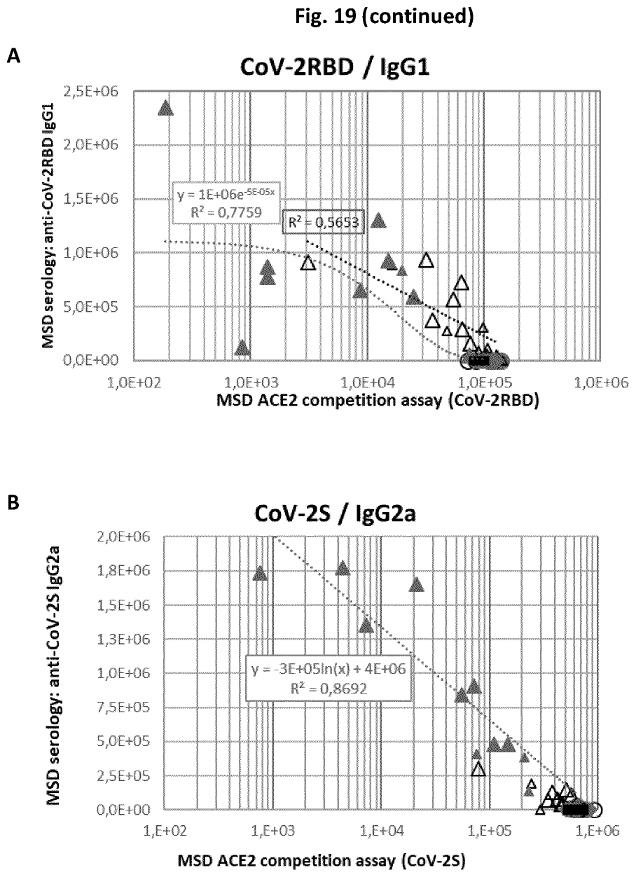

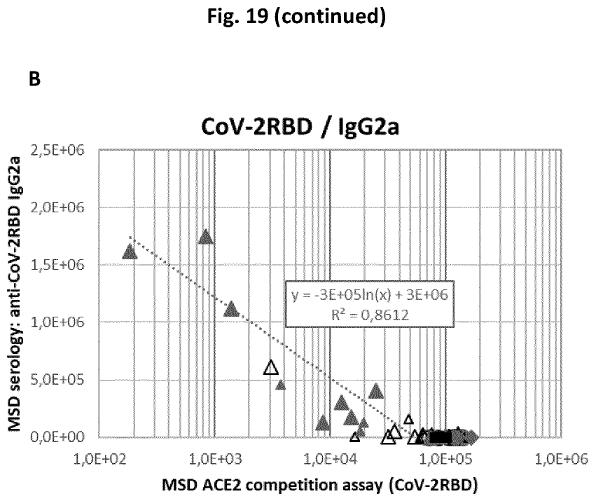

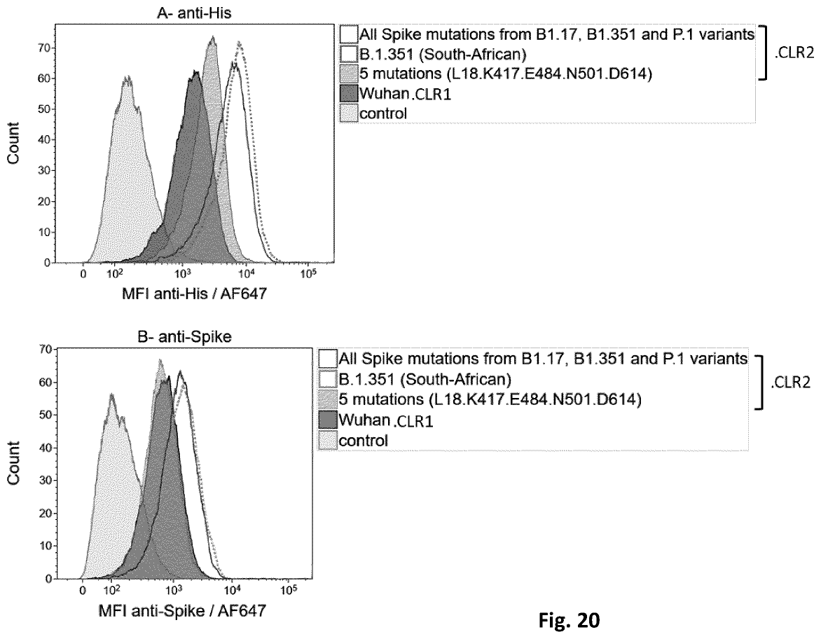

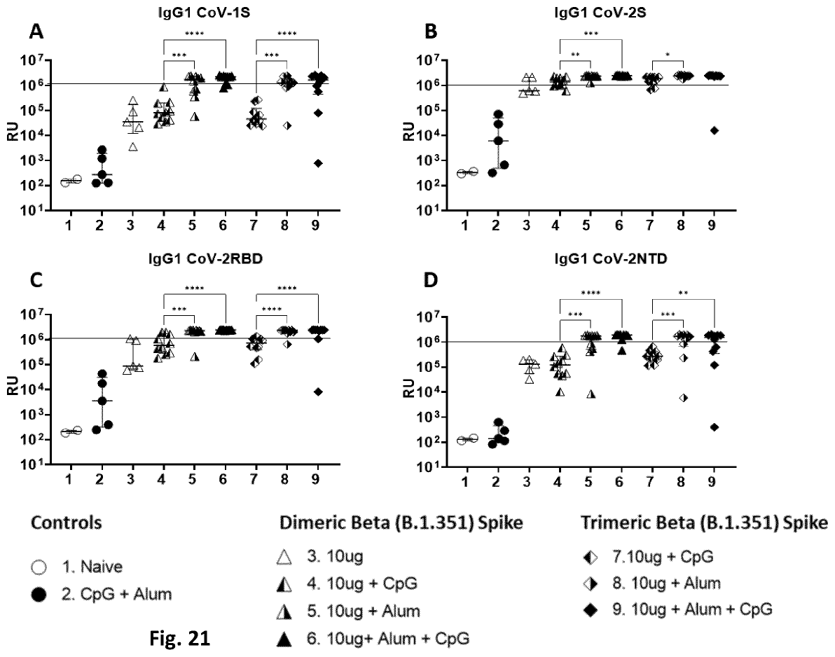

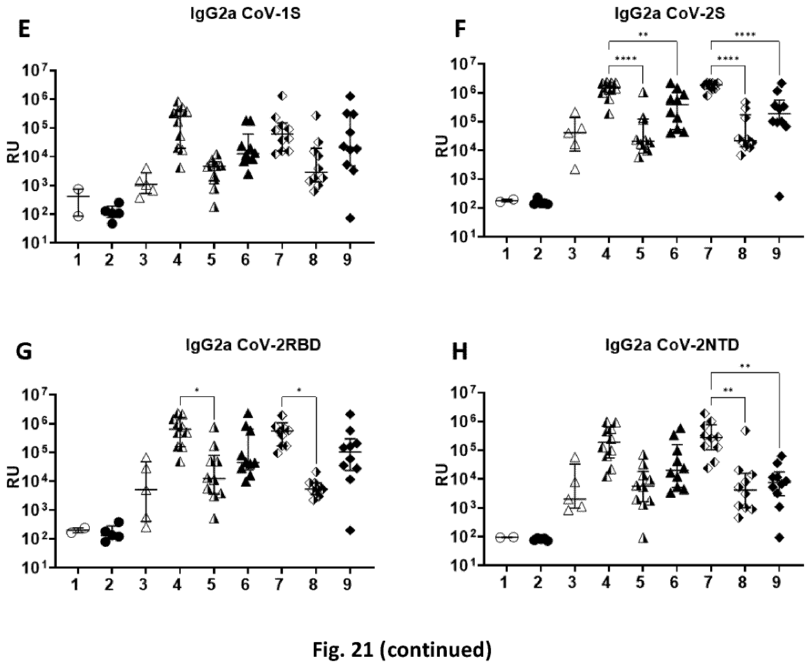

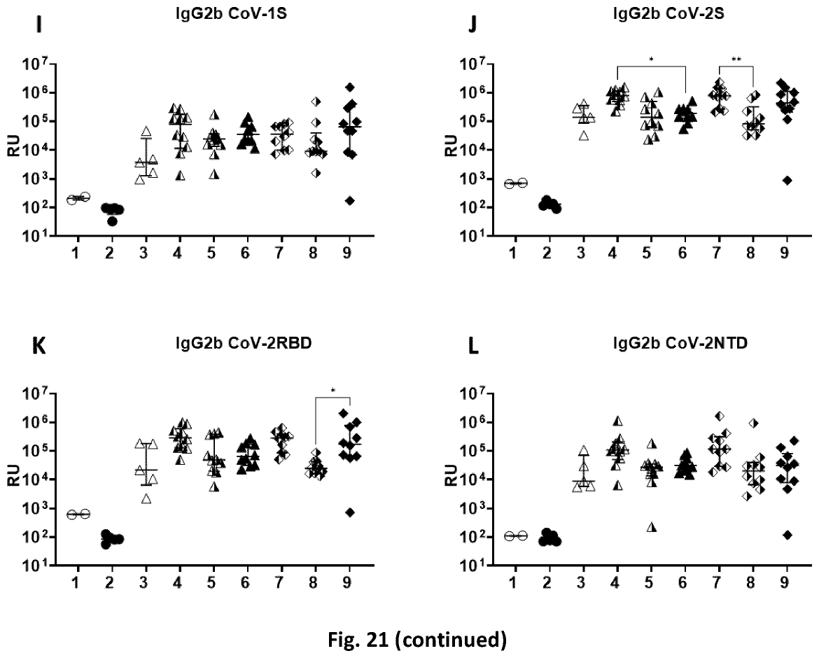

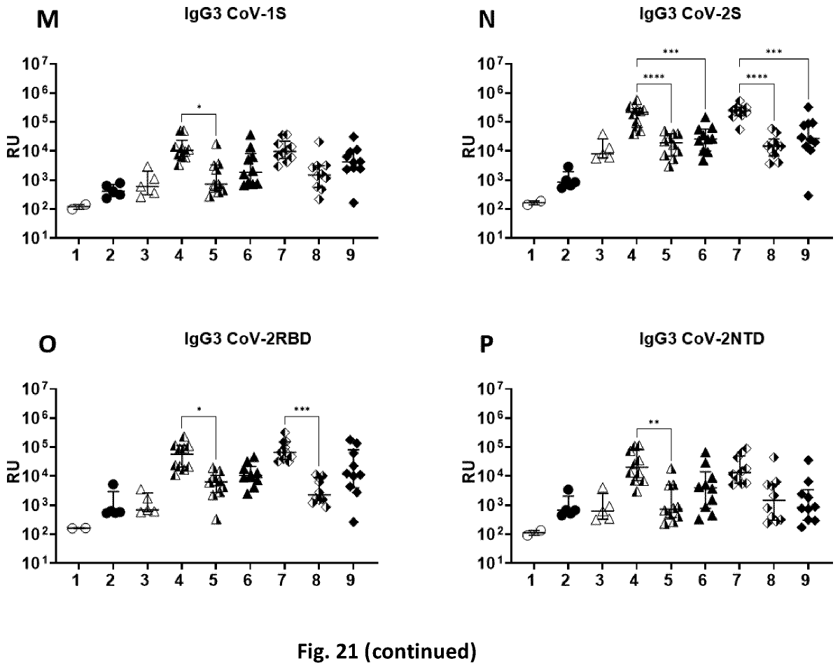

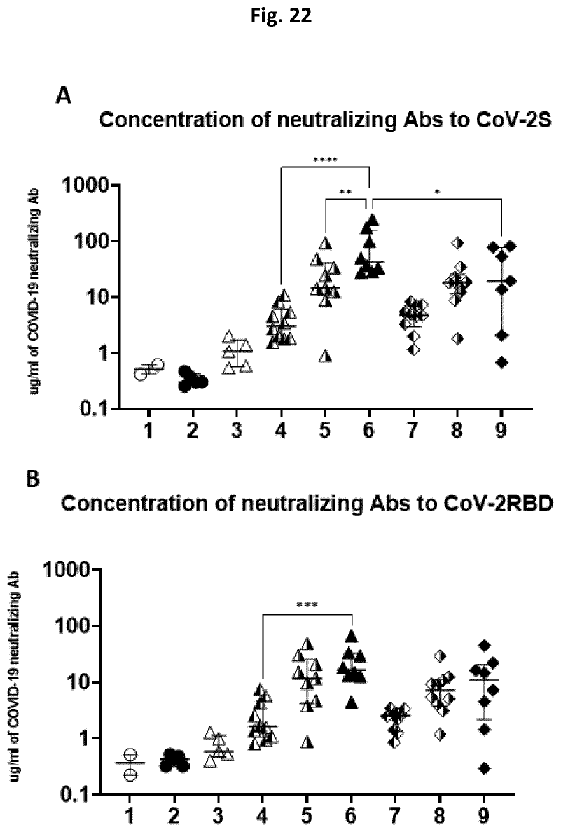

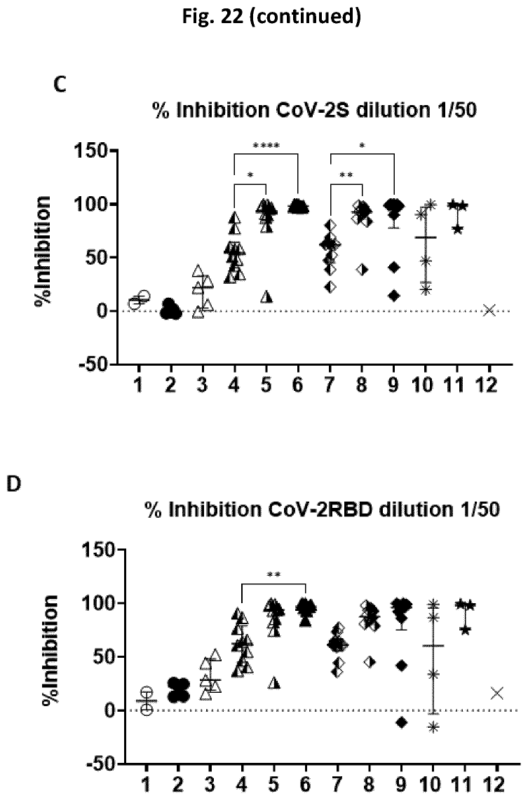

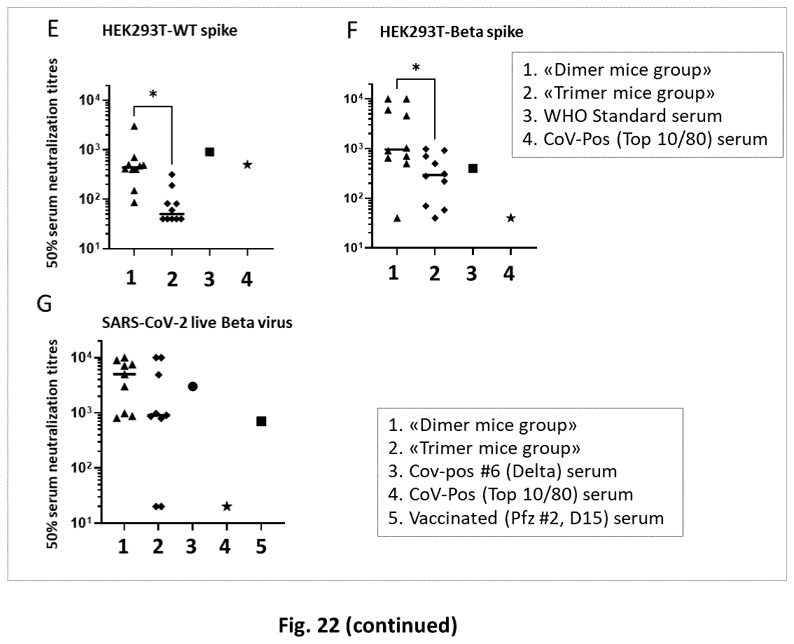

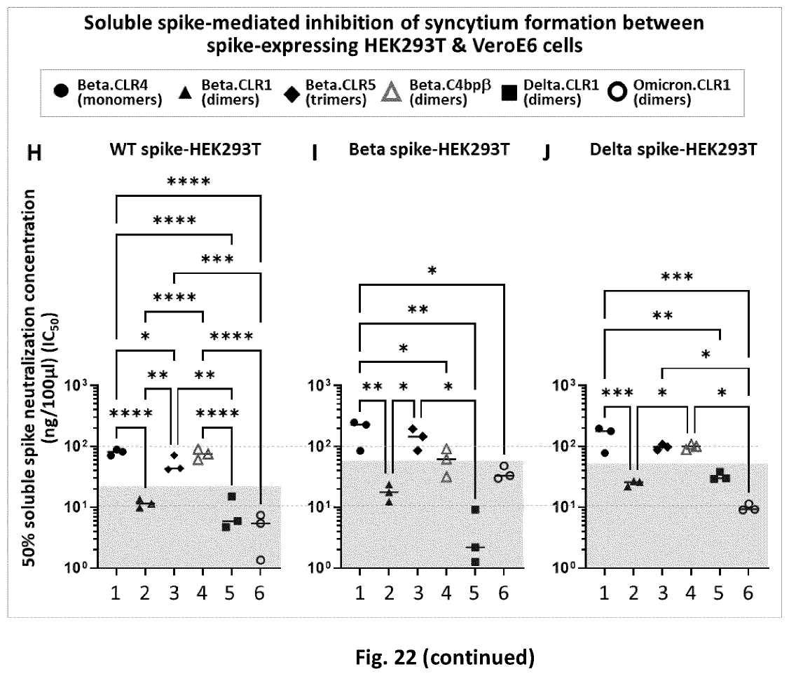

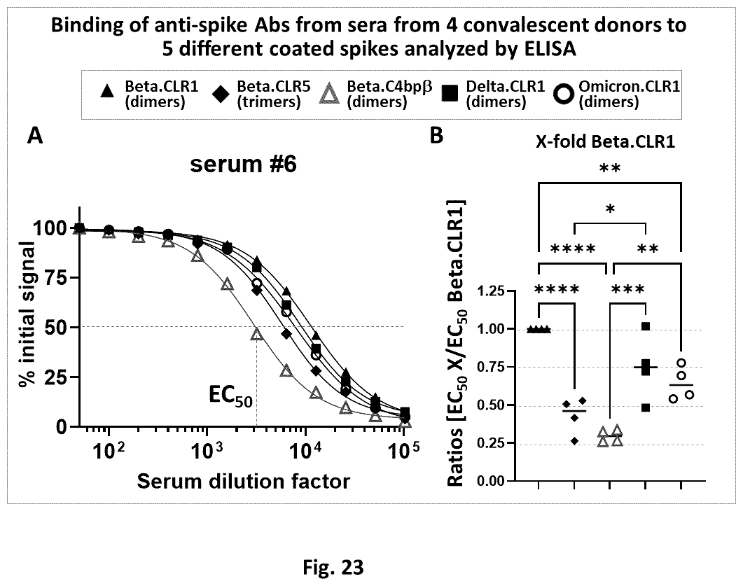

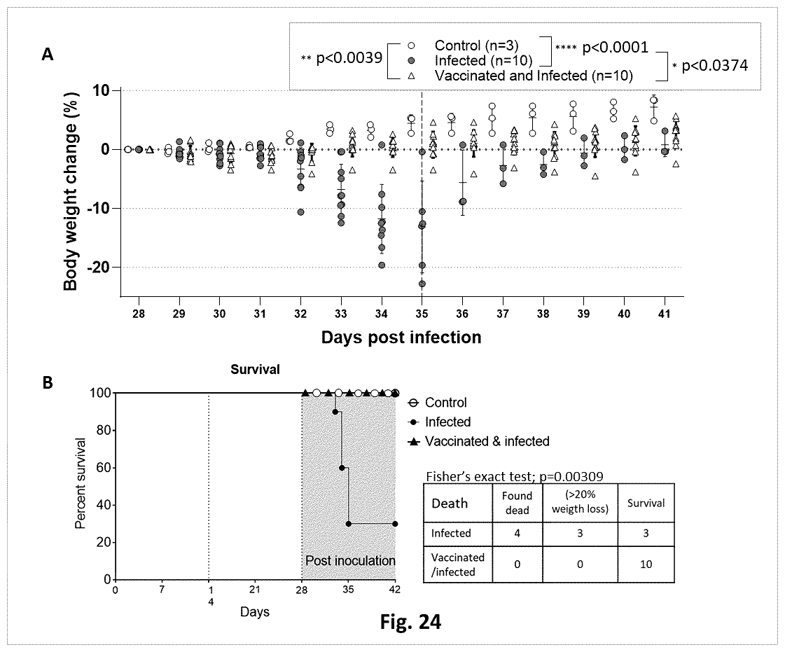

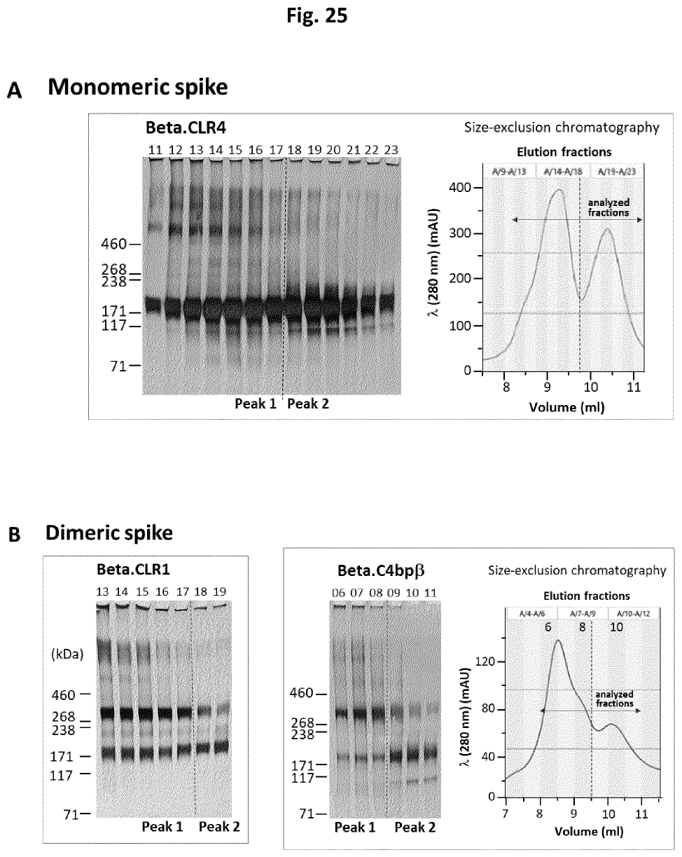

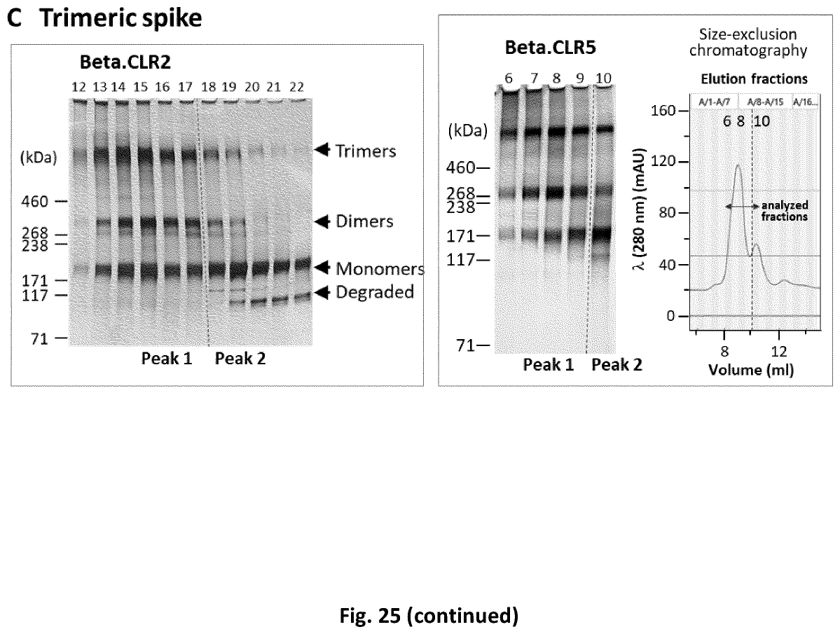

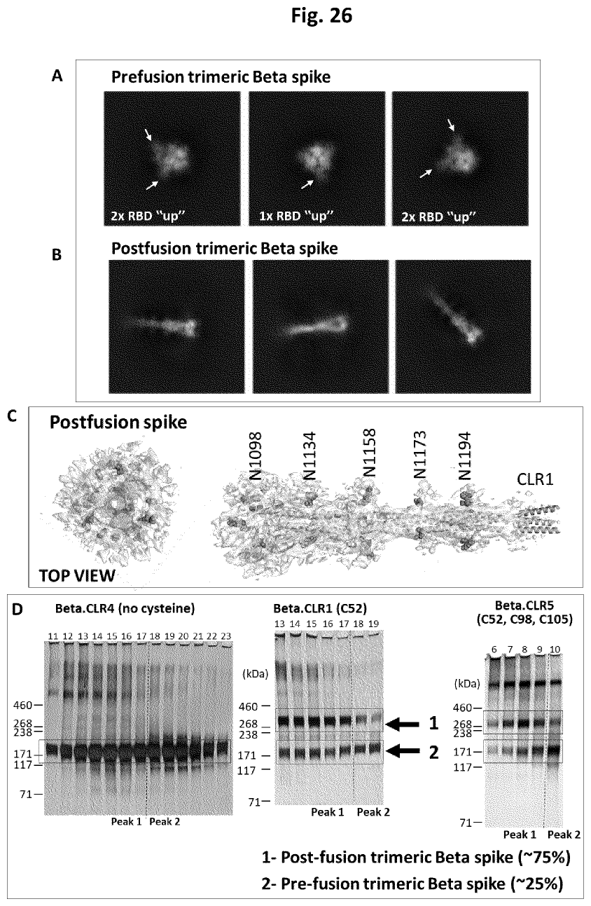

The precursor form of human ficolin-2 comprises N- to C-terminally a 25 amino acid signal peptide, a 25 amino acid cysteine-rich region (amino acids 26 to 50), a 45 amino acid collagen-like region (CLR) (amino acids 51 to 95) and a 218 amino acid fibrinogen-like region (FLR) (amino acids 96 to 313). The 25 amino acid N-terminal region of Ficolin-2—located upstream of the CLR—is called “Cysteine-rich region” and contains the Cysteine C32 and is not part of the collagen-like region (CLR). The CLR of human ficolin-2 is characterized by a repetition of fifteen G-X-Y sequences, wherein X can be any amino acid, allowing the triple-stranded coiled-coil association that characterizes a trimeric subunit. The Cysteine-rich region of ficolin-2 does not contain any G-X-Y repeats. The X in the first G-X-Y repeat of CLR is a cysteine, referred to in the present specification as “cysteine 52” or “C52”, as it is located at amino acid position 52 of the precursor form of human ficolin-2 as annotated under Uniprot (www.uniprot.org) accession number Q15485.2. The further cysteines of human ficolin-2 are named in a similar manner. The cysteines at positions 32 (C32) and 52 (C52) of the precursor form of human-ficolin-2 typically allow the covalent association of four ficolin-2 subunits into dodecamers (see for example FIG. 2 C-D) through the interchain disulfide formation of the C52 of one subunit with the C52 of another subunit. The FLR region of human ficolin-2 comprises at amino acid positions 98 (C98), 105 (C105), 126 (C126), 133 (C133), 257 (C257) and 270 (C270) cysteine residues. The sequence of human ficolin-2 without the 25 amino acid signal peptide is also referred to in the present specification as the sequence of mature human ficolin-2. In particular embodiments, the multimerization domain comprises the CLR of a ficolin-like protein, such as mannose-binding lectin.

Present inventors observed that trimeric protein complexes wherein the multimerization domain comprises a single cysteine, such as wherein the multimerization domain consists of the CLR of human ficolin-2 (e.g. “CLR1” as described elsewhere in the specification), lead to the formation of primarily pre-fusion stabilized trimeric spike glycoproteins (which are also referred to in the examples as “monomers”) and post-fusion stabilized trimeric spike glycoproteins (which are also referred to in the examples as “dimers”).

Furthermore, present inventors realized that the introduction of a second cysteine in the multimerization domain, preferably at the C-terminal end of the CLR, lead to the formation of substantial fractions of pre-fusion stabilized trimeric spike glycoproteins, post-fusion stabilized trimeric spike glycoproteins and presumed oligomers of the trimeric postfusion-stabilized spikes

Finally, present inventors found that the absence of any cysteine in the multimerization domain exclusively results in the formation of a single molecular species consisting of pre-fusion stabilized trimeric spike glycoprotein.

The introduction of a second cysteine in the multimerization domain can be achieved in several ways. For example, one of the amino acid residues of the native CLR sequence of ficolin or a ficolin-like protein used may be mutated into a cysteine. Preferably, the multimerization domain may consist of amino acids 51-98 of human ficolin-2 (e.g. “CLR2” as described elsewhere in the specification). These fragments of human ficolin-2 comprise by nature two cysteines in their amino acid sequence. For example, the multimerization domain may also consist of amino acids 51-105, amino acids 26-105, amino acids 51-126, or amino acids 51-133 of human ficolin-2, but not limited thereto, in which one or more cysteine residues are mutated to a non-cysteine residue in order to achieve a total of two cysteine residues in the multimerization domain. The multimerization domain comprising the CLR of ficolin or a ficolin-like protein can further also comprise a third, fourth, fifth or sixth cysteine. For example, two or more of the amino acid residues of the native CLR sequence used may be mutated into a cysteine. For example, to achieve a total amount of cysteines of three, the multimerization domain may consist of amino acids 51-105 of human ficolin-2, or amino acids 26-98 of human ficolin-2. These fragments of human ficolin-2 comprise by nature three cysteines in their amino acid sequence. For example, the multimerization domain may also consist of amino acids 26-105, amino acids 51-126, or amino acids 51-133 of human ficolin-2, but not limited thereto, in which one or more cysteine residues are mutated to a non-cysteine residue in order to achieve a total of three cysteine residues in the multimerization domain. The use of fragments of the native sequence of ficolin or a ficolin-like protein allows to reduce the risk of immunogenicity of the multimers as taught herein.

In particular embodiments, the multimerization domain comprises at least one or at least two cysteine residues. In particular embodiments, the multimerization domain comprises from 1 to 7, from 1 to 6, from 1 to 5, from 1 to 4, from 1 to 3, from 1 to 2 (i.e. one or two), from 2 to 7, from 2 to 6, from 2 to 5, or from 2 to 3 (i.e. two or three) cysteine residues, preferably from 1 to 3 cysteine residues, more preferably from 1 to 2 cysteine residues, even more preferably 1 cysteine residue. In particular embodiments, the multimerization domain comprises at most two cysteine residues, such as at most one cysteine residue.

In particular embodiments, the multimerization domain comprises, comprises, consists essentially of or consists of at most 15 [GXX] repeats, wherein X is C, L, A, P, E, T, or K.

In particular embodiments, the multimerization domain comprises, comprises, consists essentially of or consists of at most 15 consecutive [GXX] repeats, wherein X is C, L, A, P, E, T, or K, and wherein the first [GXX] repeat of the at most 15 consecutive [GXX] repeats comprises a cysteine residue, preferably wherein the first [GXX] repeat of the at most 15 consecutive [GXX] repeats consists of a sequence [GCX], wherein X is L, A, P, E, T, or K.

In particular embodiments, the multimerization domain comprises, comprises, consists essentially of or consists of 15 consecutive [GXX] repeats, wherein X is C, L, A, P, E, T, or K, and wherein the first [GXX] repeat (i.e. the most N-terminal repeat) comprises a sequence [GCX], wherein X is L, A, P, E, T, or K, and wherein C-terminally of said 15 consecutive [GXX] repeats there is a sequence XXC, wherein is L, A, P, E, T, or K.

In particular embodiments, the multimerization domain comprises a MASP-2 binding site. As a result thereof, the multimeric protein complex as taught herein has an increased adjuvanticity/immune reactivity by complement activation.

In particular embodiments, the multimerization domain comprises, comprises, consists essentially of or consists of 15 consecutive [GXX] repeats, wherein X is C, L, A, P, E, T, or K, and wherein the eleventh [GXX] repeat comprises a sequence [GKX], wherein X is L, A, P, E, T, or K, preferably a sequence [GKA].

The at most 15 consecutive [GXX] repeats, wherein X is C, L, A, P, E, T, or K, as well as the MASP-2 binding site are typically part of the CLR of ficolin or the ficolin-like protein within the multimerization domain.

Present inventors have found that the CLR of ficolin or the ficolin-like protein, preferably the CLR of ficolin-2, is sufficient to induce trimerization into a trimeric protein complex as taught herein. As a result of the limited size of the multimerization domain as taught herein, the resulting multimeric protein complex is less heavy and is closer to the native sequence of the antigen of the pathogen.

In particular embodiments, the multimerization domain as taught herein comprises, consists essentially of or consists of at most 75 amino acids, at most 73 amino acids, at most 70 amino acids, at most 65 amino acids, at most 60 amino acids, at most 55 amino acids, at most 50 amino acids, such as at most 49 amino acids, preferably at most 48 amino acids, such as at most 47 amino acids, at most 46 amino acids, more preferably at most 45 amino acids.

In particular embodiments, the multimerization domain comprises, consists essentially of or consists of an amino acid sequence having at least 70%, at least 80%, at least 90%, at least 95%, such as at least 96%, at least 97%, at least 98%, or at least 99%, preferably 100%, sequence identity with a sequence GCPGLPGAPGPKGEAGTNGKRGERGPPGPPGKAGPPGPNGAPGEP (SEQ ID NO: 25: “CLR1”), wherein there is a cysteine residue at position 2 of SEQ ID NO: 25.

In particular embodiments, the multimerization domain comprises, consists essentially of or consists of (i) CLR of ficolin or the ficolin-like protein, preferably the CLR of ficolin-2, and
(ii) a peptide consisting of at most 35 amino acids, preferably at most 30 amino acids, more preferably at most 25 amino acids, N-and/or C-terminally of the CLR, wherein said peptide comprises at least one, preferably from 1 to 3, more preferably one, cysteine residues.

In particular embodiments, the multimerization domain comprises, consists essentially of or consists of (i) CLR of ficolin or the ficolin-like protein, preferably the CLR of ficolin-2, and
(ii) a peptide consisting of at most 35 amino acids, preferably at most 30 amino acids, more preferably at most 25 amino acids, N-terminally of the CLR, wherein said amino acid sequence comprises one cysteine residue, preferably wherein said peptide is the cysteine-rich region of ficolin, more preferably the cysteine-rich region of ficolin-2.

In particular embodiments, the multimerization domain comprises, consists essentially of or consists of (i) CLR of ficolin or the ficolin-like protein, preferably the CLR of ficolin-2, and
(ii) a peptide consisting of at most 35 amino acids, preferably at most 10 amino acids, more preferably at most 5 amino acids, even more preferably at most 3 amino acids, C-terminally of the CLR, wherein said amino acid sequence comprises at least one, preferably from 1 to 3, more preferably one, cysteine residue, preferably wherein said amino acid sequence is a fragment of the FLR region of ficolin, preferably of ficolin-2.

In particular embodiments, the multimerization domain comprises, consists essentially of or consists of (i) CLR of ficolin or the ficolin-like protein, preferably the CLR of ficolin-2,
(ii) a peptide consisting of at most 35 amino acids, preferably at most 30 amino acids, more preferably at most 25 amino acids, N-terminally of the CLR, wherein said amino acid sequence comprises one cysteine residue, preferably wherein said peptide is the cysteine-rich region of ficolin, more preferably the cysteine-rich region of ficolin-2, and
(iii) a peptide consisting of at most 35 amino acids, preferably at most 10 amino acids, more preferably at most 5 amino acids, even more preferably at most 3 amino acids, C-terminally of the CLR, wherein said amino acid sequence comprises at least one, preferably from 1 to 3, more preferably one, cysteine residue, preferably wherein said amino acid sequence is a fragment of the FLR region of ficolin, preferably of ficolin-2.

In particular embodiments, the polypeptides of the multimeric protein complex as taught herein comprises. consists essentially of or consists of an amino acid sequence having at least 90%, at least 95%, such as at least 96%, at least 97%, at least 98%, or at least 99%, preferably 100%, sequence identity with
a sequence GCPGLPGAPGPKGEAGTNGKRGERGPPGPPGKAGPPGPNGAPGEPQPC (SEQ ID NO: 26: “CLR2”), wherein there is only a cysteine residue at position 2 and position 48 of SEQ ID NO: 26 (i.e. and no cysteine residue at the other positions of SEQ ID NO: 26);
a sequence LQAADTCPEVKMVGLEGSDKLTILRGCPGLPGAPGPKGEAGTNGKRGERGPPGPPGKAGPPGPN GAPGEP (SEQ ID NO: 27, “CLR3”), wherein there is only a cysteine residue at position 7 and position 27 of SEQ ID NO: 27 (i.e. and no cysteine residue at the other positions of SEQ ID NO: 27); or
a sequence GCPGLPGAPGPKGEAGTNGKRGERGPPGPPGKAGPPGPNGAPGEPQPCLTGPRTC (SEQ ID NO: 28, “CLR5”), wherein there is only a cysteine residue at position 2, position 48 and/or position 55 of SEQ ID NO: 28 (i.e. and no cysteine residue at the other positions of SEQ ID NO: 28). In particular embodiments. the polypeptides of the multimeric protein complex as taught herein comprises a fragment of the fibrinogen-like region (FLR) of ficolin, preferably of the fibrinogen-like region of ficolin-2, more preferably of human ficolin-2, of at most 50 amino acids, at most 40 amino acids, at most 35 amino acids, at most 30 amino acids, at most 25 amino acids, at most 20 amino acids, at most 15 amino acids, at most 10 amino acids, at most 5 amino acids, or at most 3 amino acids, preferably at most 10 amino acids or at most 3 amino acids.

In particular embodiments, the polypeptides of the multimeric protein complex as taught herein do not comprise an amino acid sequence having at least 90%, at least 95%, such as at least 96%, at least 97%, at least 98%, or at least 99%, preferably 100%, sequence identity with SEQ ID NO: 29. The sequence as defined by SEQ ID NO: 29 corresponds to the fibrinogen-like region of human ficolin-2. In particular embodiments, the polypeptides of the multimeric protein complex as taught herein do not comprise the cysteine-rich region of ficolin, preferably of the fibrinogen-like region of ficolin-2. In more particular embodiments, the polypeptide of the trimeric protein complex as taught herein do not comprise the first 25 amino acids of mature ficolin, preferably mature ficolin-2, more preferably mature human ficolin-2. In particular embodiments, the polypeptides of the multimeric protein complex as taught herein do not comprise an amino acid sequence having at least 90%, at least 95%, such as at least 96%, at least 97%, at least 98%, or at least 99%, preferably 100%, sequence identity with LQAADTCPEVKMVGLEGSDKLTILR (SEQ ID NO: 30). The sequence as defined by SEQ ID NO: 30 corresponds to the N-terminal region of human ficolin-2.

In particular embodiments, the polypeptides of the multimeric protein complex as taught herein each comprise a linker peptide C-terminally of the antigen of the pathogen, such as C-terminally of the S2 subunit of the S protein of the coronavirus, and N-terminally of the CLR of ficolin or the ficolin-like protein, preferably the CLR of ficolin-2.

As used herein, the term “linker” refers to a connecting clement that serves to link other elements. The linker may be a rigid linker (also referred to in the present specification as a spacer) or a flexible linker. In particular embodiments, the linker is a covalent linker, achieving a covalent bond. The terms “covalent” or “covalent bond” refer to a chemical bond that involves the sharing of one or more electron pairs between two atoms. For many molecules, the sharing of electrons allows each atom to attain the equivalent of a full outer electron shell, corresponding to a stable electronic configuration. Covalent bonds include different types of interactions, including σ-bonds, π-bonds, metal-to-metal bonds, agnostic interactions, bent bonds and three-center two-electron bonds.

The peptide linker may be 1 to 50 amino acids long or 2 to 50 amino acids long or 1 to 45 amino acids long or 2 to 45 amino acids long, preferably I to 40 amino acids long or 2 to 40 amino acids long or 1 to 35 amino acids long or 2 to 35 amino acids long, more preferably 1 to 30 amino acids long or 2 to 30 amino acids long. Further preferably, the linker may be 5 to 25 amino acids long or 5 to 20 amino acids long. Particularly preferably, the linker may be 5 to 15 amino acids long or 7 to 15 amino acids long. Hence, in certain embodiments, the linker may be 1, 2, 3 or 4 amino acids long. In other embodiments, the linker may be 5, 6, 7, 8 or 9 amino acids long. In further embodiments, the linker may be 10, 11, 12, 13 or 14 amino acids long. In still other embodiments, the linker may be 15, 16, 17, 18 or 19 amino acids long. In further embodiments, the linker may be 20, 21, 22, 23, 24 or 25 amino acids long. In certain embodiments, the linker is 4-10 or 5-9 or 6-8 or 7 amino acids long. In other embodiments, the linker is 12-18 or 13-17 or 14-16 or 15 amino acids long.

The nature of amino acids constituting the linker is not of particular relevance so long as the biological activity of the polypeptide segments linked thereby is not substantially impaired and the linker provides for the intended spatial separation of the C-terminal fragment of the C4bp beta-chain and a functional component. Preferred linkers are essentially non-immunogenic and/or not prone to proteolytic cleavage.

In certain preferred embodiments, the peptide linker may comprise, consist essentially of or consist of amino acids selected from the group consisting of Glycine, Serine, Alanine, Threonine, and combinations thereof. In even more preferred embodiments, the linker may comprise, consist essentially of or consist of amino acids selected from the group consisting of Glycine, Serine, and combinations thereof. Such linkers provide for particularly good flexibility. In certain embodiments, the linker may consist of only Glycine residues. In certain embodiments, the linker may consist of only Serine residues.

In particular embodiments, the linker is a flexible linker comprising, consisting essentially of or consisting of an amino acid sequence SGGGGS (SEQ ID NO: 1), 3×(SGGGGS) (SEQ ID NO: 31), or 5×(SGGGGS) (SEQ ID NO: 32), preferably SGGGGS (SEQ ID NO: 1).

In particular embodiments, the linker is a spacer comprising, consisting essentially of or consisting of an amino acid sequence RDCDPPGNPVHGYFEGNNFTLGSTISYYCEDRYYLVGVQEQQCVDGEWSSALPVCKL (SEQ ID NO: 33). Such spacer corresponds to the short consensus repeat 3 (SCR3) from the C4bp beta-chain which displays 4 cysteine residues folded by the presence of two internal disulphide bridges. The SCR3 has no biological function and is the natural spacer of the C4bp beta-chain to move the first two SCRs away from each other to the dimerization scaffold.

In particular embodiments, at least one, preferably all, of the polypeptides of the multimeric protein complex as taught herein comprises at its C-terminal end a tag.

A tag can be attached to proteins for various purposes, such as purification (e.g poly (His) tag), to assist proper protein folding (e.g. thioredoxin), separation techniques (e.g. FLAG-tag), enzymatic or chemical modifications (e.g. biotin ligase tags, FIAsH), or detection (e.g. tracking or visualization). Tags for detection can typically be visualized either directly or indirectly through detection with a labeled antibody or other protein or molecule binding or interacting with the tag. Examples of such tags are AviTag, Calmodulin-tag, polyglutamate tag, E-tag, FLAG-tag, HA-tag, His-tag, Myc-tag, S-tag, SBP-tag, Softag 1, Softag 3, Strep tag, TC tag, V5 tag, VSV-tag, Xpress tag, Isopeptag, SpyTag, Biotin Carboxyl Carrier Protein, Glutathione-S-transferase-tag, Green fluorescent protein tag, Halo-tag, Maltose binding protein-tag, Nus-tag, Thioredoxin-tag or Fc-tag, but is not limited thereto. The term “tag” as used herein also encompasses other tracking components such as a fluorescent protein (eGFP, eRFP, Cherry), a magnetic bead, biotin for staining with labelled avidin or streptavidin conjugate, an enzyme, a substrate, a cofactor, a chemiluminescent group (e.g. nanoluciferase), a chromogenic agent, a colorimetric label, a molecular imaging probe (e.g. ¹⁸F, ¹¹C, or ⁶⁴Cu, ^{99m}Tc, iron oxide nanoparticles, or luciferase).

Preferably the tag is a peptide, protein or polypeptide. In particular embodiments, the tag is a peptide, protein or polypeptide having an amino acid sequence of at most 10 amino acids. In particular embodiments, the tag is a protein purification tag or a protein separation tag. More preferably, the tag is a FLAG-tag, His-tag, HA-tag or Myc-tag. Even more preferably, the tag is a His 8×-tag.

In particular embodiments, the tag comprises N-terminally a proteolytic cleavage site.

Example of such cleavage sites are well known in the art and include a Tobacco Etch Virus (TEV) protease cleavable site, such as comprising an amino acid sequence ENLYFQ/G (SEQ ID NO: 34), or a Human rhinovirus (HRV) 3C protease cleavable site, such as comprising an amino acid sequence LEVLFQ/GP (SEQ ID NO: 35), wherein ‘/’ represents the peptide bond which will be cleaved), as are the methods to introduce them in the constructs of the invention or to use them for releasing protein moieties.

Preferably, the proteolytic cleavage site is a TEF protease cleavable site, such as comprising an amino acid sequence ENLYFQ/G (SEQ ID NO: 34), or a HRV 3C protease cleavable site, such as comprising an amino acid sequence LEVLFQ/GP (SEQ ID NO: 35), more preferably a HRV 3C protease cleavable site.

In particular embodiments, the polypeptides each comprise C-terminally the amino acid sequence LEVLFQGBHHHHHHHH (SEQ ID NO: 36).

In particular embodiments, the polypeptides of the multimeric protein complex as taught herein comprise one or more proteins or polypeptides C-terminally of the CLR of ficolin or ficolin-like proteins, preferably ficolin-2.

In particular embodiments, the polypeptides of the multimeric protein complex as taught herein do not comprise a fibrogen-like region of ficolin or ficolin-like protein, and comprise one or more proteins or polypeptides C-terminally of the CLR of ficolin or ficolin-like protein, preferably ficolin-2.

The introduction of anti-SARS-CoV-2 T cell response activators in the polypeptides of the multimeric protein complex allows generating a vaccine that is able to boost the anti-spike antibody response, but also the T-cell response.

Accordingly, in particular embodiments, the protein or polypeptide located C-terminally of the CLR is a selective activator of the anti-coronavirus T-cell response, preferably the anti-SARS-CoV-2 T cell response, more preferably the anti-SARS-CoV-2 CD8 T cell response. In particular embodiments, the protein or polypeptide located C-terminally of the CLR is capable of selectively activating cytotoxic T lymphocytes and helper T lymphocytes. Non-limiting examples of such proteins or polypeptides are SARS-CoV-2 open reading frames (ORFs), such as ORF1 (e.g. ORF1a, ORFab), Spike (S), ORF3 (e.g. ORF3a, ORF3b), envelope (E), membrane (M), nucleocapsid (N), ORF5, OFR6, ORF7a, ORF7b, ORF8 (e.g. ORF8a, ORF8b), ORF9, ORF10, or a combination thereof, or fragments thereof, preferably SARS-CoV-2 M, E, N, ORF1, ORF2, ORF3, ORF5, ORF8, ORF9, or a combination thereof, such as described in Nelde A. et al., SARS-CoV-2-derived peptides define heterologous and COVID-19-induced T cell recognition, Nature Immunology, 2021, 22: 74-85; Obaidullah A J et al., Immunoinformatics-guided design of a multi-epitope vaccine based on the structural proteins of severe acute respiratory syndrome coronavirus 2, RSV Adv., 2021, 11, 18103; or Le Bert N. et al., SARS-CoV-2-specific T cell immunity in cases of COVID-19 and SARS, and uninfected controls, Nature, 2020, 584:457-462. The ORFs may also be referred to as non-structural proteins (NSPs).

In particular embodiments, all polypeptides of the multimeric protein complex are identical.

A further aspect provides a polynucleotide encoding a polypeptide of the multimeric protein complex as taught herein.

The term “nucleic acid” as used throughout this specification typically refers to a polymer (preferably a linear polymer) of any length composed essentially of nucleoside units. A nucleoside unit commonly includes a heterocyclic base and a sugar group. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups (such as without limitation 2′-O-alkylated, e.g., 2′-O-methylated or 2′-O-ethylated sugars such as ribose; 2′-O-alkyloxyalkylated, e.g., 2′-O-methoxyethylated sugars such as ribose; or 2′-O,4′-C-alkylene-linked, e.g., 2′-O,4′-C-methylene-linked or 2′-O,4′-C-ethylene-linked sugars such as ribose; 2′-fluoro-arabinose, etc.). Nucleic acid molecules comprising at least one ribonucleoside unit may be typically referred to as ribonucleic acids or RNA. Such ribonucleoside unit(s) comprise a 2′-OH moiety, wherein —H may be substituted as known in the art for ribonucleosides (e.g., by a methyl, ethyl, alkyl, or alkyloxyalkyl). Preferably, ribonucleic acids or RNA may be composed primarily of ribonucleoside units, for example, ≥80%, ≥85%, ≥90%, ≥95%, ≥96%, ≥97%, ≥98%, ≥99% or even 100% (by number) of nucleoside units constituting the nucleic acid molecule may be ribonucleoside units. Nucleic acid molecules comprising at least one deoxyribonucleoside unit may be typically referred to as deoxyribonucleic acids or DNA. Such deoxyribonucleoside unit(s) comprise 2′-H, Preferably, deoxyribonucleic acids or DNA may be composed primarily of deoxyribonucleoside units, for example, ≥80%, ≥85%, ≥90%, ≥95%, ≥96%, ≥97%, ≥98%, ≥99% or even 100% (by number) of nucleoside units constituting the nucleic acid molecule may be deoxyribonucleoside units, Nucleoside units may be linked to one another by any one of numerous known inter-nucleoside linkages, including inter alia phosphodiester linkages common in naturally-occurring nucleic acids, and further modified phosphate- or phosphonate-based linkages such as phosphorothioate, alkyl phosphorothioate such as methyl phosphorothioate, phosphorodithioate, alkylphosphonate such as methylphosphonate, alkylphosphonothioate, phosphotriester such as alkylphosphotriester, phosphoramidate, phosphoropiperazidate, phosphoromorpholidate, bridged phosphoramidate, bridged methylene phosphonate, bridged phosphorothioate; and further siloxane, carbonate, sulfamate, carboalkoxy, acetamidate, carbamate such as 3′-N-carbamate, morpholino, borano, thioether, 3′-thioacetal, and sulfone internucleoside linkages. Preferably, inter-nucleoside linkages may be phosphate-based linkages including modified phosphate-based linkages, such as more preferably phosphodiester, phosphorothioate or phosphorodithioate linkages or combinations thereof.

The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids, RNA is inclusive of dsRNA (double stranded RNA), mRNA (messenger RNA), miRNA (micro-RNA), tRNA (transfer RNA, whether charged or discharged with a corresponding acylated amino acid), and cRNA (complementary RNA). A nucleic acid can be naturally occurring, e.g., present in or isolated from nature, e.g., produced natively or endogenously by a cell or a tissue and optionally isolated therefrom. A nucleic acid can be recombinant, i.e., produced by recombinant DNA technology, and/or can be, partly or entirely, chemically or biochemically synthesised. Without limitation, a nucleic acid can be produced recombinantly by a suitable host or host cell expression system and optionally isolated therefrom (e.g., a suitable bacterial, yeast, fungal, plant or animal host or host cell expression system), or produced recombinantly by cell-free transcription, or non-biological nucleic acid synthesis. A nucleic acid can be double-stranded, partly double stranded, or single-stranded. Where single-stranded, the nucleic acid can be the sense strand or the antisense strand. In addition, nucleic acid can be circular or linear.

In particular embodiments, the nucleotide sequence consecutively encodes the S1 and S2 subunit of the coronavirus Spike protein. The skilled person will understand that this means that the sequence encoding the S1 subunit is located 5′ of the sequence encoding the S2 subunit. The nucleotide sequence consecutively encoding the S1 and S2 subunit will typically comprise a S1/S2 cleavage site formed by the 3′ end of the S1 subunit and the 5′ end of the S2 subunit of the coronavirus Spike protein. As described elsewhere in the present specification, this S1/S2 cleavage site may be mutated to prevent proteolytic processing of the S protein in the S1 and S2 subunits.

In particular embodiments, the polynucleotide as taught herein comprises the full-length sequence of the precursor form (i.e. including the full length signal peptide or a part thereof) of the coronavirus spike protein.

In particular embodiments, the polynucleotide as taught herein does not comprise the nucleotide sequence encoding the signal peptide or part of the signal peptide of the coronavirus Spike protein. The signal peptide of a coronavirus Spike protein typically comprises, consists essentially of or consists of 36 nucleotides (encoding 12 amino acids). Accordingly, the nucleotide sequence encoding the signal peptide or part of the signal peptide of a coronavirus Spike protein may comprise from 1 to 36 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 nucleotides.

A further aspect provides a nucleic acid expression cassette comprising the polynucleotide as taught herein, optionally linked to a promoter and/or transcriptional and translational regulatory signals.

The term “nucleic acid expression cassettes” as used herein refers to nucleic acid molecules, typically DNA, to which nucleic acid fragments, preferably the recombinant nucleic acid molecule as defined herein, may be inserted to be expressed, wherein said nucleic acid molecules comprise one or more nucleic acid sequences controlling the expression of the nucleic acid fragments. Non-limiting examples of such more nucleic acid sequences controlling the expression of the nucleic acid fragments include promoter sequences, open reading frames and transcription terminators. An “open reading frame” or “ORF” refers to a succession of coding nucleotide triplets (codons) starting with a translation initiation codon and closing with a translation termination codon known per se, and not containing any internal in-frame translation termination codon, and potentially capable of encoding a protein, polypeptide or peptide. Hence, the term may be synonymous with “coding sequence” as used in the art. An “operable linkage” is a linkage in which regulatory sequences and sequences sought to be expressed are connected in such a way as to permit said expression. For example, sequences, such as, e.g., a promoter and an ORF, may be said to be operably linked if the nature of the linkage between said sequences does not: (1) result in the introduction of a frame-shift mutation, (2) interfere with the ability of the promoter to direct the transcription of the ORF, (3) interfere with the ability of the ORF to be transcribed from the promoter sequence. Hence, “operably linked” may mean incorporated into a genetic construct so that expression control sequences, such as a promoter, effectively control transcription/expression of a sequence of interest.

The precise nature of transcriptional and translational regulatory sequences or elements required for expression may vary between expression environments, but typically include a transcription terminator, and optionally an enhancer.

Reference to a “promoter” is to be taken in its broadest context and includes transcriptional regulatory sequences required for accurate transcription initiation and where applicable accurate spatial and/or temporal control of gene expression or its response to, e.g., internal or extrernal (e.g., exogenous) stimuli. More particularly, “promoter” may depict a region on a nucleic acid molecule, preferably DNA molecule, to which an RNA polymerase binds and initiates transcription. A promoter is preferably, but not necessarily, positioned upstream, i.e., 5′, of the sequence the transcription of which it controls. Typically, in prokaryotes a promoter region may contain both the promoter per se and sequences which, when transcribed into RNA, will signal the initiation of protein synthesis (e.g., Shine-Dalgamo sequence). A promoter sequence can also include “enhancer regions”, which are one or more regions of DNA that can be bound with proteins (namely the trans-acting factors) to enhance transcription levels of genes in a gene-cluster. The enhancer, while typically at the 5′ end of a coding region, can also be separate from a promoter sequence, e.g., can be within an intronic region of a gene or 3′ to the coding region of the gene.

The terms “terminator” or “transcription terminator” refer generally to a sequence element at the end of a transcriptional unit which signals termination of transcription. For example, a terminator is usually positioned downstream of, i.e., 3′ of ORF(s) encoding a polypeptide of interest, For instance, where a recombinant nucleic acid contains two or more ORFs, e.g., successively ordered and forming together a multi-cistronic transcription unit, a transcription terminator may be advantageously positioned 3′ to the most downstream ORF.

In particular embodiments, the nucleic acid expression cassette comprises the polynucleotide as taught herein, operably linked to one or more promoters, enhancers, ORFs and/or transcription terminators.

A further aspect provides an expression vector comprising the polynucleotide as taught herein.

The terms “expression vector” or “vector” as used herein refers to nucleic acid molecules, typically DNA, to which nucleic acid fragments, preferably the recombinant nucleic acid molecule as defined herein, may be inserted and cloned, i.e., propagated. Hence, a vector will typically contain one or more unique restriction sites, and may be capable of autonomous replication in a defined cell or vehicle organism such that the cloned sequence is reproducible. A vector may also preferably contain a selection marker, such as, e.g., an antibiotic resistance gene, to allow selection of recipient cells that contain the vector. Vectors may include, without limitation, plasmids, phagemids, bacteriophages, bacteriophage-derived vectors, PAC, BAC, linear nucleic acids, e.g., linear DNA, transposons, viral vectors, etc., as appropriate (sec, e.g., Sambrook et al., 1989; Ausubel 1992). Viral vectors may include inter alia retroviral vectors, lentiviral vectors, adenoviral vectors, or adeno-associated viral vectors, for example, vectors based on HIV, SV40, EBV, HSV or BPV. Expression vectors are generally configured to allow for and/or effect the expression of nucleic acids or open reading frames introduced thereto in a desired expression system, e.g., in vitro, in a cell, organ and/or organism. For example, expression vectors may advantageously comprise suitable regulatory sequences.

Factors of importance in selecting a particular vector include inter alia: choice of recipient cell, case with which recipient cells that contain the vector may be recognised and selected from those recipient cells which do not contain the vector; the number of copies of the vector which are desired in particular recipient cells; whether it is desired for the vector to integrate into the chromosome or to remain extra-chromosomal in the recipient cells; and whether it is desirable to be able to “shuttle” the vector between recipient cells of different species.

Expression vectors can be autonomous or integrative. A nucleic acid can be in introduced into a cell in the form of an expression vector such as a plasmid, phage, transposon, cosmid or virus particle. The recombinant nucleic acid can be maintained extrachromosomally or it can be integrated into the cell chromosomal DNA. Expression vectors can contain selection marker genes encoding proteins required for cell viability under selected conditions (e.g., URA3, which encodes an enzyme necessary for uracil biosynthesis, or LEU2, which encodes an enzyme required for leucine biosynthesis, or TRP1, which encodes an enzyme required for tryptophan biosynthesis) to permit detection and/or selection of those cells transformed with the desired nucleic acids. Expression vectors can also include an autonomous replication sequence (ARS). The ARS may comprise a centromere (CEN) and an origin of replication (ORI), For example, the ARS may be ARS18 or ARS68.

Prior to introducing the vectors into a cell of interest, the vectors can be grown (e.g., amplified) in bacterial cells such as Escherichia coli (E. coli). The vector DNA can be isolated from bacterial cells by any of the methods known in the art, which result in the purification of vector DNA from the bacterial milieu. The purified vector DNA can be extracted extensively with phenol, chloroform, and ether, to ensure that no E. coli proteins are present in the plasmid DNA preparation, since these proteins can be toxic to mammalian cells.

The polypeptides of the multimeric protein complex as taught herein may be suitably obtained through expression by host cells or host organisms, transformed with an expression construct encoding and configured for expression of said polypeptides in said host cells or host organisms, followed by purification of the protein, polypeptide or peptide.

Hence, a further aspect provides a host cell comprising the polynucleotide, the nucleic acid expression cassette or vector as taught herein.

In certain embodiments, the host cell may be a mammalian cell.

The polynucleotide or the multimeric protein complex as taught herein may be suitably isolated. The term “isolated” with reference to a particular component (such as for instance a nucleic acid, protein, polypeptide or peptide) generally denotes that such component exists in separation from—for example, has been separated from or prepared and/or maintained in separation from—one or more other components of its natural environment. For instance, an isolated human or animal protein or complex may exist in separation from a human or animal body where it naturally occurs. The term “isolated” as used herein may preferably also encompass the qualifier “purified”. As used herein, the term “purified” with reference to peptides, polypeptides, proteins, or nucleic acids does not require absolute purity. Instead, it denotes that such peptides, polypeptides, proteins, or nucleic acids are in a discrete environment in which their abundance (conveniently expressed in terms of mass or weight or concentration) relative to other analytes is greater than in the starting composition or sample. A discrete environment denotes a single medium, such as for example a single solution, gel, precipitate, lyophilisate, etc. Purified nucleic acids, proteins, polypeptides or peptides may be obtained by known methods including, for example, laboratory or recombinant synthesis, chromatography, preparative electrophoresis, centrifugation, precipitation, affinity purification, etc. Purified peptides, polypeptides or proteins may preferably constitute by weight ≥10%, more preferably ≥50%, such as ≥60%, yet more preferably ≥70%, such as ≥80%, and still more preferably ≥90%, such as ≥95%, ≥96%, ≥97%, ≥98%, >99% or even 100%, of the protein content of the discrete environment. Protein content may be determined, e.g., by the Lowry method (Lowry et al. 1951, J Biol Chem 193: 265), optionally as described by Hartree 1972 (Anal Biochem 48: 422-427). Purity of peptides, polypeptides, or proteins may be determined by SDS-PAGE under reducing or non-reducing conditions using Coomassie blue or, preferably, silver stain. Quantity of nucleic acids may be determined by measuring absorbance A260. Purity of nucleic acids may be determined by measuring absorbance A260/A280, or by agarose- or polyacrylamide-gel electrophoresis and ethidium bromide or similar staining.

Further, there are several other well-known methods of introducing nucleic acids into animal cells, any of which may be used herein. At the simplest, the nucleic acid can be directly injected into the target cell/target tissue. Other methods include fusion of the recipient cell with bacterial protoplasts containing the nucleic acid, the use of compositions like calcium chloride, rubidium chloride, lithium chloride, calcium phosphate, DEAE dextran, cationic lipids or liposomes or methods like receptor-mediated endocytosis, biolistic particle bombardment (“gene gun” method), infection with viral vectors (i.e. derived from lentivirus, adeno-associated virus (AAV), adenovirus, retrovirus or antiviruses), electroporation, and the like. Other techniques or methods which are suitable for delivering nucleic acid molecules to target cells include the continuous delivery of an NA molecule from poly (lactic-Co-Glycolic Acid) polymeric microspheres or the direct injection of protected (stabilized) NA molecule(s) into micropumps delivering the product. Another possibility is the use of implantable drug-releasing biodegradable microspheres. Also envisaged is encapsulation of NA in various types of liposomes (immunoliposomes, PEGylated (immuno) liposomes), cationic lipids and polymers, nanoparticles or dendrimers, poly (lactic-Co-Glycolic Acid) polymeric microspheres, implantable drug-releasing biodegradable microspheres, etc.; and co-injection of NA with protective agent like the nuclease inhibitor aurintricarboxylic acid. It shall be clear that also a combination of different above-mentioned delivery modes or methods may be used.

In particular embodiments, the expression vector is suitable for the expression of recombinant proteins in eukaryotic cells. In particular embodiments, the expression vector is a pEF-IRESpac vector, such as described in Hobbs S., et al., Development of a bicistronic vector driven by the human polypeptide chain elongation factor 1 alpha promoter for creation of stable mammalian cell lines that express very high levels of recombinant proteins, Biochem Biophys Res Commun., 1998, 252(2):368-72.

A further aspect provides a pharmaceutical composition comprising the polypeptide as taught herein, the multimeric protein complex as taught herein, the polynucleotide as taught herein or the expression vector as taught herein, and a pharmaceutically acceptable carrier.

A further aspect provides a composition, such as the pharmaceutical composition as described herein, comprising a mixture of different trimeric protein complexes as taught herein, such as a mixture of pre-fusion stabilized trimeric spike (which are also referred to in the examples as “monomers”) and post-fusion stabilized trimeric spike (which are also referred to in the examples as “dimers”), of post-fusion stabilized trimeric spike and presumed oligomers of the trimeric post-fusion-stabilized spikes (which are also referred to in the examples as “trimers”), or of presumed oligomers of the trimeric post-fusion-stabilized spikes, post-fusion stabilized trimeric spike and presumed oligomers of the trimeric post-fusion-stabilized spikes.

Accordingly, a further aspect provides a composition comprising a combination of protein complexes, the protein complexes comprising one, two or three, preferably three polypeptides, each comprising N- to C-terminally: (i) a RBD of an S1 subunit of an S protein of a coronavirus, (ii) optionally a S2 subunit of an S protein of a coronavirus: and (iii) a multimerization domain comprising a CLR of ficolin-2, wherein the polypeptides have not assembled (i.e. also referred to in the present specification as monomeric protein complexes or monomers), the polypeptides have assembled into dimeric protein complexes (i.e. dimers) by way of said multimerization domain or the polypeptides have assembled into trimeric protein complexes (i.e. trimers). In particular embodiments, composition comprising a combination of protein complexes, the protein complexes comprising three polypeptides, each comprising N- to C-terminally:
(i) a RBD of an S1 subunit of an S protein of a coronavirus,
(ii) optionally a S2 subunit of an S protein of a coronavirus; and
(iii) a multimerization domain comprising a CLR of ficolin-2, wherein the polypeptides have not assembled, or the polypeptides have assembled into trimeric protein complexes by way of said multimerization domain.

In particular embodiments, the total amount of pre-fusion stabilized trimeric spike glycoproteins (also referred to in the examples as monomeric protein complexes) present in the composition is at most 50%, at most 40%, at most 35%, at most 30%, at most 25%, at most 20%, at most 15%, at most 10%, at most 5%, such as at most 4%, at most 3%, at most 2% or at most 1%, preferably at most 35%, of the total amount of protein complexes (i.e. pre-fusion stabilized trimeric spike glycoproteins, post-fusion stabilized trimeric spike glycoproteins and presumed oligomers of the trimeric post-fusion-stabilized spikes) as taught herein present in the composition. In particular embodiments, the composition comprises post-fusion stabilized trimeric spike glycoproteins and/or presumed oligomers of the trimeric post-fusion-stabilized spikes as taught herein, but no or substantially no non-oligomerized post-fusion stabilized trimeric spike glycoproteins as taught herein.

In particular embodiments, the total amount of post-fusion stabilized trimeric spike glycoproteins and/or presumed oligomers of the trimeric post-fusion-stabilized spikes present in the composition is at least 50%, at least 60%, at least 70%, at least 80%, at least 85%, at least 90%, at least 95%, such as at least 96%, at least 97%, at least 98%, at least 99% or 100%, of the total amount of protein complexes (i.e pre-fusion stabilized trimeric spike glycoproteins, post-fusion stabilized trimeric spike glycoproteins and presumed oligomers of the trimeric post-fusion-stabilized spikes) as taught herein present in the composition.

In particular embodiments, the total amount of post-fusion stabilized trimeric spike glycoproteins in the composition is at least 50%, at least 60%, at least 70%, at least 80%, at least 85%, at least 90%, at least 95%, such as at least 96%, at least 97%, at least 98%, at least 99% or 100%, of the total amount of protein complexes (e.g. pre-fusion stabilized trimeric spike glycoproteins, post-fusion stabilized trimeric spike glycoproteins and presumed oligomers of the trimeric post-fusion-stabilized spikes) as taught herein present in the composition.

In particular embodiments,
(i) the total amount of post-fusion stabilized trimeric spike glycoproteins in the composition is at least 70%, at least 75%, at least 80%, at least 85%, at least 90%, at least 95%, such as at least 96%, at least 97%, at least 98%, at least 99% or 100%, preferably at least 75%, of the total amount of protein complexes (e.g. pre-fusion stabilized trimeric spike glycoproteins, post-fusion stabilized trimeric spike glycoproteins and/or presumed oligomers of the trimeric post-fusion-stabilized spikes) as taught herein present in the composition; and
(ii) the total amount of pre-fusion stabilized trimeric spike glycoproteins in the composition is at most 50%, at most 40%, at most 35%, at most 30%, at most 25%, at most 20%, at most 15%, at most 10%, at most 5%, such as at most 4%, at most 3%, at most 2% or at most 1%, preferably at most 25%, of the total amount of protein complexes (e.g. pre-fusion stabilized trimeric spike glycoproteins, post-fusion stabilized trimeric spike glycoproteins and/or presumed oligomers of the trimeric post-fusion-stabilized spikes) as taught herein present in the composition.

The term “pharmaceutically acceptable” as used herein is consistent with the art and means compatible with the other ingredients of a pharmaceutical composition and not deleterious to the recipient thereof.

“Acceptable carrier, diluent or excipient” refers to an additional substance that is acceptable for use in human and/or veterinary medicine, with particular regard to vaccines.

By way of example, an acceptable carrier, diluent or excipient may be a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulphate and carbonates, vegetable oils, synthetic oils, polyols, alginic acid, phosphate buffered solutions, emulsifiers, isotonic saline and salts such as mineral acid salts including hydrochlorides, bromides and sulphates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, (1991)) which is incorporated herein by reference.

Any safe route of administration may be employed for providing a patient with the vaccine, For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Intra-muscular and subcutaneous injection may be appropriate, for example, for administration of proteinaceous vaccines and nucleic acid vaccines.

In particular embodiments, the pharmaceutical composition as taught herein is formulated for intramuscular injection.

In particular embodiment, the multimeric protein complex or pharmaceutical composition as taught herein can be stored at room temperature, such as at a temperature of about 4° C., for a period of at least 1 day, at least 2 days, at least 3 days, at least 4 days, at least 5 days, at least 6 days or at least 7 days.

In particular embodiment, the multimeric protein complex or the pharmaceutical composition as taught herein can be stored at fridge temperature, such as at a temperature of about −20° C., for a period of at least 1 week, at least 2 weeks, at least 1 month, or at least 2 months.

In particular embodiment, the multimeric protein complex or the pharmaceutical composition as taught herein can be stored at fridge temperature, such as at a temperature of about −80° C., for a period of at least 1 months, at least 2 months, at least 3 months, at least 4 months, at least 5 months or at least 6 months.

In particular embodiments, the multimeric protein complex might be stored at −80° C., or −20° C., before being dispersed into vaccine vials.

A further aspect provides the multimeric protein complex as taught herein, the polynucleotide as taught herein, the composition as taught herein, or the pharmaceutical composition as taught herein for use as a medicament.

In particular embodiments, the medicament is a vaccine. Accordingly the invention provides the use of the multimeric protein complex as taught herein, the polynucleotide as taught herein, the composition as taught herein, or the pharmaceutical composition as taught herein in the manufacture of a medicament, more particularly in the manufacture of a vaccine.

In particular embodiments, the vaccine comprises one or more adjuvants. The use of adjuvants in vaccines is well known. An adjuvant is a compound that, when combined with a vaccine antigen, increases the immune response to the vaccine antigen as compared to the response induced by the vaccine antigen alone. Among strategies that promote antigen immunogenicity are those that render vaccine antigens particulate, those that polymerize or emulsify vaccine antigens, methods of encapsulating vaccine antigens, ways of increasing host innate cytokine responses, and methods that target vaccine antigens to antigen presenting cells, Conventional adjuvants, well-known in the art, are diverse in nature. They may, for example, consist of water-insoluble inorganic salts, liposomes, micelles or emulsions, i.e. Freund's adjuvant, alum, CpG oligonucleotides, polyA-polyU, dimethyldioctadecylammonium bromide (DDA), N,N-dioctadecyl-N′,N′-bis(2-hydroxyethyl)propanediamine, carbomer, or chitosan.

In particular embodiments, the vaccine comprises CpG, Alum, or a combination thereof.

CpGs are nucleotide sequences comprising a CpG motif (i.e. a sequence containing an unmethylated cytosine followed by guanosine and linked by a phosphate bond) and are well known in the art as adjuvants for vaccines, such as described in Scheiermann J, and Klinman D. M., Clinical evaluation of CpG oligonucleotides as adjuvants for vaccines targeting infectious diseases and cancer, Vaccine, 2014, 32(48):6377-6389 or Montamat G. et al., CpG adjuvant in allergen-specific immunotherapy: finding the sweet spot for the induction of immune tolerance, Front Immunol., 2021, 12:590054). CpGs can include nucleotide modifications/analogs such as phosphorothioate modifications and can be double stranded or single stranded. Optionally, guanosine can be replaced with an analog such as 2′-deoxy-7-desazaguanosine.

The CpG sequence can be directed to toll-like receptor 9 (TLR9), such as the GTCGTT or TTCGTT motif. The CpG sequence may be specific to induce a Th1 immune response, such as a CpG-A ODN, or it may be more specific to induce a B lymphocyte response, such as a CpG-B ODN.

Agonists of TLR9, TLR8, TLR8 and TLR3 confer a successful stimulation of the immune response in connection with the vaccine as taught herein as they stimulate the Type I interferon responses. Accordingly, in particular embodiments, the CpG is a TLR9, TLR7, TLR8 or TLR3 agonist.

In particular embodiments, the CpG is a B-type CpG.

Concentrations of one or more adjuvants, such as CpG, in a vaccine are known in the art, and might be 500 meg per 1 ml dose, such as described in Jackson et al., Immunogenicity of a two-dose investigational hepatitis B vaccine, HBsAg-1018, using a toll-like receptor 9 agonist adjuvant compared with a licensed hepatitis B vaccine in adults, Vaccine 36 (2018): 668-674.

In particular embodiments, the vaccine is a pan-coronavirus vaccine, such as a pan-SARS-CoV-2 vaccine.

A further aspect provides the polypeptide as taught herein, the multimeric protein complex as taught herein, the polynucleotide as taught herein, the composition or the pharmaceutical composition as taught herein for use in preventing an infection with a pathogen wherein the pathogen corresponds to the pathogen of which the antigen is included in the polypeptides of the multimeric protein complex as taught herein, preferably for use in preventing a coronavirus infection, more preferably a SARS-CoV-2 infection.

In particular embodiments, the SARS-CoV-2 infection may be an infection with any variant of the SARS-CoV-2 virus, such as the SARS-CoV-2 variants as described elsewhere herein. In particular embodiments, if the multimeric protein complex as taught herein comprises an S protein SARS-CoV-2 variant Beta or a fragment thereof, the SARS-CoV-2 infection to be treated is preferably a SARS-CoV-2 infection with the WT (Wuhan) and/or Beta SARS-CoV-2 variant.

In other words, provided herein is a method for preventing an infection with a pathogen, preferably for preventing a coronavirus infection (e.g. a method of vaccinating against a coronavirus), more preferably a SARS-CoV-2 infection, in a subject comprising administering a prophylactically effective amount of the polypeptide as taught herein, the multimeric protein complex as taught herein, the polynucleotide as taught herein, the composition as taught herein, or the pharmaceutical composition as taught herein. Except when noted, the terms “subject” or “patient” can be used interchangeably and refer to animals, preferably warm-blooded animals, more preferably vertebrates, even more preferably mammals, still more preferably primates, and specifically includes human patients and non-human mammals and primates. Preferred subjects are human subjects.

In particular embodiments, two doses of the multimeric protein complex as taught herein, or the pharmaceutical composition as taught herein are administered to the subject, preferably wherein the first and the second dose are at least two weeks apart.

In particular embodiments, a single dose comprises, consists essentially of or consists of from 5 μg to 50 μg, from 5 μg to 40 μg, from 5 μg to 30 μg, from 10 μg to 40 μg, from 20 μg to 40 μg or from 10 μg to 30 μ2 g, such as 30 μg of the polypeptide or the multimeric protein complex as taught herein.

In particular embodiments, the multimeric protein complex as taught herein or the pharmaceutical composition as taught herein are administered intramuscular.

A further aspect provides the use of the polypeptides and/or the multimeric protein complexes as taught herein for diagnostic purposes, such as in an in vitro method for determining the binding or neutralization activity of human serum antibodies after vaccination or after natural infection with SARS-CoV-2 towards the human receptor ACE2.

A further aspect provides in vitro method of preparing the multimeric protein complex, such as the multimeric protein complex as taught herein.

In particular embodiments, the method of preparing the multimeric protein complexes, such as the multimeric protein complexes as taught herein, comprises a two-step purification.

In particular embodiments, the method of preparing the multimeric protein complexes, such as the multimeric protein complexes as taught herein, comprises
(a) introducing a polynucleotide encoding a polypeptide comprising N- to C-terminally:
   (i) a receptor-binding domain (RBD) of an S1 subunit of a Spike (S) protein of a coronavirus,
   (ii) optionally a S2 subunit of an S protein of a coronavirus; and
   (iii) a multimerization domain comprising a collagen-like region (CLR) of ficolin-2, wherein the multimerization domain enables the assembly of the polypeptides into a trimeric protein complex, into a host cell,
(b) allowing the host cell to express and secrete the polypeptides, resulting in the self-multimerization of the polypeptides into trimeric protein complexes; and
(c) separating the trimeric protein complexes from the supernatants.

In particular embodiments, the method of preparing the multimeric protein complexes, such as the multimeric protein complexes as taught herein, comprises
(i) introducing the polynucleotide encoding the polypeptide of the multimeric protein complex as taught herein into a host cell,
(ii) allowing the host cell to express and secrete the polypeptide of the multimeric protein complexes as taught herein, resulting in the self-multimerization (i.e. spontaneous multimerization) of the polypeptides into multimeric protein complexes; and
(iii) separating the multimeric protein complexes as taught herein from the supernatants.

In particular embodiments, the method for preparing a trimeric protein complex is a method for stabilizing the post-fusion form of the covid spike protein, wherein the polynucleotide encodes a polypeptide comprises a multimerization domain comprising a collagen-like region (CLR) of ficolin-2 comprising 1 cysteine, preferably 1 cysteine. More preferably, wherein the multimerization domain comprises, consists essentially of or consists of an amino acid sequence having at least 70%, at least 80%, at least 90%, at least 95%, such as at least 96%, at least 97%, at least 98%, or at least 99%, preferably 100%, sequence identity with a sequence GCPGLPGAPGPKGEAGTNGKRGERGPPGPPGKAGPPGPNGAPGEP (SEQ ID NO: 25; “CLR1”), wherein there is a cysteine residue at position 2 of SEQ ID NO: 25.

Spontaneous multimerization typically takes place in the endoplasmic reticulum and Golgi in the reducing environment of these cell organelles of the cell machinery export compartments.

In particular embodiments, if the polynucleotide encoding the polypeptide of the multimeric protein complex as taught herein comprises an antibiotic resistance genes, the colony-forming cell clones (CFC) that are resistant to the antibiotic selection appear and start expanding.

In particular embodiments, the method of preparing the multimeric protein complexes as taught herein comprises a step of selecting (e.g. manually) individual colony-forming cell clones with the highest expression of the polypeptide of the multimeric protein complexes as taught herein and optionally further expanding said cell clones with the highest expression of the polypeptide of the multimeric protein complexes as taught herein.

The individual colony-forming cell clones with the highest expression may be selected by screening of supematants from individually picked-up cell clones using HEK293T/ACE2 commercial target cells. Supernatants from of individual colony-forming cell clones can be incubated with HEK293T/ACE2 cells (e.g. 150,000 cells/well). Then cells can be incubated with a rabbit anti-HIS antibody and a secondary antibody (goat anti-rabbit /AF647) and analysed using flow cytometry.

Further expanding the cell clones with the highest expression of the polypeptide of the multimeric protein complexes as taught herein may be performed in 500 ml cell culture medium in 5-chamber cell stacks.

In particular embodiments, the step of separating the multimeric protein complexes as taught herein from the supernatants comprises the steps of:
(iii.i) immobilizing the multimeric protein complexes by a tag comprised in the multimeric protein complexes, preferably a HIS-tag, using metal-ion affinity chromatography (IMAC),
(iii.ii) optionally cleaving the tag from the trimeric protein complexes,
(iii.iiii) eluting the multimeric protein complexes using an elution buffer, such as an elution buffer comprising imidazole and optionally concentrating the eluate, and
(iii.iii) optionally separating the multimeric protein complexes from the elution buffer, for example, using gel-filtration, Gel-filtration allows separating the imidazole and/or cleaved poly-his fragment from the multimeric protein complex.

The IMAC pre-purification step may be performed by all methods known in the art. The IMAC pre-purification step may comprise elution using stepwise imidazole gradients or a single imidazole elution step (e.g. using IM imidazole). For example, the IMAC pre-purification step may be performed using a 5 ml His-Trap Excel column for 5 days using a peristatic pump (e.g. flow-rate 2 to 3 ml/min). IMAC with stepwise imidazole gradient may allow to purify the multimeric protein complexes from the non-multimeric protein complexes contaminants and to separate the different molecular species of the multimeric protein complexes from each other (such as separating pre-fusion stabilized trimeric spike glycoproteins and post-fusion stabilized trimeric spike glycoproteins).

In particular embodiments, the method may comprise a step of separating the degraded forms of the multimeric protein complexes from the multimeric protein complexes using gel filtration (i.e. size-exclusion chromatography).

In particular embodiments, such as when a single elution step is used, the method may comprise a step of separating the pre-fusion stabilized trimeric spike glycoproteins complexes from the post-fusion stabilized trimeric spike glycoproteins and/or the presumed oligomers of the trimeric post-fusion-stabilized spikes, using gel filtration (i.e. size-exclusion chromatography). For example, gel filtration may be performed using a S200 sephadex size-exclusion column.

The different molecular species of the multimeric protein complexes are typically characterized by different molecular weights, such as low (pre-fusion stabilized trimeric spike glycoproteins), medium (post-fusion stabilized trimeric spike glycoproteins) and high (presumed oligomers of the trimeric post-fusion-stabilized spikes) molecular weight. Accordingly, in particular embodiments, the step of separating the pre-fusion stabilized trimeric spike glycoproteins complexes, the post-fusion stabilized trimeric spike glycoproteins and/or the presumed oligomers of the trimeric post-fusion-stabilized spikes, using gel filtration may comprise monitoring the separation of the different molecular species of the multimeric protein complexes by evaluating the separation of low, medium and high molecular weight fractions. The molecular weight fractions may be visualized by silver staining (typically after the SDS-PAGE).

A further aspect provides a trimeric protein complex or a composition of trimeric protein complexes obtainable by or obtained by the method as taught herein.

In particular embodiments, the composition comprises a trimeric protein complexes that assemble at a molecular weight of from 160 kDa to 210 kDa (corresponding to the pre-fusion state), and trimeric protein complexes that assemble at a molecular weight of from 250 kDa to 310 kDa (corresponding to the post-fusion state), preferably when being loaded on a SDS-PAGE 4-15% acrylamide gel.

Present inventors found that using a two-step purification (e.g. His-Trap & gel filtration) on trimeric protein complexes based on a CLR scaffold comprising one cysteine dramatically enriched in trimeric post-fusion stabilized spikes (representing approximately 75% and 25% pre-fusion form).

In particular embodiments, at least 50%, preferably at least 60%, even more preferably at least 70%, even more preferably at least 80%, such as at least 85%, at least 90%, at least 95%, such as at least 96%, at least 97%, at least 98%, at least 99% or 100%; of the trimeric protein complexes within the composition of trimeric protein complexes assemble at a molecular weight of from 250 kDa to 310 kDa (corresponding to the post-fusion state), preferably when being loaded on a SDS-PAGE 4-15% acrylamide gel.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and scope of the appended claims.

The herein disclosed aspects and embodiments of the invention are further supported by the following non-limiting examples.

### EXAMPLES

### Example 1

### Design of the Constructs

The 45-amino acids of the collagen-like region (CLR) of human ficolin-2, a normal human plasma molecule of the complement lectin pathway (LP), which is a natural scaffold for trimer formation, is used to produce multimeric spike version (FIG. 2). Importantly, the CLR contains a binding domain for the mannose-binding protein-associated serine protease 2 (MASP-2), a complement activator that cleaves C4 and C2. In that regard, the ficolin-2 CLR displays an intrinsic complement-mediated molecular adjuvant function.

Five different scaffolds from the ficolin-2 collagen-like region were designed (FIG. 3).

Scaffold 1 or “multimerization domain 1” (CLR1) consists of the original CLR from human ficolin 2 (as annotated under UniProt accession number Q15485.2). It displays a single cysteine in position 2 of the CLR from ficolin-2, This position is also being referred to in the present specification as “position 52” or “C52”, as it corresponds to the cysteine at position 52 of the amino acid sequence of the precursor form of human ficolin-2 as annotated under UniProt accession number Q15485.2 (i.e. including the native signal peptide). Expression of CLR1 generates monomers (approximately 220 kDa) and dimers (approximately 440 kDa) but no trimers (approximately 660 kDa) (FIG. 2B). CLR1 consists of 45 amino acids.

Scaffold 2 or “multimerization domain 2” (CLR2) consists of CLR1 that is C-terminally extended with the first 3 amino acids (“QPC”) of the fibrinogen-like region of human ficolin-2 (as annotated under UniProt accession number Q15485.2). The cysteine in the sequence “QPC” is also being referred to in the present specification as the cysteine at “position 98” or “C98”, as it corresponds to the cysteine at position 98 of the precursor amino acid sequence of human ficolin-2 as annotated under UniProt accession number Q15485.2. Scaffold 2 thus comprises two cysteines, namely cysteines C52 and C98, CLR2 generates monomers (approximately 220 kDa), dimers (approximately 440 kDa) and trimers (approximately 660 kDa) (FIG. 2B). CLR2 consists of 48 amino acids.

Scaffold 3 or “multimerization domain 3” (CLR3) consists of CLR1 that is N-terminally extended of the 25 amino acids of the N-terminal «<Cysteine-rich region» of human ficolin-2 (as annotated under UniProt accession number Q15485.2). The Cysteine-rich region of human ficolin-2 comprises a cysteine at position 32 (also corresponding to position 32 of the precursor amino acid sequence of human ficolin-2 as annotated under UniProt accession number Q15485.2), and is therefore also being referred to in the present specification as the cysteine at “position 32” or “C32”. Scaffold 3 thus displays 2 cysteines, the Cysteines C32 and C52, CLR3 generates a weak quantities of monomers and has been excluded from the scaffolds of interest.

Scaffold 4 or “multimerization domain 4” (CLR4) is the same as scaffold 1 (CLR1), with the exception of cysteine C52 being replaced by an Alanine (C52A). Scaffold 4 is a cysteine-free scaffold-. CLR4 generates monomers.

Scaffold 5 or “multimerization domain 5” (CLR5) Scaffold 5 consists of CLR1 that is C-terminally extended with the 10 first amino acids of the FLR of human ficolin-2 (as annotated under UniProt accession number Q15485.2) (QPCLTGPRTC (SEQ ID NO: 37). The first cysteine in the sequence QPCLTGPRTC (SEQ ID NO: 37) is also being referred to in the present specification as the cysteine at “position 98” or “C98”, as it corresponds to the cysteine at position 98 of the precursor amino acid sequence of human ficolin-2 as annotated under UniProt accession number Q15485.2. The second cysteine in the sequence QPCLTGPRTC (SEQ ID NO: 37) is also being referred to in the present specification as the cysteine at “position 105” or “C105”, as it corresponds to the cysteine at position 105 of the precursor amino acid sequence of human ficolin-2 as annotated under UniProt accession number Q15485.2. Scaffold 5 thus comprises three cysteines, namely cysteines C52, C98 and C105, CLR5 generates monomers (approximately 220 kDa), dimers (approximately 440 kDa) and trimers (approximately 660 kDa) (FIG. 2B), CLR5 consists of 55 amino acids, CLR5 generates monomers, dimers and trimers with a slightly more important ratio in trimers than the one obtained with CLR2.

[S]¹ and [S]² as referred to in example 2 can indifferently be obtained from the CLR1 or CLR2 constructs using gel filtration purification, while trimers can only come from CLR2 constructs.

The vector pEF-IRESpac was opened in Eco RI and Not I in the multiple cloning site (MCS) to introduce the synthetic fragment composed of 1) a signal sequence, 2) the N terminal domain of the SARS-CoV-2 Spike WT (Wuhan) protein, 3) the Receptor Binding Domain (RBD) of the Spike protein (aa 319-541 of SEQ ID NO2: 9), 4) the Heptad Repeat 1 domain of the Spike protein (aa 816-837 of SEQ ID NO: 9), 5) the Heptad Repeat 2 domain of the Spike protein (aa 835-588 of SEQ ID NO: 9), 6) the SGGGGS (SEQ ID NO: 1) linker, 7) the multimerization domain including the collagen-like region (CLR), 8) the poly-histidine tag (8 successive His) (FIG. 1 and FIG. 2B).

The amino acid and nucleic acid sequences of the Spike-CLR1 synthetic gene cloned in pEF-IRESpac expression vector are represented in FIG. 4.

As a control, Trastuzumab scFv (TRA scFv) was fused to CLR1 (monomeric and dimeric TRA scFv; about 40 k Da and 80 kDa respectively).

As further explained in Example 20, present inventors have found using cryo-EM that the “dimers” as referred to herein are in fact trimeric spike protein complexes in a post-fusion stabilized state; and the “monomers” as referred to herein are in fact trimeric spike protein complexes in a pre-fusion stabilized state.

### Example 2

### Formation of Dimeric SARS-CoV-2 Spike and Purification Thereof Using Immobilised Metal Chelate Chromatography (His-Trap Purification)

### Material & Method

### Transfection

Transfection: Cells were transfected in HEK293T cells with the pEF-IRESpac Spike CLR1 constructs in 6-well plate (1.3×10⁶ cells/well) for 48 h in optiMEM using lipofectamin 2000 (Thermo Fisher catalog No 11668019).
Subcloning: After 2 days, cells were then trypsinised and transferred into 10 cm cell culture dish with complete medium with selection antibiotic (5 μg/ml Puromycin). After about 2 weeks, colony-forming cell clones (CFC) that were resistant to the antibiotic selection appeared and started expanding. Individual colony-forming cell clones were individually manually picked-up and transferred into wells from a 96-well plate. After a week, individual cell clones expanded and the supernatants being consumed started turning yellowish.
Screening: This step allows quantifying the molecules produced into the supernatants of individual colonies (cell clones) in order to identify the best-expressing cell clones. This is the step of optimisation of the expression yields of soluble recombinant multimeric spikes, is important for scaling-up and large-scale molecule productions, Screening of supernatants from individually picked-up cell clones was performed using HEK293T/ACE2⁺/TMPRSS2⁺/mCherry⁺ cells (also referred to herein as HEK293T/ACE2 cells) (GeneCopocia™, SL222) commercial target cells, Superatants from of individual colony-forming cell clones were incubated with HEK293T/ACE2 cells (150,000 cells/well). Then cells were incubated with a rabbit anti-HIS antibody and a secondary antibody (goat anti-rabbit/AF 647). Cells were then analysed using flow cytometry.
Scaling-up of production of the soluble recombinant multimeric SARS-CoV-2 spikes: The best clones identified by FACS were expanded for further large scale productions. Cells were successively expanded in large culture flasks and then were cultured for 24 h in 5-chamber cell stacks in 500 ml complete DMEM medium. The next day, DMEM was replaced by serum-free optiMEM medium and cells were further cultured in optiMEM medium for 48 h corresponding to the first production. After 2 days, cells were boosted for 24 h with complete DMEM medium, and then a second 48 h cycle with optiMEM for a second production. Multimer-containing optiMEM (1 L) was filtered using Nalgene 1 L 0.22μ PVDF vaccum filter units.
His-Trap purification using multi-stepwise imidazole gradients: A 5 ml Excel Nickel His-Trap column was connected to a peristatic pump, and filtered optiMEM supematant was passed through the column for 5 days as a closed-loop (flow rate 2 ml/min) in order to immobilise the soluble dimeric recombinant SARS-CoV-2 spike glycoproteins to the column.

The column was then connected to a FPLC, and a stepwise imidazole gradient (50, 100, 150, 1000 mM imidazole) was applied to eluate the dimeric spikes (Data not shown). The eluates for each imidazole concentration were concentrated using centricon with 50 kDa molecular weight cut-off (MWCO). The 4 concentrated eluates were analysed using SDS-PAGE followed by a Western blotting onto PVDF membrane. The PVDF membrane was revealed using a rabbit anti-HIS pAb, followed by a goat anti-rabbit IgG AF647-conjugated. The membrane was scanned using a Thyphoon imager (data not shown).

For the His-Trap purification of the final batches of [S]², [S]¹ and control Trastuzumab scFv.CLR1 used for the vaccination of mice the number of stepwise imidazole gradients was increased (10, 20, 30, 40, 50, 60, 70, 80, 90, 100 and 1000 mM imidazole). The “low-valence” and “high-valence” fractions correspond to the pooled concentrated fractions from the 30-40 mM and 80-1000 mM imidazole steps, respectively.

### Results

To their surprise, present inventors found that the transfection of a vector encompassing the nucleic acid sequence encoding the Spike-CLR1 synthetic gene into HEK293T cells lead to the expression and formation of monomeric (about 220 kDa) and dimeric protein complexes (about 440 kDa), but no detectable level of trimeric protein complexes (about 660 kDa).

Subsequently, present inventors aimed at separating the Spike-CLR1 monomeric and dimeric protein complexes using His-Trap purification.

The chromatogram shows a first peak at 50 mM imidazole and a second peak at 1 M imidazole (data not shown). The western blot of the 4 concentrated elution fractions shows that the first lane corresponding to the first peak (50 mM imidazole) contains mainly a monomeric spike (˜220 kDa) and a degraded form (˜110 kDa). The upper band corresponds to the dimeric form (˜440 kDa). A cleaved form corresponds to the S2 domain of the spike fused to the CLR1.His8x, since it is recognised by the anti-HIS. This fragment is not recognised by an anti-SARS-CoV-2 S1-Spike antibody (data not shown). The second lane shows a major band corresponding to the monomeric spike, a upper band corresponding to the dimeric spike that is stronger than in lane 1, and a weak band of degraded spike. The third and fourth lanes show 2 bands of same intensity corresponding to the monomeric and dimeric spikes. The IMAC purification allows a relative separation (enrichment) of the dimeric spike that also contains the monomeric counterpart. The proportion of dimers, monomers and degraded forms were calculated using Image J and the calculation of the area under the curve function (data not shown), Dimers represent about 45% in fractions 3 and 4.

For the His-Trap purification of the final batches of “dimeric ([S]²)”, “monomeric ([S]¹)” and control Trastuzumab scFv.CLR1 used for the vaccination of mice, the pooled “low-valence” early elution fractions contain the monomeric spike as well as degraded form of the monomeric spike, but no dimeric spike (FIG. 5). In contrast, the pooled “high-valence” late elution fractions contain dimeric and monomeric spikes and no degraded spike (FIG. 5). The degraded forms are the S2 extracellular domain of the Spike followed by CLR1.His8x, detected using the anti-his pAb but not using a polyclonal rabbit anti-Spike whose immunogen was the S1 extracellular domain of the SARS-CoV-2 Spike (data not shown). The “low-valence” and “high-valence” batches were used for the vaccination of BALB/c mice.

It is noted that the “dimeric ([S]²)” or “[S]²” used for the vaccination of mice refers to a combination of monomeric and dimeric protein complexes, while the “monomeric ([S]¹)” or “[S]¹” used for the vaccination of mice refers to monomeric dimeric protein complexes.

As further explained in Example 20, present inventors have found using cryo-EM that the “dimers” as referred to in present example are in fact trimeric spike protein complexes in a post-fusion stabilized state; and the “monomers” as referred to in present example are in fact trimeric spike protein complexes in a pre-fusion stabilized state. Accordingly, the “dimeric ([S]²)” or “[S]²” used for the vaccination of mice are in fact a combination of pre-fusion trimeric spikes and post-fusion trimeric spikes, while the “monomeric ([S]¹)” or “[S]¹” used for the vaccination of mice are in fact pre-fusion trimeric spikes.

### Example 3

### Dose-Response Binding of Soluble Recombinant “Dimeric ([S]²)” Versus “Monomeric ([S]¹)” SARS-CoV-2 Spike Glycoproteins and to HEK293T/ACE2⁺/TMPRSS2⁺ or VeroE6 Cells

### Material & Methods

HEK293T/ACE2+/TMPRSS2+/mCherry+cells (also referred to herein as HEK293T/ACE2 cells) (GeneCopocia™, SL222), VeroE6 kidney cells from C. aethiops (ATCCφ CRL-1586™) and HEK293T (ATCC® CRL-3216™) control cells (150,000 cells/well) were incubated with two-fold serial dilutions of [S]¹ and [S]² (starting concentration was 50 μg/ml) for 30 min at 4° C. After washing, cells were stained with a rabbit anti-His pAb, followed by a goat anti-rabbit IgG AF647-conjugated pAb (30 min. at 4° C.). After washing, cells were fixed using 1% paraformaldehyde in PBS, and then cells were analysed using flow cytometry.

The soluble recombinant “dimeric ([S]²)” SARS-CoV-2 spike glycoproteins as used in present example correspond to “high-valence” sample from Example 2.

The soluble recombinant “monomeric ([S]¹)” SARS-CoV-2 spike glycoproteins as used in present example correspond to “low-valence” sample from Example 2.

### Results

Comparing the MFI anti-His signal between HEK293T/ACE2 (FIG. 6A) and control HEK293T (FIG. 6B), showed that the binding of [S]² and [S]¹ is specific to the presence of ACE2. On HEK293T/ACE2 and on VeroE6, at same concentration, the MFI anti-His signal for [S]²is up to 4.35-fold and 1.6-fold superior than for [S]¹, respectively (FIGS. 6A, C and D). The MFI anti-His signal for [S]² is 25.4-fold superior on HEK293T/ACE2 when compared to VeroE6 (FIGS. 6A and C). Thus, [S]¹ and [S]² specifically recognize ACE2, and due to its dimeric nature, [S]² displays a stronger anti-His signal than its monomeric counterpart on target cells at same concentration.

As further explained in Example 20, present inventors have found using cryo-EM that the “dimers” as referred to in present example are in fact trimeric spike protein complexes in a post-fusion stabilized state; and the “monomers” as referred to in present example are in fact trimeric spike protein complexes in a pre-fusion stabilized state. Accordingly, the “dimeric ([S]²)” or “[S]²” as referred to in present example are in fact a combination of pre-fusion trimeric spikes and post-fusion trimeric spikes, while the “monomeric ([S]¹)” or “[S]¹” as referred to in present example are in fact pre-fusion trimeric spikes.

### Example 4

### Comparative Study of the Ability of Soluble Recombinant SARS-CoV-2 “dimeric ([S]²)” Versus “Monomeric ([S]¹)” Spike Glycoproteins to Compete with the Binding of Anti-SARS-CoV-2 Spike Antibodies from COVID-19 Patient Serum to Coated SARS-CoV-2 Spike (S) or SARS-CoV-2-S-RBD Using Meso Scale Discovery (MSD) Multiplex Assay

### Material & Methods (FIG. 7A)

MSD 96-well 10-spot plates (MSD COVID-19 Panel 1, #K15362U; wells are coated with 10 different antigens from viral proteins/peptides including SARS-CoV-2 Spike and SARS-CoV-2 S1 RBD) were used according to the manufacturer's instructions. The incubation with serum from convalescent COVID-19 patient allows the binding of patient Abs to SARS-CoV-2 S and SARS-CoV-2 S-RBD. Serial dilution of [S]² versus [S]¹ and control TRA scFv.CLR1 (starting concentration was 100 μg/ml) were pre-incubated with the patients sera for 1 h at 4° C. prior to incubate to the MSD multi-spot 96-well plate. The plate was revealed with sulfo-tagged anti-human IgG (FIG. 7A) and read on MSD instrument QuickPlex SQ 120.

The soluble recombinant “dimeric ([S]²)” SARS-CoV-2 spike glycoproteins as used in present example correspond to “high-valence” sample from Example 2.

The soluble recombinant “monomeric ([S]¹)” SARS-CoV-2 spike glycoproteins as used in present example correspond to “low-valence” sample from Example 2.

### Results

The experiment showed that, at 100 μg/ml, [S]², but neither [S]¹ nor the control TRA scFv.CLR, led to 50% binding inhibition of anti-Spike antibodies to both SARS-CoV-2 S or SARS-CoV-2-S-RBD (FIG. 7B). This experiment indicates that the antibodies from CoviD patients recognise only the dimeric spike but not its monomeric counterpart, indicating that likely only soluble recombinant dimeric spike are able to elicit neutralising antibodies.

As further explained in Example 20, present inventors have found using cryo-EM that the “dimers” as referred to in present example are in fact trimeric spike protein complexes in a post-fusion stabilized state; and the “monomers” as referred to in present example are in fact trimeric spike protein complexes in a pre-fusion stabilized state. Accordingly, the “dimeric ([S]²)” or “[S]²” as referred to in present example are in fact a combination of pre-fusion trimeric spikes and post-fusion trimeric spikes, while the “monomeric ([S]¹)” or “[S]¹” as referred to in present example are in fact pre-fusion trimeric spikes.

### Example 5

### Activation of Human Complement by Recombinant Soluble SARS-CoV-2 spike “[S]¹” and “[S]²”

### Material & Methods (FIG. 8A)

HEK293T/ACE2 cells (150,000 cells per assay) were incubated with 10 μg/ml purified soluble recombinant [S]² and [S]¹ SASR-CoV-2 Spike glycoproteins (30 min. at 4° C.) (FIG. 8B) or with Trastu scFv.CLR1 or no molecule as negative controls. After washing with PBS/10% FBS, cells were incubated with 25% normal human serum (NHS) (decomplemented or not) from 2 different healthy donors in gelatin veronal buffer supplemented with Ca⁺⁺ and Mg⁺⁺ (GVB⁺⁺) for 30 min. at 37° C. After washing, cells were stained with a mouse anti-human C3/C3b/iC3b mAb (clone 10C7) and a goat anti-rabbit IgG AF647-conjugated pAb.

The soluble recombinant “dimeric ([S]²)” SARS-CoV-2 spike glycoproteins as used in present example correspond to “high-valence” sample from Example 2.

The soluble recombinant “monomeric ([S]¹)” SARS-CoV-2 spike glycoproteins as used in present example correspond to “low-valence” sample from Example 2.

### Results

[S]² and [S]¹ in a far lesser extent (MFI C3b 9000 versus 2500) elicit C3b deposition on HEK293T/ACE2 cells, whereas the MFI C3b for the control molecule was the same than in the absence of molecules (MFI C3b 1800) (FIG. 8C). When the NHS is decomplemented, the C3b deposition is completely abrogated, independently of the presence of [S]² or [S]¹.

s further explained in Example 20, present inventors have found using cryo-EM that the “dimers” as referred to in present example are in fact trimeric spike protein complexes in a post-fusion stabilized state; and the “monomers” as referred to in present example are in fact trimeric spike protein complexes in a pre-fusion stabilized state. Accordingly, the “dimeric ([S]²)” or “[S]²” as referred to in present example are in fact a combination of pre-fusion trimeric spikes and post-fusion trimeric spikes, while the “monomeric ([S]¹)” or “[S]¹” as referred to in present example are in fact pre-fusion trimeric spikes.

### Example 6

### Flow Cytometry Analysis of the Dimeric Spike-Mediated C3b Depositions on HEK293T/ACE2 Cells

### Material & Method

HEK293T/ACE2 cells (150 000 cells per assay) were incubated with 1 μg purified soluble recombinant dimeric SASR-CoV-2 spike glycoproteins [S]² (10 μg/ml, 30 min. at 4° C.) or with no molecule as negative control. The soluble recombinant “dimeric ([S]²)” SARS-CoV-2 spike glycoproteins as used in present example correspond to “high-valence” sample from Example 2.

After washing with PBS/10% FBS, cells were incubated with various percentages of normal human serum (NHS) (10, 15, 20, 25%) in gelatin veronal buffer supplemented with Ca⁺⁺ and Mg⁺⁺ (GVB⁺⁺) for 30 min. at 37° C. (FIG. 9A-B). HEK293T/ACE2 cells (150,000 cells per assay) were incubated with a serial dilution of purified [S]² (starting at 20 μg/100 μl). Cells were then incubated with 25% NHS/GVB⁺⁺ for 30 min. at 37° C. (FIG. 9C-D). After washing, cells were stained with a mouse anti-human C3/C3b/iC3b mAb (10C7) (FIG. 9A-C) and a rabbit anti-human C3d pAb (FIG. 9 B-D), and a goat anti-mouse IgG AF488-conjugated pAb and a goat anti-rabbit IgG AF647-conjugated pAb. Cells were then washed, fixed with PBS/1% PFA and analysed using flow cytometry.

### Results

Present inventors observed a C3b deposition that was serum (FIG. 9A-B) or molecule (FIG. 9 C-D) dose-dependent. The C3b/C3d deposition patterns were similar (with the exception of the MFI levels) when using the anti-C3b or anti-C3d Abs (FIG. 9A, B). This experiment confirms that the soluble recombinant dimeric SARS-CoV-2 spike glycoprotein elicits complement activation that leads to C3b depositions on HEK293/ACE2 target cells.

As further explained in Example 20, present inventors have found using cryo-EM that the “dimers” as referred to in present example are in fact trimeric spike protein complexes in a post-fusion stabilized state; and the “monomers” as referred to in present example are in fact trimeric spike protein complexes in a pre-fusion stabilized state. Accordingly, the “dimeric ([S]²)” or “[S]²” as referred to in present example are in fact a combination of pre-fusion trimeric spikes and post-fusion trimeric spikes, while the “monomeric ([S]¹)” or “[S]¹” as referred to in present example are in fact pre-fusion trimeric spikes.

### Example 7

### Comparative Study of “[S]²” Versus “[S]¹”-Mediated Complement Activation on HEK293T/ACE2 Cells Using Several Serum Conditions

### Material & Methods

See materials and methods of Example 5. In this experiment, 50 μg/ml purified [S]² and [S]¹ (5 μg in 100 μl PBS/10% FBS/150,000 HEK293T/ACE2 cells/well) were used.

### Results

As previously observed in FIG. 8C, [S]² elicit greater complement activation (x3,7 for C3b, x2.75 for C3d) than [S]¹. The greatest signal for all 4 antibodies was observed for the [S]² together with 25% NHS (GVB⁺⁺) serum condition. Inhibition of the classical/lectin complement pathways (GVB⁺) totally abrogates all signals (C3b, C3d, C4d, C5b9). Inhibition of the classical complement pathway (C1q-deficient HS) diminishes the C3b/C3d (of factor 2.75), C4d and in a lesser extend C5b9 (of factor 1.5) signals. Inhibiting the alternative complement pathway (FB-deficient HS) diminishes the C3b/C3d (of factor 3.4), has no effect on C4d deposition and diminishes the C5b9 signal (of factor 1.5). Inhibition of the complement terminal pathway (C5-deficient HS) only abrogates the C5b9 formation (completely) (FIG. 10A-D).

Altogether, both lectin/classical pathways and alternative pathways are involved in the [S]²-mediated complement activation on HEK293/ACE2 cells, the former having a more important role then the latter.

As further explained in Example 20, present inventors have found using cryo-EM that the “dimers” as referred to in present example are in fact trimeric spike protein complexes in a post-fusion stabilized state; and the “monomers” as referred to in present example are in fact trimeric spike protein complexes in a pre-fusion stabilized state. Accordingly, the “dimeric ([S]²)” or “[S]²” as referred to in present example are in fact a combination of pre-fusion trimeric spikes and post-fusion trimeric spikes, while the “monomeric ([S]¹)” or “[S]¹” as referred to in present example are in fact pre-fusion trimeric spikes,

### Example 8

### Vaccination of BALB/c Mice

### Material & Methods (FIG. 11)

Eight-week-old female BALB/c OlaHsd mice were obtained from Envigo (Horst, Netherlands) and kept in a specific pathogen free animal facility with unlimited access to food and water. Animal handling procedures met the European guidelines and were approved by the National Animal Research Authority (project DII-2020-03). At days 0 and 14, groups of 10 mice were injected intraperitoneally with 200 μl of the different vaccination solutions described in the table of FIG. 11. The SARS-CoV-2 spike used in groups 5-12 shown in FIG. 11 is the SARS-CoV-2 spike of the SARS-CoV-2 Wuhan variant. Blood was collected (lateral caudal vein) before the first injection (day −7), between both injections (day 14) and at sacrifice (day 28, cardiac puncture). The serum was used to test Ig profile and neutralization efficacy of the antibodies.

The soluble recombinant “dimeric ([S]²)” SARS-CoV-2 spike glycoproteins as used in present example are from the pooled “high valence” as referred to in Example 2, The soluble recombinant “monomeric ([S]¹)” SARS-CoV-2 spike glycoproteins as used in present example are from the pooled “low valence” as referred to in Example 2.

IgG1 serology in vaccinated mice: MSD 96-well 10-spot plates (MSD COVID-19 Panel 1, #K15362U) were used according to the manufacturer's instructions. After incubation with diluted mouse serum (1/100) and washes, the sulfo-tagged anti-mouse IgG1 detection antibody (from MSD kit Mouse Isotyping Panel 1, #K15183B-1) was used according to manufacturer's instructions to measure IgG1 specific signal. The plates were read on MSD instrument Quick Plex SQ 120.

IgG2a serology in vaccinated mice: MSD 96-well 10-spot plates (MSD COVID-19 Panel 1, #K15362U) were used according to the manufacturer's instructions. After incubation with diluted mouse serum (1/100) and washes, the sulfo-tagged anti-mouse IgG2a detection antibody (from MSD kit Mouse Isotyping Panel 1, #K15183B-1) was used according to manufacturer's instructions to measure IgG2a specific signal. The plates were read on MSD instrument Quick Plex SQ 120.

IgG2a serology in vaccinated mice: MSD 96-well 10-spot plates (MSD COVID-19 Panel 1, #K15362U) were used according to the manufacturer's instructions. After incubation with diluted mouse serum (1/100) and washes, the sulfo-tagged anti-mouse IgG2b detection antibody (from MSD kit Mouse Isotyping Panel 1, #K15183B-1) was used according to manufacturer's instructions to measure IgG2b specific signal. The plates were read on MSD instrument QuickPlex SQ 120.

IgG3 serology in vaccinated mice: MSD 96-well 10-spot plates (MSD COVID-19 Panel 1, #K15362U) were used according to the manufacturer's instructions. After incubation with diluted mouse serum (1/100) and washes, the sulfo-tagged anti-mouse IgG3 detection antibody (from MSD kit Mouse Isotyping Panel 1, #K15183B-1) was used according to manufacturer's instructions to measure IgG3 specific signal. The plates were read on MSD instrument Quick Plex SQ 120.

Anti-SARS-CoV-2 Spike (S)-specific IgG1, IgG2a, IgG2b and IgG3: Same as for IgG1, IgG2a, IgG2b, and IgG3 above, MSD 96-well 10-spot plates (MSD COVID-19 Panel 1, #K15362U) were used according to the manufacturer's instructions. After incubation with diluted mouse serum (1/100) and washes, the sulfo-tagged anti-mouse IgG1, or IgG2a or IgG2b or IgG3 detection antibodies (from MSD kit Mouse Isotyping Panel 1, #K15183B-1) were used according to manufacturer's instructions to measure the specific signal due to the different isotypes. The plates were read on MSD instrument Quick Plex SQ 120.

Anti-SARS-CoV-2 Spike receptor binding domain (S-RBD))-specific IgG1, IgG2a, IgG2b and IgG3: Same as for IgG1, IgG2a, IgG2b, and IgG3 above, MSD 96-well 10-spot plates (MSD COVID-19 Panel 1, #K15362U) were used according to the manufacturer's instructions. After incubation with diluted mouse serum (1/100) and washes, the sulfo-tagged anti-mouse IgG1, or IgG2a or IgG2b or IgG3 detection antibodies (from MSD kit Mouse Isotyping Panel 1, #K15183B-1) were used according to manufacturer's instructions to measure the specific signal due to the different isotypes. The plates were read on MSD instrument Quick Plex SQ 120.

### Results

### IgG1 Serology in Vaccinated Mice (FIG. 12)

The results show that the use of the 10 μg dose of recombinant [S]² together with 1 μg CpG induced the stronger IgG1synthesis in vaccinated mice. Indeed, a clear and significant increase in IgG1against SARS-CoV-2 S when 10 μg dimeric recombinant spike [S]² is used for the vaccination, in the absence of CpG (group 11) or presence of 1 μg CpG adjuvant (group 12) added to the vaccination solution (group 11 compared to the others: ****p to groups 2, 5, 6, 7, 8; ***p to groups 3 and 4; *p to group 9, Group 12 compared to the others: ****p to groups 2, 3, 4, 5, 6, 7, 8; ***p to group 9; **p to groups 1 and 10). Mice vaccinated with monomeric [S]¹ solutions displayed an increase in specific IgG1signal towards [S]¹, although not significant. In the vaccination with recombinant spike [S]² with CpG (group 12), the increase of IgG1specific to SARS-CoV-2RBD was also significant (****p to groups 2, 5, 6, 7; ***p to group 4; **p to groups 3 and 8). In the vaccination with recombinant [S]² without CpG (group 11), the increase of

IgG1specific to SARS-CoV-2NTD was also significant (**p to groups 2, 4, 5, 6, 7, 8, 9; *p to group 10), as well as in the vaccination with [S]² and CpG (group 12) (**p to groups 2, 5, 6, 7, 8; *p to groups 3, 4 and 9). The synthesis of IgG1specific for SARS-CoV-2 Nucleocapsid was not significant in none of the vaccinated groups compared to the others, One way ANOVA statistical analysis: *p<0.05; **p<0.01; ***p<0.001; ****p<0.0001. The SARS-CoV-2 Nucleocapsid is characterized by aa 48-174 of a protein sequence as annotated under Uniprot (www.uniprot.org) accession number P0DTC9.1.

### IgG2a Serology in Vaccinated Mice (FIG. 13)

The results show that the use of the 10 μg dose of recombinant [S]² together with 1 μg CpG induced the stronger IgG2a synthesis in vaccinated mice. Indeed, a very significant increase in IgG2a against SARS-CoV-2 S protein was observed when 10 μg dimeric recombinant spike [S]² was used for the vaccination in presence of 1 μg CpG adjuvant added to the vaccination solution (****p to all the other groups). The same was not observed with the monomeric [S]¹. In the same vaccination conditions [S]²+CpG, the increase of IgG2a specific to SARS-CoV-2RBD domain was also highly significant (****p to all the groups except naïve mice) as well as the increase of IgG2a specific to S N-terminal domain (****p to groups 2, 4, 5, 6, 7, 8, 9 and 10; ***p to group 11). The synthesis of IgG2a specific for SARS-CoV-2 Nucleocapsid was not changing significantly. One way ANOVA statistical analysis: ****p<0.0001, ***p<0.001.

### IgG2b Serology in Vaccinated Mice (FIG. 14)

The results show a very significant increase in IgG2b against SARS-CoV-2 S protein when 10 μg dimeric recombinant spike [S]² was used for the vaccination in presence of 1 μg CpG adjuvant added to the vaccination solution (****p to all the groups except **p to group 1 and ***p to group 11). The same was not observed with the monomeric [S]¹. In the same vaccination conditions, the increase of IgG2b specific to SARS-CoV-2RBD domain was also significant (*p to groups 2, 3, 5, 6, 7,8 and 10) as well as the increase of IgG2b specific to S N-terminal domain (***p to 2, 4, 5, 6, 7, 8, 9, 11; **p to group 3 and *p to group 10). The synthesis of IgG2a specific for SARS-CoV-2 Nucleocapsid was not significant. One way ANOVA statistical analysis: *p<0.05; **p<0.01; ***p<0.001; ****p<0.0001.

### IgG3 Serology in Vaccinated Mice (FIG. 15)

The results show a very significant increase in IgG3 against SARS-CoV-2 S protein when 10 μg dimeric recombinant spike [S]² was used for the vaccination in presence of 1 μg CpG adjuvant added to the vaccination solution (****p to all the groups except not significant to group 1 and ***p to groups 3 and 11). The same was not observed with the monomeric [S]¹. In the same vaccination conditions, the increase of IgG3 specific to SARS-CoV-2RBD domain was also significant (***p to groups 2, 5, 6, 7, 8 and 9; **p to groups 4 and 10 and *p to groups 3 and 11) as well as the increase of IgG3 specific to S N-terminal domain (**p to groups 2, 4, 5, 6, 7, 8, 9, 10, 11; *p to group 3). The synthesis of IgG3 specific for SARS-CoV-2 Nucleocapsid was significant for the group 11 when compared to the other groups (****p to groups 5, 6 and 9; ***p to groups 4, 7 and 8; **p to groups 3 and 10), One way ANOVA statistical analysis: *p<0.05; **p<0.01; ***p<0.001; ****p<0.0001.

### Anti-SARS-CoV-2 Spike (S)-Specific IgG1, IgG2a, IgG2b and IgG3 (FIG. 16)

Vaccinations with [S]¹ and [S]² induced humoral responses towards CoV-2-S, with IgG1being the most represented Ab isotype without CpG adjuvant. The use of dimeric Spike [S]² induced a stronger general antibody response than [S]¹. The presence of CpG adjuvant in the vaccination solution supported the increase of Ab titres, most pronounced for IgG2a, Ab responses were dose-dependent, whereby a 10-times higher [S]² concentration in the vaccination solution (10 μg injected [S]², second part of FIG. 16) induced more than 2 log increase in the IgG response compared to the1 μg injected [S]² (first part of FIG. 16). Control injections with CpG adjuvant only or with Trastu scFv-CLR induced no anti-SARS-CoV S Abs. RLU: Read Log Update.

### Anti-SARS-CoV-2 Spike Receptor Binding Domain (S-RBD)-Specific IgG1, IgG2a, IgG2b and IgG3 (FIG. 17)

Vaccinations with [S]¹ and [S]² induced humoral responses towards CoV-2-S-RBD, with IgG1 being the most represented Ab isotype without CpG adjuvant. The use of dimeric Spike [S]² induces a stronger general antibody response than with [S]¹. The presence of CpG adjuvant in the vaccination solution supports the increase of AB titres, most pronounced for IgG2a. Ab responses were dose-dependent, whereby a 10-times higher [S]² concentration in the vaccination solution (10 μg injected [S]², second part of FIG. 17) induced more than 2 log increase in the IgG response compared to the 1 μg injected [S]² (first part of FIG. 17). Control injections with CpG adjuvant only or with Trastu scFv-CLR induced no anti-SARS-CoV S Abs, RLU: Read Log Update.

As further explained in Example 20, present inventors have found using cryo-EM that the “dimers” as referred to in present example are in fact trimeric spike protein complexes in a post-fusion stabilized state; and the “monomers” as referred to in present example are in fact trimeric spike protein complexes in a pre-fusion stabilized state. Accordingly, the “dimeric ([S]²)” or “[S]²” as referred to in present example are in fact a combination of pre-fusion trimeric spikes and post-fusion trimeric spikes, while the “monomeric ([S]¹)” or “[S]¹” as referred to in present example are in fact pre-fusion trimeric spikes

### Example 9

### Surrogate Neutralization Assay

### Material & Methods

MSD 96-well 3-spot plates (MSD SARS-CoV-2 Panel 2, #K15386U) were used according to the manufacturer's instructions. After incubation 1 h with 25 μl diluted mouse serum or diluted sera from convalescent patients (dilutions 1/5, 1/25 and 1/50), or the calibrator solutions, the Sulfo-tag ACE2 solution was added to the wells for another 1 h incubation. After the washes, the MSD Gold reading buffer was added to the wells and the plates were read on MSD instrument QuickPlex SQ 120. Patient sera with neutralizing activity as well as control sera were derived from ongoing clinical studies on COVID-19 at the Luxembourg Institute of Health (Predi-COVID) (Fagherazzi et al., Protocol for a prospective, longitudinal cohort of people with COVID-19 and their household members to study factors associate with disease severity: the Predi-COVID study, BMJ Open, 2020: 10(11): e041834. Severity of COVID-19 was done according to the guidelines published by the National Institutes of Health (NIH) of the United States (https://www.covid19treatmentguidelines.nih.gov/overview/clinical-spectrum/).

The soluble recombinant “dimeric ([S]²)” SARS-CoV-2 spike glycoproteins as used in present example are from the pooled “high valence” as referred to in Example 2. The soluble recombinant “monomeric ([S]¹)” SARS-CoV-2 spike glycoproteins as used in present example are from the pooled “low valence” as referred to in Example 2.

### Results

The results indicate that mice vaccinated with [S]² synthesised antibodies are able to compete with ACE2 for binding to S or S-RBD. The mice injected with the highest-dose of [S]² (10 μg) and with CpG as adjuvant display the strongest inhibition (dark grey vertical bar), with equivalent effects as those from COVID-19 recovered patients (FIG. 18).

As further explained in Example 20, present inventors have found using cryo-EM that the “dimers” as referred to in present example are in fact trimeric spike protein complexes in a post-fusion stabilized state; and the “monomers” as referred to in present example are in fact trimeric spike protein complexes in a pre-fusion stabilized state. Accordingly, the “dimeric ([S]¹)” or “[S]²” as referred to in present example are in fact a combination of pre-fusion trimeric spikes and post-fusion trimeric spikes, while the “monomeric ([S]¹)” or “[S]¹” as referred to in present example are in fact pre-fusion trimeric spikes.

### Example 10

### Correlation of Serologic Antibody Levels and Surrogate Binding Inhibition of huACE2 to SARS-CoV-2 S and S-RBD Induced by Vaccination with Spike “[S]¹” and “[S]²”,

### Material & Methods

See Example 8 for IgG1 serology and Example 9 for surrogate neutralization.

### Results

The upper panel of FIG. 19A indicates that the highest competition for ACE2 binding to Spike (larger and darker part of the triangle on X axis) is obtained with the highest levels of Spike-specific IgG1(larger and darker part of the triangle on Y axis) in the mice injected with dimeric form of the recombinant Spike protein (symbols on the graph: large triangles) and more specifically when CpG is added to the vaccination solution (dark grey triangle, doted grey curve). The lower panel FIG. 19A shows the same tendency for the IgG1specific for the S-RBD domain. The upper and lower panels of FIG. 19 B illustrate a congruent result for the specific-IgG2a antibody response against Spike protein and S-RBD domain.

As further explained in Example 20, present inventors have found using cryo-EM that the “dimers” as referred to in present example are in fact trimeric spike protein complexes in a post-fusion stabilized state; and the “monomers” as referred to in present example are in fact trimeric spike protein complexes in a pre-fusion stabilized state. Accordingly, the “dimeric ([S]²)” or “[S]²” as referred to in present example are in fact a combination of pre-fusion trimeric spikes and post-fusion trimeric spikes, while the “monomeric ([S]¹)” or “[S]¹” as referred to in present example are in fact pre-fusion trimeric spikes.

### Example 11

### Flow Cytometry Analysis of the Binding of 4 Soluble Recombinant Trimeric SARS-CoV-2 Spike Glycoproteins

### Material & Method

pEF-IREPpac expression vectors bearing the cassettes encoding (i) the «All mutations in the Spike protein from B1.1.7 (UK), B1.351 (South-African) and P.1 (Brazilian) variants».CLR2, (ii) the 5 mutation Spike (L18F, K417N, E484K, N501Y and D614G).CLR2, (iii) the B1.351 Spike (South-African).CLR2 and (iv) the Original Wuhan Spike.CLR1 were transfected into HEK293T cells in optiMEM. After 48h hours, the supematants were collected and concentrated about 10× using Centricon.

The «All mutations in the Spike protein from B1.1.7 (UK), B1.351 (South-African) and P.1 (Brazilian) variants».CLR2 comprises the following mutations: del 69-70HV, del 144 Y, del242-244, L18F, T20N, P26S, D80A, D138Y, R190S, R246I, K417N, E484K, N501Y, A570D, D614G, H655Y, P681H, A701V, T761I, S982A, T1027I, and D1118H, The B1.351 Spike (South-African).CLR2 comprises del242-244, L18F, E484K, D80A, N501Y, R246I, D614G, K417N and A701V. The person skilled in the art will understand that, for example, del 144 Y refers to the deletion of the Y at position 70 of the Spike protein, and that, for example, N501Y designates a replacement of the N residue at position 501 of the Spike protein by a Y residue. The mutations are mutations compared to the sequence of the Spike protein of the ancestor SARS-CoV-2 isolate Wuhan-Hu-1.

HEK293T/ACE2 cells (150,000 cells/well) were incubated with Spike-containing 10-fold concentrated crude optiMEM supernatants from transient transfections for 30 min. at 4° C. After washing, cells were incubated with either a rabbit anti-HIS pAb or a rabbit anti-SARS-CoV-2 S1 Spike subunit mAb (Sino Biological #40150-R007), followed by a staining with a goat anti-rabbit IgG AF647-conjugated pAb. Cells were analysed using flow cytometry.

The Spike variants described in present example were purified using two-step purification, such as described in Example 19.

### Results

The MFI anti-His (FIG. 20A) or anti-spike (FIG. 20 B) indicate that the three constructs carrying the mutations fused to CLR2 are expressed and functional, and able to bind ACE2. The «All spike mutations from B1.1.7 (UK), B1.351 (South-African) and P.1 (Brazilian) variants».CLR2 and B1.351 Spike (South-African).CLR2 are the best expressed, then comes the 5 mutation Spike (L18F, K417N, E484K, N501Y and D614G). CLR2 and finally the lowest expression was observed with the first generation Spike.CLR1 as seen in FIG. 20A.

### Example 12

### Size-Exclusion (Gel Filtration) Chromatography (SEC) Using a Superdex™ 200 Increase Column (10/300 GL) for High-Resolution Gel Filtration in Small-Scale (mg) Preparative Purification

### Material & Method

Pre-purification using His-Trap Nickel column: Filtered optiMEM was loaded to a 5 ml His-Trap Excel column for 5 days using a peristaltic pump (flow-rate 2-3 ml/min) as a closed-loop. The His-Trap Excel 5 ml column previously loaded with the optiMEM supernatant was then connected to a Bio-Rad NGC Discover 10 FPLC system.
After an extensive washing with 20 mM imidazole (20 mM phosphate buffer pH7.2, 500 mM NaCl, 20 mM Imidazole). The 1M imidazole His-Trap fraction of purified spike was concentrated using 50 kDa MWCO Centricon to 250 μl final volume.
Purification using size-exclusion chromatography: The concentrated pre-purified Spikes were purified using a Bio-Rad NGC FPLC. The sample was injected to a 250 μl single-injection loading loop, then loading onto a gel filtration chromatography Superdex™ Increase S200 (10/300GL) prepacked column, and run at flow rate 0.1 ml/min. The running buffer is composed of a 20 mM phosphate buffer with 500 mM NaCl, pH7.2.

The chromatogram obtained upon purification shows that a first peak appeared at 8 ml elution volume and a second peak appeared at 10 ml elution volume (data not shown). Fractions of 500 μl were collected. Peaks 1 and 2 correspond to collected fractions 3-8 and 9-12, respectively. Collected fractions (2 to 14) were analysed using SDS-PAGE (non-reducing conditions) followed by a Western blotting (WB) analysis under non-reducing conditions. The WB was revealed with a rabbit anti-HIS pAb and a secondary goat anti-rabbit IgG AF647 conjugated pAb, Collected fractions were then analysed using flow cytometry. Five μl of each fraction were incubated with 150,000 HEK293/ACE2, Cells were then stained using a rabbit anti-His pAb and a goat anti-rabbit IgG pAb AF647-conjugated, Cells were analysed using flow cytometry (data not shown).

### Results

The first peak contains mainly the dimeric spike (about 70% of dimers) (data not shown). The second peak contains some dimers but mainly the monomeric forms (about 65 to 95%) and degraded forms of the soluble recombinant SARS-CoV-2 glycoproteins. The gel filtration thus clearly allowed separating the dimers (estimated size 440 kDa) from the monomers (estimated size 220 kDa) and degradation products (estimated size 120 kDa and 80 kDa, data not shown). In addition, the gel filtration allows at the same time to get rid of the imidazole, with no further need to perform a dialysis afterward. The strongest anti-HIS signal on HEK293T/ACE2 loaded with the different elution fractions and analysed by FACS was observed with the fractions 6 to 9 which correspond to the 2 peaks (data not shown).

As further explained in Example 20, present inventors have found using cryo-EM that the “dimers” as referred to in present example are in fact trimeric spike protein complexes in a post-fusion stabilized state; and the “monomers” as referred to in present example are in fact trimeric spike protein complexes in a pre-fusion stabilized state.

### Example 13

### Semi-Quantitative Flow Cytometry Analysis—Using HEK293/ACE2 Target Cells—of the Screening of the Supernatants from Individual Cell Clones Expressing Various Soluble Recombinant Trimeric SARS-CoV-2 Spike Glycoproteins

### Material & Method

The 4 transient transfections from FIG. 20 were stabilised using complete DMEM medium supplemented with 5 μg/ml Puromycin, Higher concentrations are lethal for the cells. After about 2 weeks, Puromycin-resistant cell clones appear in the dish. Using manual pick-up, individual clones are collected and transferred into a cell culture 96-well plate. For each construct, 95 individual clones were isolated. After a week, when the supernatants start turning yellowish, what indicates that the clones are well developed, supematants were screened, HEK293T/ACE2 cells (150,000 cells/well) were incubated with 170 μl Spike-expressing crude supernatants for 30 min. at 4° C. Supernatants from the bulks from which individual cell clones were picked-up were used as reference. Cells were washed with PBS/10% FBS, then were incubated with a rabbit anti-HIS pAb for 30 min. at 4° C., and with a goat anti-rabbit IgG AF647-conjugated pAb. Cells were fixed with PBS/1% paraformaldehyde, and analysed using flow cytometry.

### Results

The expression of spikes in the CLR scaffold CLR2 with 2 cysteines (C52 and C98) allowed getting clones with very high HIS signal, that were higher when compared to the expression of spikes in the CLR1 (data not shown). One very good clone was obtained for each of the three spikes “South-African” (SA) (clone D8, HIS MFI 7575), “all mutations from the 3 UK, Brazilian and South-African variants” (ALL) (clone B8, HIS MFI 12651) and 5-mutation spike (5M) (clones A4, B9, HIS MFI 6779 and 5167, respectively). For the primary construct with the Wuhan Spike.CLR1, the best clone (clone C8) displayed a 2904 HIS MFI, substantially lower when compared to the 3 Spikes expressed with the CLR2 (data not shown).

Present inventors selected 3 to 5 good clones for each construct and expanded and retested these. The best-expressing clones are cultured using Puromycin 20 mg/ml to increase further the expression yields. The screening for the best-expressing clones allows to benefit the highest expression of trimeric spikes.

### Example 14

### Western Blotting Analysis of the SA.CLR2, ALL.CLR2, 5M.CLR2, S.CLR1 from the Best-Selected Clones and Silver Staining. Western Blotting Analysis of Constructs SA.CLR4 and SA.CLR1 from Transient Transfection

### Material & Method

Four clones SA.CLR2 (B8, C12, D3, D8), 4 clones ALL.CLR2 (A9, B8, B12, C11), 3 clones 5M.CLR2 (A4, A11, B9) and 2 clones S.CLR1 (C8, H2) resulting from the screening described in Example 13 were expanded. Soluble recombinant Spike-containing complete DMEM supernatants (5 ml) were collected and incubated overnight at 4° C. with 50 μl agarose Ni beads to purify the Spike glycoproteins. Beads were washed twice with PBS and incubated with 1 M imidazole-containing elution buffer (20 mM phosphate buffer pH7.2, 500 mM NaCl, 1M Imidazole).

In addition, SA.CLR4 and SA.CLR1 constructs were transfected into HEK293T cells (as described in example 2). After 2 days, optiMEM was concentrated for SDS-PAGE and WB analysis.

Eluates from micro-purifications as well as concentrated optiMEM supernatants were analysed using SDS-PAGE followed by a Western blotting and the PVDF membrane was incubated with a rabbit anti-HIS pAb and a goat anti-rabbit IgG AF647-conjugated pAb. The PVDF membrane was revealed as described for Example 2 using a Typhoon imager. A “HiMark™ pre-stained protein standard” was used, the upper band of the protein ladder displaying at 460 MW which is close to MW of the soluble recombinant dimeric SARS-CoV-2 spike glycoprotein (data not shown).

### Results

The molecular pattern of the molecular species observed in WB clearly indicate that (data not shown):
CLR1 (C52) generates mostly dimeric (apparent MW 440 kDa) and monomeric (apparent MW 220 kDa) spikes and only some residual trimeric spikes (apparent MW 660 kDa),
CLR2 (C52, C98) generates monomeric, dimeric (apparent MW 440 kDa) and trimeric spikes (apparent MW 660 kDa),
CLR4 (no cysteine) generates monomeric spikes (apparent MW 220 kDa) and only some residual dimers (apparent MW 440 kDa),

Present inventors concluded that the cysteine environment around the CLR plays a crucial role in the production and stabilisation of dimers and/or trimers. More particularly, the presence or absence of C52 and C98 in the multimerization domain appear to control the molecular species formed, CLR4 lacking both cysteines C52 and C98 brings additional evidence, the absence of both cysteines in CLR4 leading to the sole generation of monomers.

As further explained in Example 20, present inventors have found using cryo-EM that the “dimers” as referred to in present example are in fact trimeric spike protein complexes in a post-fusion stabilized state; and the “monomers” as referred to in present example are in fact trimeric spike protein complexes in a pre-fusion stabilized state.

### Example 15

### Silver Staining Analysis Following SDS-PAGE of the Fractions 5M.CLR2

### Material & Method

5M.CLR2 (best clone B9, see Example 13) was produced in a cell stack pre-purified using His-Trap and purified using gel filtration as described in example 2. Collected fractions from the gel filtration purification were analysed using SDS-PAGE under non-reducing conditions, followed by a silver staining (data not shown).

### Results

As previously described, 3 molecular species co-exist. The fractions 12-16 (first peak) of the gel filtration purification concentrate the trimers which remains the main molecular species, despite the presence of dimers and monomers. In fractions 12-13, the trimers represent more than 50% of the whole, From fraction 17 (second peak), only monomers are eluted, representing more than 70% of the whole molecular species. Degradation products are well separated and are found in third peak (fraction 27). The gel filtration (i) dramatically enriched in trimeric molecular species, while (ii) removing the large majority of monomers and all degraded forms. These data bring evidence that trimeric spikes are produced when CLR2 (displaying 2 cysteines) is used. The gel filtration allows removing 71% of monomeric spikes (when comparing the amount of monomers in fractions 12-16 and 17-20). The His-Trap pre-purified product (that was further purified using gel filtration) was loaded on gel, displaying from the top trimers (660 kDa), dimers (440 kDa), monomers (220 kDa) and 2 degraded forms (130 and 90 kDa) (data not shown).

As further explained in Example 20, present inventors have found using cryo-EM that the “dimers” as referred to in present example are in fact trimeric spike protein complexes in a post-fusion stabilized state; and the “monomers” as referred to in present example are in fact trimeric spike protein complexes in a pre-fusion stabilized state.

### Example 16

### Western Blotting Analysis of the SA.CLR4

Only monomeric spikes and some residual dimers (less than 1%) are produced when CLR4 (displaying no cysteines) is used (data not shown).

As further explained in Example 20, present inventors have found using cryo-EM that the “dimers” as referred to in present example are in fact trimeric spike protein complexes in a post-fusion stabilized state; and the “monomers” as referred to in present example are in fact trimeric spike protein complexes in a pre-fusion stabilized state.

### Example 17

### Vaccination of BALB/c Mice

### Material & Methods

Present inventors used the Beta spike variant-displaying triple RBD substitution K417N, E484K, N501Y, which is associated with increased infectivity and lethality-as soluble spike protein immunogen. B.1.351 was reported to be resistant to neutralization by most NTD mAbs and several mAbs to receptor-binding motif (RBM) on RBD, mainly because of the E484K substitution. B.1.351 is more resistant to neutralization by convalescent plasma and vaccine sera, respectively.

Vaccination of BALB c with Dimeric or Trimeric spikes: A second round of vaccination in BALB/c mice was performed according the same immunization scheme as used in FIG. 11, including 2 successive intraperitoneal injections of 200 μl, at 2 weeks interval (D0 and D14). In this second round, nine groups of mice were injected using the vaccination solutions depicted in the following table: 1. No injection (naïve mice, n=5); 2. 500 μg Alum+2 μg CpG (n=5); 3. 10 μg Dimeric Beta spike (expressed with CLR1 scaffold) alone (n=5); 4. 10 μg Dimeric Beta spike+CpG (n=10); 5. 10 μg Dimeric Beta spike+Alum (n=10); 6. 10 μg Dimeric Beta spike+Alum and CpG (n=10); 7. 10 μg Trimeric Beta spike (expressed with CLR2 scaffold)+CpG (n=10); 8. 10 μg Trimeric Beta spike+Alum (n=10); 9. 10 μg Trimeric Beta spike+Alum and CpG (n=10).

It is noted that, with exception of the origin of the SARS-CoV-2 Spike (Wuhan vs Beta variant), no further differences are present between the structure of the dimeric spike of present example compared to the dimeric spike of Example 8 (FIG. 11).

The “Dimeric spike” was purified using a two-step purification process, such as described in example 19. Accordingly, reference to “Dimeric spike” (when used in combination with CLR), “Dimeric spike [S2]” or “[S2]” in present example, corresponds to a mix comprising monomeric as well as dimeric protein complexes, Reference to “monomeric spike” (when used in combination with CLR) in present example, corresponds to monomeric proteins, Reference to “trimeric spike” or “[S3]” (when used in combination with CLR) in present example, corresponds to a mix comprising monomeric, dimeric and trimeric protein complexes.

TABLE 1

| | | Mice | Beta spike | |
|---|---|---|---|---|
| Group | | (n = X) | Valence | Test |
| 1. | Naive | 5 | — | |
| 2. | CpG (2 μg) + Alum (500 μg) | 5 | — | Adjuvant control |
| 3. | Dimeric Beta spike [S2] | 5 | Dimeric | Dimeric spike without |
| | alone (10 μg) | | | adjuvant |
| 4. | [S2] (10 μg) + CpG (2 μg) | 10 | Dimeric | Dimeric spike with CpG |
| 5. | [S2] (10 μg) + Alum (500 μg) | 10 | Dimeric | Dimeric spike with Alum |
| 6. | [S2] (10 μg)+ Alum | 10 | Dimeric | Dimeric spike with both |
| | (500 μg) + CpG (2 μg) | | | adjuvants |
| 7. | Trimeric Beta spike [S3] | 10 | Trimeric | Trimeric spike with CpG |
| | (10 μg) + CpG (2 μg) | | | |
| 8. | [S3] (10 μg) + Alum (500 μg) | 10 | Trimeric | Trimeric spike with Alum |
| 9. | [S3] (10 μg) + Alum | 10 | Trimeric | Trimeric spike with both |
| | (500 μg) + CpG (2 μg) | | | adjuvants |

| | |
|---|---|
| Total | 75 |

At D+28, all the mice were sacrificed, Blood samples were collected at D−7, D+7 and at D+28 after the sacrifice to prepare serum. Spleno-lymphocytes were isolated from the spleens of the mice and stored frozen. The indicated numbers for the groups are used in subsequent graphs in present example 17.

Serological detection of IgGs (IgG1, IgG2a, IgG2b, IgG3) against SARS-CoV-2 using MSD assay (FIG. 21): V-Plex COVID-19 Coronavirus Panel 1 serology kits from MSD (reference K15362U) were used to detect the presence of IgG1, IgG2a, IgG2b and IgG3 antibodies to SARS-CoV-2-Spike, SARS-CoV-1 Spike, SARS-CoV-2 S1 NTD and SARS-CoV-2 S1 RBD in diluted sera from mice of the different injected groups. Serum dilution was 1/100 to detect anti-spike IgG2a, IgG2b, IgG3, and 1/500 to detect anti-spike IgG1. To detect these immunoglobulins, the protocol recommended by the manufacturer was followed, with adaptation for the SULFO-TAG anti-mouse antibodies of the mouse isotyping panel 1 kit (reference K15183B). The plate was read on an MSD instrument, which measures the light emitted from the MSD SULFO-TAG (ECL signals).

Determination of neutralizing antibody levels by MSD assay (FIG. 22 and data not shown): Multiplex assays for the detection of neutralizing antibodies against SARS-CoV-2 (SARS-CoV-2 Spike, SARS-CoV-2 S1 RBD and SARS-CoV-2 S1 NTD) were done on mice sera using the MSD V-Plex SARS-CoV-2 Panel 2 (ACE2) Kits from MSD (ref. K15386U) according to the manufacturer's instructions. For these inhibition tests, mice sera were diluted 1/50 (FIG. 22). The sera from the mice that received Dimeric beta spike and Trimeric beta spike were displayed by 2-fold serial dilutions in the 96-well plate to establish inhibition curves (data not shown). Data were recorded using a MESO Quick Plex SQ 120 instrument, which measures the light emitted from the MSD SULFO-TAG. Results were expressed as percent inhibition calculated using the equation below. Highly positive samples show high percent inhibition. % Inhibition=(1−Average Sample ECL Signal/Average ECL signal of calibrator 8)*100.

Neutralization assay using live Beta & Delta SARS-CoV-2 virus & VeroE6 target cells (data not shown): Vero-E6 cells were grown in DMEM containing 10% Fetal Bovine Serum (FBS) and 100 units/mL penicillin and 100 μg/mL streptomycin. Cells were incubated at 37° C. in 5% CO₂. SARS-CoV-2 viral isolates were obtained from nasopharyngeal samples and sequenced to determine the type of SARS-CoV-2 variant. Serum samples were heat-inactivated for 30 minutes at 56° C. Two-fold serial dilutions of sera were incubated with SARS-CoV-2 virus at 100 TCID₅₀ for 1 hour. Sera and virus mix was subsequently incubated with Vero-E6 cells for 60 hours at 37° C. The neutralization was assessed using the CCK-8 kit (Dojindo) to determine the percentage of cell survival compared to uninfected controls and no sera controls.

### Results

### Serological Detection of IgG1, IgG2a, IgG2b and IgG3 Against SARS-CoV-2 by MSD Assay (FIG. 21)

Titration of murine IgG1anti-SARS-CoV-2 Beta spike using MSD serology assay: Compared to the first vaccination campaign with the Dimeric Wuhan spike (see Example 8) where the signal was 10⁶ RU at serum dilution 1/100 with 3 mice out of 10 at lower signal (FIG. 12, IgG1CoV-2S, lane 12), the titrations of anti-SARS-CoV-2 Beta spikes IgG1from vaccinated mice showed strong saturating anti-spike IgG1 titres (above 10⁶ RU) at 1/500 dilution (FIG. 21 B, groups 3 to 9). The greatest signal homogeneity was observed in the group 6 (Dimeric Beta spike with Alum+CpG) and group 8 (trimeric Beta spike with Alum).

Furthermore, FIG. 21A shows that the anti-SARS-CoV-2 spike IgG1cross-reacts with the SARS-CoV-1 spike. The strongest, most homogeneous cross-reactive antibody titre was observed in group 6 (Dimeric Beta spike with Alum+CpG), compared to group 9. The signal increased from group 3 to group 6, the latest being the most homogeneous among the 10 mice. The optimal adjuvantization is the combination Alum+CpG (groups 6 & 9), followed by Alum alone (groups 5 & 8) and CpG alone (groups 4 & 7). The Dimeric spike is less immunogenic in the absence of adjuvant.

In addition, FIG. 21C&D concern anti-SARS-CoV-2 RBD & anti-SARS-CoV2 NTD IgG1. The strongest and most homogeneous signal was observed in mice of the group 6 (Dimeric Beta spike with Alum+CpG), more homogeneous than group 9 (trimeric Beta spike with Alum+CpG), with one mouse lower versus 4 mice lower, respectively (FIG. 21 D, comparison between groups 6 & 9).

FIG. 21E-H concern anti-SARS-CoV-2 IgG2a and show that the strongest signal was observed in groups 4 & 7 (F-H), and in the group 4 (Dimeric Beta spike+CpG) for anti-SARS-CoV1 S IgG2a (FIG. 21E). No difference was observed between groups 4 & 7 (Dimeric vs trimeric Beta spike+CpG) (FIG. 21F-H).

FIG. 21I-L concern anti-SARS-CoV-2 IgG2b. As for what was observed IgG2a, groups 4 & 7 also display the strongest IgG2b signal (FIG. 21J-L), although the differences between groups is smaller.

FIG. 21M-P concern anti-SARS-CoV-2 IgG3. Here again, the strongest signals are observed for sera from groups 4 & 7. According to the 3 last series of graphs, CpG alone (over Alum or combination Alum+CpG) elicits anti-SASR-CoV-2 IgG2a, IgG2b & IgG3 responses.

### Analysis of (i) the Concentration of Neutralizing Anti-SARS-CoV-2 S or RBD Abs and (ii) Their Neutralizing Capacity Using Respectively a MSD Serology & a SARS-CoV-2 Surrogate Virus Neutralization Assays (FIG. 22 A-J, FIG. 23)

### Material & Methods

Vero E6 cells were purchased from Vectorbuilder. They were cultured in DMEM with 10% FBS, 100 units/mL of penicillin and 100 μg/mL of streptomycin, Plasmids encoding the different SARS-CoV-2 spike variants (Wuhan, Beta, or Delta) were custom-synthesized by Vectorbuilder. All spike variants lack the last 19 C-terminal residues corresponding to an endoplasmic reticulum (ER)-retention signal in order to increase cell surface expression. pNBe3 and pHiBIT-N plasmids, encoding for the large part (LgBiT) and high-affinity small part (HiBiT) of Nanoluciferase (NanoLuc) were purchased from Promega.

Cell fusion assays based on the interaction of Spike with ACE2 were carried out as previously described, but using Nanoluciferase complementation [Buchrieser, J., et al., Syncytia formation by SARS-CoV-2-infected cells. EMBO J, 2021, 40(3): p. e 107405]. In brief, Vero E6 cells (3×10⁶), endogenously expressing ACE2 receptor and HEK 293T cells (6×10⁶) were plated in 10-cm culture dishes. 24 h later, Vero E6 cells were transfected with pHiBiT-N plasmid and HEK 293T cells were transfected with a SARS-CoV-2 spike variant (WT (i.e. Wuhan), Beta or Delta). 24 h after transfection, cells were detached using Versene (0.48 mM EDTA in PBS) and resuspended in DMEM with 10% FBS at a concentration of 1×10⁶ cells/ml.

For the assessment of neutralizing antibodies: Sera from vaccinated mice (as described in Example 17) or control sera were serially diluted in DMEM+10% FBS and co-incubated with 10⁴ Spike-expressing HEK 293T cells at 37° C. for 60 minutes before mixing with 6×10⁴ Vero E6 cells in a 96-well plate (FIG. 22 E-J).

For the assessment of the affinity of a soluble Spike protein towards ACE2: Soluble Spike was serially diluted in DMEM+10% FBS and co-incubated with 6.10⁴ Vero E6 cells at room temperature for 60 minutes before mixing with 10⁴ Spike-expressing HEK 293T cells in a 96-well plate (FIG. 23).

In both cases, after 16-20 h, supernatant was replaced with NanoLuc substrate diluted in Opti-MEM and luminescence was read immediately on a Promega GloMax Explorer for 20 minutes, Results are expressed as percentage of inhibition of syncytia formation, using a fusion-deficient Spike variant to define 100% inhibition.

Binding of anti-spike Abs from sera from SARS-CoV-2 recovered donors using ELISA with coated recombinant spikes (FIG. 23).

The different purified soluble recombinant spikes (monomeric Beta, CLR4, Dimeric Beta.CLR1, Dimeric Beta.C4bpβ, trimeric Beta.CLR5 & Dimeric Delta.CLR1) were coated at same concentrations in phosphate buffer saline (PBS) on Maxisorp 96-well ELISA plates for 24 h. After washing (PBS-1% BSA) and blocking (PBS-5% BSA) for 1h at 4° C. the ELISA plates were incubated with sera from 3 different recovered SARS-CoV-2 covid-positive individuals (data shown for #6 but not for the two other candidates) as 2×serial dilutions starting 1/50 in PBS-1% BSA for 1 h at 4° C. After washings, the plates were incubated with a goat anti-human IgG horseradish peroxidase (HRP)-conjugated for 1 h at 4° C. After washings, the plates were revealed with a mixture of o-phenylenediamine dihydrochloride (OPD) and H₂O₂, a chromogenic substrate for HRP. The oxidation reaction leading to a yellow-orange coloration was stopped with 0.5N H₂SO₄ solution. The plates were read at 492 & 630 nm using a spectrophotometer. It is noted that monomeric Beta.CLR4, Dimeric Beta.CLR1, Dimeric Beta.C4bpβ, trimeric Beta.CLR5 & Dimeric Delta.CLR1 were purified using a two-step purification process, such as described in Example 12 or 19.

FIG. 22A-B concern the concentration of neutralizing Abs against SARS-CoV-2 Wuhan spike or RBD fragment. In FIG. 22A, present inventors observed a statistically significant difference (p=0.00447) between groups 6 (Dimeric Beta spike) & 9 (trimeric Beta spike) in the presence of Alum+CpG, showing a higher and more homogeneous anti-SARS-CoV-2 spike neutralizing antibody response when using the Dimeric Beta spike over the trimeric counterpart. The titer of neutralizing Abs against SARS-CoV-2 RBD is also higher and more homogenous in the group 6 compared to the group 9.

MSD surrogate neutralization assay shown in FIG. 22C-D using vaccinated mice sera or mild or severe Covid-19-positive recovered patient sera (all diluted at 1/50) showed the strongest and most homogenous neutralizing response using sera from group 6 (Dimeric Beta spike+[Alum+CpG]). The neutralizing efficacy of antibodies generated in vaccinated group 6 with the Dimeric Beta spike is as good as for those from Covid-19-positive patients, and importantly very homogeneous in the ten vaccinated mice.

Evaluation of the Neutralization Efficacy of Elicited Anti-Beta Spike Abs in Sera from Vaccinated Mice Using SARS-CoV-2 Spike-Driven Syncytium Formation Assay (FIG. 22 E-F)

The present inventors tested the elicited antibody-mediated neutralization efficacy in a more physiological in vitro system compared to MSD, consisting of using a syncytium formation assay between Spike-expressing HEK293T cells and ACE2-positive VeroE6 cells. Using Promega NanoBiTR complementation system, cell fusion leads to LgBiT:SmBIT complex formation, nanoluciferase complementation and subsequent luminescence emission. Using this syncytium formation assay, present inventors analyzed antibody-mediated neutralization efficacy of syncytium formation, by measuring the serum dilution to get 50% inhibition or “half-maximum inhibitory serum dilutions”, which represents the “IC₅₀”. FIG. 22E-F depicts the results of analysis of neutralization of syncytium formation, using (E) WT spike- or (F) Beta spike-HEK293T effector cells and VeroE6 target cells.

FIG. 22E depicts the average IC₅₀ neutralization for the 10 individual mice sera from the Dimeric spike (1) and trimeric spike (2) mice groups, which are 1:450 & 1:50, respectively, making IC₅₀ cross-neutralization efficacy of antibodies produced in the Dimeric spike mice group 9-fold stronger than for antibodies produced in the Trimeric spike mice group. Regarding the control sera, IC₅₀ neutralization for the WHO standard control (3) and for the “CoV-Pos (Top 10/80)” convalescent donor (whose MSD neutralization antibody titres were ranked in the 10 best in a cohort of 80 convalescent individuals) (4) were 1:900 & 1:550, respectively. Comparing the neutralization efficacy of abs from the Dimeric spike group to the 2 controls, Abs in the Dimeric spike group display a neutralizing efficacy as good as the CoV-Pos (Top 10/80) serum and 2-fold lower than the WHO standard.

In FIG. 22F, average IC₅₀ neutralization for the 10 individual mice sera from the Dimeric spike (1) and Trimeric mice (2) mice groups were 1:1000 & 1:290, respectively, making IC₅₀ neutralization efficacy of antibodies produced in the Dimeric spike mice group 3.44-fold stronger than for antibodies produced in the trimer mice group, Regarding the control sera, IC₅₀ neutralization for the WHO (3) and CoV-Pos Top10/80 (4) sera were 1:380 & 1:35, respectively. Neutralizing Abs in the CoV-Pos (Top 10/80) serum do not cross-neutralize the Beta spike. In contrast, neutralizing Abs in the WHO standard serum still display a neutralizing potency, despite a drop by a factor 2.4.

Present inventors compared the difference of IC₅₀ neutralization efficacy between the beta Dimeric spike and trimeric spike groups (groups 1 & 2) in the first assay using WT spike-HEK293T (9-fold) & and in the second assay using Beta spike-HEK293T (3.44-fold). They concluded that neutralizing antibodies from Beta Dimeric spike mice group do cross-neutralize better the WT spike by a factor 2.61×(9/3.44), when compared to the neutralizing antibodies from the trimer mice group.

IC₅₀-differences between Dimeric spike & trimeric spike mice groups were statistically significant (p=0.047 in E & p=0.027 in F) as determined using two-tailed unpaired t-test.

Evaluation of the Neutralization Efficacy of Elicited Anti-Beta Spike Abs in Sera from Vaccinated Mice Using Authentic Beta (B.1.351) SARS-CoV-2 Virus (FIG. 22H-J)

Present inventors have analyzed the neutralization potency of antibodies in mice vaccinated with either the Beta Dimeric-spikes or the Beta trimeric-spikes using VeroE6 cells infected with authentic SARS-CoV-2 Beta variant. FIG. 22G summarizes the results of the experiment. Average IC₅₀ neutralization serum dilutions for Dimeric spike (1) & trimeric spike mice (2) group were 1:5000 & 1:900, respectively: Soluble Dimeric Beta spike vaccine elicit 5.55-fold greater neutralizing antibody titers than in mice vaccinated with the trimeric counterpart. Moreover, there is a higher homogeneity in the neutralizing response among the mice vaccinated with the Beta Dimeric spike, compared to the Beta trimeric spike mice group. In the latest, 2 mice did not elicit a neutralizing response (IC₅₀<20), while 2 mice elicited a very strong neutralizing responses, the IC₅₀ antibody titer was greater than 1:10.000 (highest dilution used in the experiment). The present inventors did not see a significant statistical difference between the Dimeric spike & trimeric spike mice groups, due to the high heterogeneity in the latest group. They have used 3 serum controls (groups 3-5 in FIG. 22G). (3) A serum from a convalescent donor infected with the Delta variant, (4) The Cov-Pos (Top 10/80) serum and (5) a serum from a donor collected 15 days after the second injection of the Pfizer anti-SARS-CoV-2 vaccine BNT162b (Pfz #2, D15). Their IC₅₀ neutralization serum titres were 1:2800, <1:20 & 1:650, respectively. Consistent with the previous experiment, the serum from the Cov-Pos (Top 10/80) donor did not neutralize the Beta variant. The serum from the convalescent donor infected with the

Delta variant displayed a strong cross-neutralization with the Beta variant, (i) lower by a factor 1.78 compared to the mouse serum vaccinated with the Beta Dimeric spike, and (ii) greater by a factor 3.11 compared to the mouse serum vaccinated with the Beta trimeric spike. At last, the IC50 neutralization serum titers of mice vaccinated with the Dimeric Beta spike was 7.7-fold greater than that of an individual vaccinated with BNT162b. This is in line with previous report showing that Gamma variant escaped from antibodies produced by BNT162b2 by a factor 5.8-fold (Garcia-Beltran, W. F. et al. 2021. Multiple SARS-CoV-2 variants escape neutralization by vaccine-induced humoral immunity. Cell 184, 2523). Since both Beta & Gamma variants share the E484K RBD substitution, this suggests that this mutation could also be a determinant for low neutralization of post-vaccinated sera against Beta variant.

### Generation of Soluble Recombinant Dimeric Delta (B.1.617.2) & Omicron (B.1.1.529) Spike

The 2 successive in vivo vaccination experiments allowed present inventors to identify an optimized protein complex based on the CLR1 scaffold to elicit a strong neutralizing antibody response, successively excluding the monomeric ([S]¹) protein complexes based on the CLR1 scaffold and the trimeric protein complexes based on the CLR2 scaffold. They further focused on the production and study of other Dimeric spikes, generating the soluble recombinant Delta.CLR1 and Omicron.CLR1 spikes with the optimized production process.

The amino acid (SEQ ID NO: 45 and 47) and nucleic acid (SEQ ID NO: 44 and 46) sequences of the Delta.CLR1 and Omicron.CLR1 synthetic gene cloned in pEF-IRESpac expression vector are represented in FIG. 4.

It is noted that Delta.CLR1 and Omicron.CLR1 were purified using a two-step purification process, such as described in Example 12 or 19.

Moreover, in order to explore the influence of CLR1 dimerization scaffold on the folding and dimeric association of 2 monomeric spike, the authors used an alternative control dimerization scaffold: The 58 residues from the C-terminal domain of the C4bp β-chain (C4bpβ) to express dimeric Beta spikes. In contrast to CLR1, which displays a single cysteine, C4bpβ displays 2 cysteines. In the native structure, a single C4bpβ-chain is covalently anchored to the heptameric C4bpβ core to complete the C4bp quaternary structure, When C4bpβ is fused in C-terminal of a soluble peptide, the peptide is secreted as dimers.

Although C4bpβ and CLR1 have about the same length, CLR1 displaying a single cysteine may likely present an increased flexibility compared to C4bpβ, which may influence the dimeric association. Present inventors have thus produced Beta, C4bpβ spike glycoproteins.

Binding of Anti-Spike Abs from Convalescent Donors to 5 Different Recombinant Soluble Coated Spikes Using ELISA

The present inventors explored to which extent there would be a relationship between the relative affinity these spikes for ACE2 and their ability to capture anti-spikes antibodies generated in serum from 4 individuals previously infected, who elicit various titres of anti-spike antibodies (#3>#6>#2>#10). Same amount of each spike were coated to ELISA plates, and serial dilution of sera of the 4 donors were incubated to the ELISA plates. The ELISA plates were revealed with an anti-human IgG pAb HRP-conjugated. The dose-response curves were represented as % initial signal with lowest serum dilution which gives a saturating signal in all 4 sera. FIG. 23A represents an example of dose-response curves for serum #6 (donors #3, #2 and #10 not shown). For each molecule, the serum dilution for half-maximum binding (EC₅₀) was determined for each of the 4 sera. Using the EC₅₀ for soluble Dimeric Beta spike (CLR1) as reference, the EC₅₀ ratios was calculated for a given spike divided by Beta Dimeric spike EC₅₀. Ratios indicate the fold-binding capacity a given spike to capture anti-spike antibodies from sera compared to Dimeric Beta spike (CLR1). EC₅₀ ratios allow ranking the different soluble spikes for their capacity to capture anti-spikes antibodies related to Beta spike.CLR1 for the 4 sera, regardless anti-spike antibody titres, which vary considerably between the 4 convalescent sera. The results depicted in FIG. 23B show that the Beta spike.CLR1 is the spike with highest binding capacity. Dimeric Delta & omicron spike show a binding capacity for anti-spike Abs that is 25% & 33% lower than Dimeric Beta spike (CLR1), respectively. For one of the 4 sera, the binding capacity to Delta Dimeric spike was slightly superior to Dimeric Beta spike (CLR1), probably because the convalescent donor has been infected with the Delta variant. Trimeric Beta spikes show a binding capacity that is more than 50% lower than Beta.CLR1 Dimeric spikes. At last, binding capacity for anti-spike antibodies to dimeric Beta spike.C4bpβ show 70% reduction in binding capacity for anti-spike antibodies compared to Dimeric Beta spike.CLR1.

Present inventors' CLR1 allows the expression of multimeric protein complexes with a suitable folding keeping the correct cryptic epitopes in the dimeric protein complexes that are recognized by the anti-spike Abs from the sera. These precious cryptic epitopes may be hidden in the multimeric protein complexes based on the CLR2 scaffold, limiting the capacity to elicit a strong neutralizing immune response.

Together, present inventors data seem to show that the soluble recombinant dimeric spike (Beta or from another variant) using present inventors' CLR1 scaffold may be an ultimately optimized structure as subunit vaccine candidate, Compared to its trimeric counterpart, soluble recombinant spike: has an enhanced affinity for ACE2 compared to the trimeric spike counterpart; binds anti-spike Abs with higher efficacy; and as immunogen, leads to a strong and more homogeneous neutralizing immune response in immunized mice

As further explained in Example 20, present inventors have found using cryo-EM that the dimeric protein complexes based on the CLR1 scaffold are in fact trimeric spike protein complexes in a post-fusion stabilized state; the monomeric proteins based on the CLR1, CLR5 or CLR4 scaffold are in fact trimeric spike protein complexes in a pre-fusion stabilized state and the trimeric protein complexes based on the CLR2 or CLR5 scaffold are presumed oligomers of the trimeric post-fusion-stabilized spikes. Accordingly, the “Dimeric spike” (e.g. Dimeric Beta Spike, Dimeric Delta Spike or Dimeric Omicron Spike), “dimeric ([S2])” or “[S2]” based on the CLR1 scaffold as referred to in present example are in fact a combination of pre-fusion trimeric spikes and post-fusion trimeric spikes, the “monomeric spike”, “monomeric ([S1])” or “[S1]” based on the CLR4 scaffold as referred to in present example are in fact pre-fusion trimeric spikes, and the “trimeric spike ([S3])” based on the CLR2 scaffold as referred to in present example are in fact a combination of pre-fusion trimeric spikes, post-fusion trimeric spikes, and presumed oligomers of post-fusion trimeric spike subunits, preferably predominantly comprising the presumed oligomers of post-fusion trimeric spike subunits.

The 2-step purification (His-Trap & gel filtration) dramatically enriched in post-fusion form (previously referred to as “dimers”) (representing approximately 80%). The mix comprises approximately 20% pre-fusion form (previously referred to as “monomers”). It was a surprise to present inventors to see that most of the ‘dimer’ sample that led to the induction of a strong, homogeneous immune response corresponds in fact to post-fusion stabilized-trimers. That would certainly explain exposure of neo-epitopes in this construct. It is of high interest to think that the improved observed neutralization would be because of epitopes specific to the post-fusion conformation of the trimeric spike protein.

### Example 18

### Assessment of the Protection Conferred by Vaccination with the Recombinant Dimeric SARS-CoV-2 Beta Spike Glycoprotein Using K18-hACE2 Mice Vaccinated and then Challenged Through Intranasal Infection with the β Variant of SARS-CoV-2 (FIG. 24)

### Material & Methods

Study P2145 made by the C.R.O. Voxcan: K18-hACE2 transgenic mice expressing human ACE2 [B6.Cg-Tg(K18-ACE2)2Prlmn/J] were used for the vaccination/challenge experiment. The sensitization was performed as described in FIG. 12.

At day 0, a first group (n=10) received a first dose of 10 μg His-Trap, gel filtration-purified soluble recombinant Dimeric Beta Spike [with CLR1 molecular scaffold]+combined adjuvants [500 μg Alum+2 μg CpG] (through intraperitoneal administration or IP), (ii) a second group of n=10 mice received the 2 combined adjuvants alone [Alum+CpG] and (iii) a third group of n=3 mice received nothing and was not infected. At day +14, the first group received a second dose of the Dimeric Beta spike +[Alum+CpG] and the second received the combined adjuvants [Alum+CpG].

At D+28, the first 2 groups were infected by the SARS-CoV-2 Beta variant [lot UVE/SARS-CoV-2/2021/FR/1299-ex SA (lineage B 1.351) at 10² PFU/50 μl]: The mice were instilled with 50 μl of prepared SARS-CoV-2 Beta variant suspension, equally distributed into each nostril of mice, using a thin pipette cone. The body weight (BW) was measured at D0 (reference BW), then once a week between D0 and D+28 (D0, D+7, D+14, D+21 & D+28). From D+28 to D+42, end of the experiment, the BW was measured daily. The body temperature (BT) was measured daily from D+28 until D+42. The clinical follow up (using a scoring grid) and the total score evolution was also performed daily from D+28 until D+42. The total score is a representation of the severity of the observed clinical signs, including general aspects such as piloerection, hunched back, lack of grooming, and ocular/nasal discharge; eyes; mobility; respiratory signs; and other signs such as tremor, diarrhea and vocalization when handled. Following each clinical follow-up evaluation, the total score will be calculated by the sum of the scores linked to the observed clinical signs.

### Results

Protective Efficacy evaluation of the Lead Soluble Recombinant Dimeric Beta Spike Protein Subunit Vaccine in K18-hACE2 Transgenic Mice (FIG. 24)

Present inventors' data showed a protective efficacy of the lead vaccine compound: FIG. 24A shows the individual mouse body weight evolution for (i) the uninfected control group, (ii) the infected group that was not vaccinated & (iii) the infected group that was vaccinated. The curves are expressed as percentage mouse initial body weight at D28, day of the first injection. Nine mice out of 10 have lost weight in the non vaccinated group, while all the vaccinated mice kept a stable weight (Table 2).

TABLE 2

| D35 | No weight loss | Weight loss |
|---|---|---|
| Infected | 1 | 9 |
| Vaccinated/infected | 10 | 0 |

Fisher's exact test;
p = 0.000119

TABLE 3

| Death | Found dead | (>20% weight loss) | survival |
|---|---|---|---|
| Infected | 4 | 3 | 3 |
| Vaccinated/infected | 0 | 0 | 10 |

In vaccinated K18-hACE2 transgenic mice that were challenged at D28 with the SARS-CoV-2 Beta variant and followed-up until D42, none of them died (FIG. 24B and Table 3), they almost lost no weight (FIG. 24A and Table 2), and their total score was 0 (data not shown). In contrast, in the non-vaccinated group, 4 mice were found dead, and 3 had to be early terminated (Table 3) because they had a very bad total score and lost more than 20% weight (Table 2) and were in a bad shape. The average body temperature in the vaccinated group had a slight increase of less than 1° C. between the day of infection and the D35, coming back to normal from D35 to D42 (data not shown). In FIG. 24A, body weight change was expressed as “percentage of body weight change”, taking as reference de D28, day of challenge with the SARS-CoV-2 Beta virus. The results of statistical analysis using the Tukey's multiple comparison test between the 3 groups showed a significant statistical difference in body weight change (%) between the non-vaccinated/infected and the vaccinated/infected groups of p<0.0374 (*).

Among the 3 unvaccinated and challenged mice that survive, 2 did lose weight from the day of infection (D28) until D35. The third mouse did not lose weight. In a statistical point of view, if present inventors consider the D35 were 9 mice out of 10 in the non-vaccinated group versus 0 in the vaccinated group, the Fisher's exact test gives a score p=0.000119, which is highly significant (Table 2).

In term of death, the non-vaccinated group displayed 4 dead mice, 3 early-terminated mice versus 0 dead in the vaccinated group. The Fisher's exact test gives a score of p=0.00309, which is also highly significant.

As further explained in Example 20, present inventors have found using cryo-EM that the “dimers” as referred to in present example are in fact trimeric spike protein complexes in a post-fusion stabilized state.

### Example 19

### Purification of Soluble Recombinant SARS-CoV-2 Spike Glycoproteins (FIG. 25) (Two-Step Purification with First His-Trap and Secondly Gel Filtration by Size-Exclusion Chromatography)

### Material & Methods

His-Trap purification using one-step imidazole elution: A 1 ml Excel Nickel His-Trap column was connected to a peristaltic pump and the filtered opti-MEM supernatant from HEK 293T expressing the SARS-CoV-2 spike variants was passed through the column for 5 days as a closed-loop (flow rate 2 ml/min) in order to immobilise the soluble mono/dimeric/trimeric recombinant SARS-CoV-2 spike glycoproteins to the column.

The column was connected to a FPLC system and an elution buffer (20 mM phosphate, 500 mM NaCl, 1M imidazole) was applied to eluate the soluble recombinant SARS-CoV-2 mono-/multi-valent spikes. The eluate was concentrated using 15 ml Centricon (centrifugal PVDF filter devices) with 50-100 kDa molecular weight cut-off (MWCO).

Gel-filtration using 2% glycerol eluent: A Superdex 200 increase 10/300 GL column was connected to a FPLC. Concentrated His-Trap eluate was injected onto the top of the column. The protein separation by size was performed in a 2% glycerol eluent (flow rate 0.1 ml/min).

Dimeric/trimeric recombinant SARS-CoV-2 Beta spike glycoproteins were eluted in 0.5 ml fractions from 7 to 9.6 ml and monomeric fractions from 9.6 to 11 ml. Dimeric spikes from Delta and Omicron variants were isolated using the same 2 step procedure (His-Trap purification followed by Gel-filtration).

Eluted fractions were analysed using SDS-Page followed by a silver staining, Fractions of same protein conformation were pooled and concentrated using centricon with 50 kDa MWCO.

### Results

Purification Patterns of the Monomeric, Dimeric and Trimeric Spikes after His-Trap Gel Filtration Purification and analysis Using SDS-PAGE and Silver Staining (FIG. 25)

The silver staining are generally accompanied with the related chromatograms depicting the analyzed fractions for the purification of A, the monomeric form, B, the dimeric form and C, the trimeric form of the Beta spike.

In the first generation of produced molecules, only His-Trap purification was used with stepwise imidazole elution to enrich in dimeric forms.

In the course of present inventors' vaccine development process, present inventors have dramatically improved the production and purification process, which has contributed to constantly increase the quality of the manufacturing process.

In the second generation of production of soluble recombinant oligomeric spikes, present inventors have combined a one-step elution after His-Trap capture, a step of concentration and gel exclusion purification using a S200 sephadex size-exclusion column.

Although the resolution of the gel extraction column is not completely optimal for such high MW molecules, and does not always allow to completely separate the molecular species from each other, when CLR 1 is used (B.), only dimers and monomers are present (traces of trimers), and the enrichment in dimeric form is very efficient. Moreover, during the gel filtration process, present inventors get rid of all far less immunogenic spike degradation products. The same CLR1 scaffold was combined to the sequence of the spike from variants Delta and Omicron to purify respective spike proteins via the 2 step upgraded protocol of purification (His-Trap affinity followed by gel exclusion on S200 Sephadex column).

When CLR5 is used, there are trimeric, dimeric and monomeric forms present, and the gel filtration allows removing a large majority of non-immunogenic monomeric forms, all the degraded forms, and a large part of the dimeric form.

As further explained in Example 20, present inventors have found using cryo-EM that the “dimers” as referred to in present example are in fact trimeric spike protein complexes in a post-fusion stabilized state; and the “monomers” as referred to in present example are in fact trimeric spike protein complexes in a pre-fusion stabilized state.

### Example 20

### Structural Analysis of the “Monomeric” and “Dimeric” Spikes Using Cryo-Electron Microscopy (Cryo-EM)

SARS-CoV-2 is the third beta-coronavirus after SARS-CoV-1 & MERS-CoV to be transferred to humans in the 21st century, and given the large natural reservoir of similar viruses in species such as bats1, another pandemic caused by a new coronaviruses is likely to happen again.

Potent neutralizing antibodies (nAbs) against several epitopes on SARS-CoV-2 Spike glycoprotein have been identified in convalescent patients & the RBD of SARS-CoV-2 Spike glycoprotein is an immunodominant and highly specific target of 90% of the neutralizing activity present in SARS-CoV-2 immune sera. RBD is also the main target of serum neutralizing activity in vaccinated individuals and comprises several antigenic sites recognized by nAbs with a range of neutralization potencies and breadth. Anti-spike and RBD antibody responses correlate with the severity of the symptoms, Virus neutralization titres in patients' sera are highly correlated with the levels of IgG directed against conformational discontinuous but not sequential linear RBD epitopes, and the majority of virus-neutralizing activity in sera of SARS-CoV-2 patients can thus be attributed to antibodies against conformational RBD epitopes. In contrast, elicited Abs against linear RBD have no neutralizing activity. Therefore, the majority of current vaccines to SARS-CoV-2 are mainly based on the use of a prefusion state-stabilized spike (SARS-CoV-2 2P S (Hsieh, S. M. et al. Safety and immunogenicity of a Recombinant Stabilized Prefusion SARS-CoV-2 Spike Protein Vaccine (MVC-COV1901) Adjuvanted with CpG 1018 and Aluminum Hydroxide in healthy adults: A Phase 1, dose-escalation study. EClinicalMedicine 38, 100989 (2021)), HexaPro S (Hsich, C. L. et al, Structure-based design of prefusion-stabilized SARS-CoV-2 spikes. Science 369, 1501-1505 (2020)), RCC3/RCC6 (Riley T. P. et al. Enhancing the Prefusion Conformational Stability of SARS-CoV-2 Spike Protein Through Structure-Guided Design. Frontiers in immunology 12, 660198 (2021))) aiming at inducing antibodies against the RBD that eventually block the interaction with the host receptor. However, if the RBD is one of the main domains to induce neutralizing antibodies, it is also the domain with the highest variability rate and subject to mutations during new variant outbreaks. Consequently, RBD-specific antibodies are limited by the variability of the RBD among the new evolving variants. The efficacy of current vaccines based on the highly variable pre-fusion state-stabilized spike may rapidly be altered or impaired. In contrast, S2 is more conserved among coronaviruses than S1. SARS-CoV-2 HR1 & HR2 domains display 88% & 100% homology with SARS-CoV HR1 and HR2 domains, respectively. Therefore, S2 as a potential immunogen is worthwhile to consider, in the manner of previously characterized broadly neutralizing Abs against gp41 MPER in HIV-1 (i.e. 2F5, 4E10, 10E8 nAbs).

Recently isolated antibodies capable of cross-neutralizing human coronaviruses bind to the conserved stem helix region on S2 (residues 1140-1165), reviving hopes for pan-coronavirus vaccines.

Antibodies induced against constructs as taught herein, and more particularly the highly conserved post-fusion state-stabilized S2, (i) may induce broader neutralizing, longer-lasting antibody response, (ii) may reduce the likelihood of sequence altering mutations that render the immunogen ineffective, thus enhancing the cross-reactivity potential not only against SARS-CoV-2 current and future variants but also against other future coronaviruses. Ideally, a protein-based vaccine combining prefusion and post-fusion state-stabilized spikes would likely elicit broad spectrum cross-reacting neutralizing Abs.

### Materials and methods

### Purification 1: His-Trap Purification Using One-Step Imidazole Elution:

A 1 ml Excel Nickel His-Trap column was connected to a peristaltic pump and filtered optiMEM supernatant was passed through the column for 5 days as a closed-loop (flow rate 2 ml/min) in order to immobilise soluble SARS-CoV-2 spike glycoproteins to the column.

The column was connected to a FPLC and 1M imidazole solution was applied to eluate the mono/dimeric/trimeric spikes. The eluate was concentrated using centricon with 100 kDa molecular weight cut-off (MWCO).

Purification 2: His-Trap Purification Products were Further Purified Using Size-Exclusion Chromatography (SEC or Gel-Filtration) Using 2% Glycerol Eluent:

A Superdex 200 increase 10/300 GL column was connected to a FPLC, Concentrated His-Trap eluate is applied on the column. The protein separation by size was performed in a 2% glycerol eluent (flow rate 0.1 ml/min).

Dimeric/trimeric recombinant SARS-CoV-2 spike glycoproteins were eluted in 0.5 ml fractions from 7 to 9.6 ml and monomeric fractions from 9.6 to 11 ml.

### Analysis of the Purified Spike Oligomers Using SDS-PAGE:

Eluted fractions were analysed using a 4-15% acrylamide gel under non-reducing conditions. SDS-PAGE was followed by a silver staining. Fractions of same protein conformation were then pooled & concentrated using Centricon with 50 kDa molecular weight cut-off (MWCO) in order to prepare the final batches used for mice immunization.

### Cryo-EM Data Collection and Processing:

Purified “dimer sample” were placed on a glow-discharged holey carbon grid (Quantifol, Au 400 mesh). The grid was flash plunged into liquid ethane using an automatic plunge freezer (Leica EMGP2) operated at 10° C. and 75% humidity.

In the initial particle picking, 17351 spike particles originating from about 70 micrographs identified in 2D classification were used to train a custom Topaz model. Qualitative assessment identifies the presence of pre-fusion spike trimers as well as post-fusion spike trimers in the B.1.351 “dimeric spike” sample (Beta.CLR1). From these 17351 spike particles, 4614 were trimeric pre-fusion-stabilized spikes (26.6%) and 12737 were trimeric post-fusion-stabilized spikes (73.4%). A true quantitative assessment of ratios of particle conformations by cryo-EM is not possible: (i) particles can have different efficiency in populating the holey ice/carbon, and (ii) picking model can alter the efficiency with which certain conformations are picked-up. However, semi-quantitative assessment clearly suggests an excess of post-over pre-fusion spike (B1.351 Beta variant) at a ratio of 2:1 to 3:1.

### Results

Present inventors found using cryo-EM that
the Beta.CLR1 “dimers” (CLR1 scaffold using a single cysteine C52), and the “dimers” as referred to in examples 1-19 above in general, are in fact trimeric spikes in pre- or post-fusion stabilized states. More particularly, i) the minor (˜25%) lower band (˜200 apparent MW) and (ii) the major (˜75%) upper band (˜300 apparent MW) observed in FIG. 26 D (middle panel), corresponds to (1) post-fusion- and (2) pre-fusion-stabilized trimeric beta spike, respectively. Accordingly, the “dimers” as referred to throughout the present specification are in fact trimeric protein complexes, not dimeric protein complexes:
the Beta.CLR4 “monomers” (highly flexible CLR4 scaffold lacking cysteine), and the “monomers” as referred to in examples 1-5, 7, 10, 12-17 and 19 above in general, correspond to a single molecular species displaying only pre-fusion stabilized trimeric spikes (see FIG. 26 D (left panel))). Accordingly, the “monomers” as referred to throughout the present specification are in fact trimeric protein complexes, but in a pre-fusion stabilized state.
The “beta trimer” sample, using the more rigid CLR5 scaffold containing 3 cysteines (C52, C98 & C105), leads to the expression of 3 molecular species, the lowest and intermediate ones corresponding to the 2 molecular species of the “beta dimer” sample, and a third additional molecular species with higher band (˜600 kDa MW) that would correspond to post-fusion-stabilized trimeric spike subunits that present higher degree of oligomerisation, thanks the presence of the 3 cysteines, as observed in the cryo-EM micrograph analysis (data not shown).

The results show that the modulation of the cysteines within the CLR scaffold modifies the intrinsic flexibility/rigidity of the CLR. This modulation of the flexibility/rigidity of the CLR inflicts particular structural constraints in the trimeric spike subunits, leading to subsequent stabilization under particular folding:
A more flexible CLR scaffold lacking cysteine (CLR4) stabilizes the trimeric spike subunits exclusively under pre-fusion state.
The addition of a single cysteine (e.g. C52) in the CLR (CLR1) stabilizes the trimeric spike subunits either under pre-fusion or under post-fusion state. The size-exclusion chromatography (SEC) does not allow to completely separate the pre-fusion trimeric spike from the post-fusion trimeric spikes, but allows a dramatic enrichment of the post-fusion spike in the earlier elution fractions to a ratio 1:3 (post-/pre-fusion spikes).
The addition of two additional cysteines (e.g. C98, C105 in addition to C52) in the CLR (CLR5) leads to a more rigid scaffold, which generates an additional higher MW molecular species. This higher form would be due to the formation of disulfide-bridges between several trimeric spike subunits.

The B.1.351 “dimeric spike” corresponding in fact to the post-fusion-stabilized trimeric spike subunits, exposes neo-epitopes that are likely shielded in the more classical formulations of the spike protein or its fragments. The improved neutralization observed in sera from mice immunized with the B.1.351 “dimeric spike” sample would be because of epitopes specific to the post-fusion conformation of the spike protein that elicits such neutralizing Abs.

### CONCLUSION

Present inventors have used a novel self-trimerizing peptide scaffold, the collagen-like region (CLR) of ficolin-2, preferably human ficolin-2 (15-fold G-X-Y), which was C-terminally fused to the extracellular domain of SARS-CoV-2 spike—allowing stabilizing produced trimeric soluble recombinant SARS-CoV-2 spike under pre- or post-fusion state, without the need of introducing any stabilizing proline substitution(s). The only modification made in the SARS-CoV-2 spike used in the examples is the “GSAS”

(SEQ ID NO: 13) substituted at the furin cleavage site (residues 682-685), as described elsewhere in the present specification.

The modulation of cysteines within CLR dictates the preferred trimeric stabilized-state of the spike:
CLR1: The use of original CLR (CLR1) that contains a single cysteine in position C52 in the first G-X-Y repeat leads to the co-expression of 2 molecular species: trimeric pre-fusion (also referred to herein as “monomers”) and post-fusion (also referred to herein as “dimers”) stabilized-spikes. His-Trap purification followed by size-exclusion chromatography considerably enriched in post-fusion stabilized-spike. For example, transfection of the CLR1 construct into HEK293 cells as shown in FIG. 2 leads to expression of “[S]²”, which represents a co-expression of trimeric pre-fusion and post-fusion stabilized spikes.
CLR4: The absence of cysteine in the CLR (cysteine-free CLR4) leads to the exclusive expression of a single pre-fusion stabilized-spike: This single band corresponds to the lower band observed in the 2-band molecular pattern observed in the above construct with CLR1. For example, transfection of the CLR4 construct into HEK293 cells as shown in FIG. 2 leads to expression of “[S]¹”, which represents expression of a pre-fusion stabilized spike.
CLR5: The use of a 10 residue C-terminal extension of CLR1 towards fibrinogen-like region (FLR) of ficolin-2 including an overall 3 cysteines (C52, C98 & C105), called CLR5, leads to a purified spike with 3 molecular species. The 2 similar molecular species observed when using CLR1, plus an upper molecular species, which corresponds presumably, without to be bound by theory, to oligomers of trimeric post-fusion-stabilized spikes. For example, transfection of the CLR5 construct into HEK293 cells as shown in FIG. 2 leads to expression of “[S]³”, which represents expression of 3 molecular species, namely the trimeric pre-fusion stabilized spikes, the post-fusion stabilized spikes and the presumed oligomers of trimeric post-fusion-stabilized spikes.

In view of the above, present inventors demonstrated that the presence or absence of a cysteine in the CLR, as well as the number of cysteines in the CLR, modulates the state of trimeric spike. More particularly, when no cysteines are present in the CLR (e.g. CLR4), the trimeric spikes are present in a pre-fusion state, while when one cysteine is present (e.g. CLR1 with a single cysteine in position X of the first G-X-Y repeat (C52)), the trimeric spikes are present in both the pre-and post-fusion state, but predominantly in the post-fusion state.

1. A multimeric protein complex comprising three polypeptides each comprising N- to C-terminally:
(i) a receptor-binding domain (RBD) of an S1 subunit of a Spike (S) protein of a coronavirus,
(ii) optionally a S2 subunit of an S protein of a coronavirus; and
(iii) a multimerization domain comprising a collagen-like region (CLR) of ficolin-2, wherein the multimerization domain enables the assembly of the polypeptides into a trimeric protein complex,

2. The multimeric protein complex according to claim 1, wherein the multimerization domain comprises at most 75 amino acids.

3. The multimeric protein complex according to claim 1, wherein the multimerization domain comprises from 1 to 3 cysteines.

4. The multimeric protein complex according to claim 1, wherein the multimerization domain consists of (i) the CLR of ficolin-2; or (ii) the CLR of ficolin-2 and immediately C-terminally of the CLR of ficolin-2 a peptide consisting of three amino acids of which one is a cysteine, optionally wherein the peptide corresponds to the first three amino acids of the fibrinogen-like region (FLR) of ficolin-2.

5. The multimeric protein complex according to claim 1, wherein the polypeptides each comprise a linker peptide C-terminally of the S2 subunit of the S protein of the coronavirus and N-terminally of the multimerization domain.

6. The multimeric protein complex according to claim 1, wherein at least one of the polypeptides comprises at its C-terminal end a tag, optionally wherein the tag comprises N-terminally a proteolytic cleavage site.

7. The multimeric protein complex according to claim 1, wherein the polypeptides each comprise the complete S1 subunit and the S2 subunit of the S protein of the coronavirus.

8. The multimeric protein complex according to claim 7, wherein the S1/S2 cleavage site is mutated, thereby preventing proteolytic processing of S protein in the S1 and S2 subunits.

9. A polynucleotide encoding a polypeptide of the multimeric protein complex according to claim 1.

10. The polynucleotide according to claim 9, wherein the polynucleotide does not comprise a sequence encoding the signal peptide or part of the signal peptide of the coronavirus S protein.

11. An expression vector comprising the polynucleotide according to claim 9.

12. A method for preparing a trimeric protein complex, comprising
(a) introducing a polynucleotide encoding a polypeptide comprising N- to C-terminally:
(i) a receptor-binding domain (RBD) of an S1 subunit of a Spike (S) protein of a coronavirus,
(ii) optionally a S2 subunit of an S protein of a coronavirus; and
(iii) a multimerization domain comprising a collagen-like region (CLR) of ficolin-2, wherein the multimerization domain enables the assembly of the polypeptides into a trimeric protein complex, into a host cell,
(b) allowing the host cell to express and secrete the polypeptides, resulting in the self-multimerization of the polypeptides into trimeric protein complexes; and
(c) separating the trimeric protein complexes from the supernatants.

13. A trimeric protein complex obtainable by or obtained by the method according to claim 12.

14. A composition comprising a combination of protein complexes, the protein complexes comprising three polypeptides, each comprising N- to C-terminally:
(i) a RBD of an S1 subunit of an S protein of a coronavirus,
(ii) optionally a S2 subunit of an S protein of a coronavirus; and
(iii) a multimerization domain comprising a CLR of ficolin-2, wherein the polypeptides have not assembled, or the polypeptides have assembled into trimeric protein complexes by way of said multimerization domain.

15. A pharmaceutical composition comprising the multimeric protein complex according to claim 1, and a pharmaceutically acceptable carrier.

16. A vaccine comprising a multimeric protein complex according to claim 1.

17. (canceled)

18. A method of preventing a coronavirus infection in a subject, the method comprising administering to said subject, the multimeric protein complex according to claim 1 so as to prevent a coronavirus infection.

19. The method according to claim 18, wherein said coronavirus infection is a Severe Acute Respiratory Syndrome Coronavirus-2 (SARS-CoV-2) infection.


SEQUENCE LISTING (47 sequences)

SEQ ID NO: 1 - protein, 6 aa, synthetic construct
SGGGGS

SEQ ID NO: 2 - protein, 1300 aa, synthetic construct
LANSPVAAMGAGATGRAMDGPRLLLLLLLGVSLGGARSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTILRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPIDSGGGGSGCPGLPGAPGPKGEAGTNGKRGERGPPGPPGKAGPPGPNGAPGEPTSFEHHHHHHHH

SEQ ID NO: 3 - protein, 8 aa, synthetic construct
HHHHHHHH

SEQ ID NO: 4 - DNA, 3912 bp, synthetic construct
  source 1..3912: mol_type = other DNA
gctagcgaattcccccgtggccgccatgggcgccggagctaccggaagagccatggatggccccagactgctgctgctgctcctgctgggcgtgagcctgggcggagctagatccagccagtgtgtgaacctgacaaccaggacacagctgccccccgcctacaccaatagctttaccaggggcgtgtactaccccgacaaggtgtttaggtcctccgtgctgcacagcacacaggatctgttcctgccctttttctccaacgtgacatggttccacgccatccacgtgtccggcacaaatggcaccaagagattcgacaaccctgtgctgccctttaatgatggcgtgtactttgccagcaccgagaagtccaacatcatcaggggctggatcttcggcacaaccctggattccaagacacagtccctgctgatcgtgaacaacgccaccaatgtggtcattaaggtgtgtgagttccagttctgtaatgaccccttcctgggcgtgtactatcacaagaacaataagagctggatggagtccgagtttagggtgtacagcagcgccaacaattgcacatttgagtacgtgtcccagcccttcctgatggatctggagggcaagcagggcaactttaagaacctgagagagttcgtgttcaagaacatcgacggctactttaagatatattccaagcacacccccatcaacctggtgagggacctgcctcagggcttttccgccctggagcccctggtggacctgccaatcggcatcaatatcaccaggtttcagacactgctggccctgcacaggtcctacctgacccctggcgactccagctccggctggacagccggagccgctgcttactacgtgggctacctgcagcctaggacctttctgctgaagtacaacgagaatggcaccatcacagatgccgtggactgcgccctggaccccctgtctgagacaaagtgcaccctgaagtccttcaccgtggagaagggcatctaccagacctccaacttcagagtgcagcctaccgagagcatcgtgaggttccctaacatcacaaacctgtgccccttcggcgaggtgttcaatgccacaagattcgcctccgtgtacgcctggaataggaagaggatcagcaactgcgtggccgactactccgtgctgtacaacagcgcctccttttccacctttaagtgctacggcgtgagccctaccaagctgaacgacctgtgcttcacaaacgtgtacgccgatagctttgtgatcaggggcgatgaggtgagacagatcgcccccggccagaccggcaagatcgccgattacaactacaagctgcccgatgactttaccggctgcgtgatcgcctggaatagcaataacctggatagcaaggtgggcggcaactacaattacctgtacaggctgttcagaaagagcaatctgaagccttttgagagggatatctccacagagatatatcaggccggctccaccccttgcaatggcgtggagggctttaattgctacttccctctgcagagctacggctttcagcccaccaacggcgtgggctaccagccctacagagtggtggtgctgtcctttgagctgctgcacgcccccgccacagtgtgtggcccaaagaagagcaccaacctggtgaagaataagtgcgtgaattttaacttcaacggcctgacaggcacaggcgtgctgaccgagtccaataagaagtttctgcctttccagcagttcggcagggacatcgccgacaccacagacgccgtgagagatccccagaccctggagatcctggacatcaccccctgcagcttcggcggcgtgtccgtgatcacccctggcacaaacacctccaaccaggtggccgtgctgtaccaggatgtgaactgcaccgaggtgcctgtggccatccacgccgatcagctgacccctacctggagagtgtactccacaggctccaatgtgtttcagacaagagccggctgcctgatcggcgccgagcacgttaacaacagctacgagtgtgacatccctatcggcgccggcatctgtgcctcctaccagacacagacaatcctgagaagcgtggccagccagtccatcatcgcctacaccatgagcctgggcgccgagaatagcgtggcctactccaataatagcatcgccatccccaccaacttcacaatcagcgtgacaacagagatcctgcccgtgtccatgacaaagacatccgtggattgtacaatgtacatctgcggcgattccaccgagtgtagcaatctgctgctgcagtacggctccttttgtacccagctgaacagagccctgacaggcatcgccgtggagcaggacaagaacacccaggaggtgttcgcccaggtgaagcagatatataagacaccccctatcaaggacttcggcggctttaatttcagccagatcctgcccgatcccagcaagccctccaagaggtcctttatcgaggacctgctgtttaacaaggtgacactggccgacgccggctttatcaagcagtacggcgactgcctgggcgacatcgccgccagagatttgatctgcgcccagaagttcaatggcctgacagtgctgccccctctgctgacagatgagatgatcgcccagtacacaagcgccctgctggccggcaccatcacctccggttggacatttggcgccggcgccgccctgcagatcccttttgccatgcagatggcctacagattcaacggcatcggcgtgacccagaacgtgctgtacgagaaccagaagctgatcgccaatcagttcaatagcgccatcggcaagatccaggacagcctgagctccaccgccagcgccctgggaaagctgcaggacgtggtgaatcagaatgcccaggccctgaacaccctggtgaagcagctgtcctccaattttggcgccatctccagcgtgctgaatgacatcctgtccaggctggacaaggtggaggccgaggtgcagatcgacagactgatcacaggcaggctgcagagcctgcagacctacgtgacccagcagctgatcagagccgccgagatcagggcctccgccaacctggccgccaccaagatgagcgagtgcgtgctgggccagtccaagagagtggatttctgtggcaagggctaccacctgatgagcttccctcagagcgcccctcacggcgtggtgttcctgcacgtgacctacgtgcccgcccaggagaagaattttaccaccgcccctgccatctgccacgatggcaaggcccacttccccagagagggcgtgtttgtgtccaatggcacacactggtttgtgacccagaggaacttctacgagccccagatcatcaccacagacaatacatttgtgtccggcaattgcgacgtggtcattggcatcgtgaataataccgtgtacgaccctctgcagcctgagctggatagctttaaggaggagctggataagtactttaagaaccacaccagccccgacgtggatctgggcgacatttccggcatcaacgccagcgtggtgaacatccagaaggagatcgacagactcaacgaggtggccaagaacctgaatgagagcctgatcgacctgcaggagctgggcaagtacgagcagtacatcaagtggcctatcgacagcggcggcggcggctctggatgtcctggactgcccggcgcccccggacctaaaggagaggctggcaccaatggcaagagaggcgagaggggcccccctggacccccaggaaaggctggaccccccggaccaaacggcgcccctggagaacctacctcctttgagcaccaccaccaccatcaccaccactaagcggccgc

SEQ ID NO: 5 - DNA, 15 bp, synthetic construct
  source 1..15: mol_type = other DNA
cagaccattctgcgc

SEQ ID NO: 6 - protein, 6 aa, synthetic construct
QTILRS

SEQ ID NO: 7 - DNA, 18 bp, synthetic construct
  source 1..18: mol_type = other DNA
tccggaggcggcggcagc

SEQ ID NO: 8 - DNA, 24 bp, synthetic construct
  source 1..24: mol_type = other DNA
caccaccaccaccatcaccaccac

SEQ ID NO: 9 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 673 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 11 - protein, 588 aa, synthetic construct
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 4 aa, synthetic construct
RRAR

SEQ ID NO: 13 - protein, 4 aa, synthetic construct
GSAS

SEQ ID NO: 14 - protein, 10 aa, synthetic construct
QTNSPRRRAR

SEQ ID NO: 15 - protein, 5 aa, synthetic construct
QTILR

SEQ ID NO: 16 - protein, 1197 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTILRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 17 - DNA, 3915 bp, synthetic construct
  source 1..3915: mol_type = other DNA
gaattccccagtggctgccatgggcgctggcgctacaggcagggctatggatggccccagactgctgctgctgctcctgctgggcgtgtccctgggcggagctagatcttcccagtgtgtgaacttcacaaacaggacacagctgcctagcgcctacaccaattccttcaccagaggcgtgtactaccctgacaaggtgttcagaagcagcgtgctgcacagcacacaggacctgttcctgcccttcttctccaatgtgacatggttccacgccatctccggcaccaatggcacaaagagattcgccaaccctgtgctgcccttcaatgacggcgtgtacttcgccagcaccgagaagagcaacatcatcaggggctggatcttcggcaccacactggacagcaagacacagtccctgctgatcgtgaacaatgccaccaacgtggtgatcaaggtgtgtgagttccagttctgtaattaccccttcctgggcgtgtaccacaagaataacaagtcctggatggagtccgagttcagagtgtactcctccgccaataactgtacattcgagtacgtgagccagcccttcctgatggatctggagggcaagcagggcaacttcaagaacctgagcgagttcgtgttcaagaacatcgatggctacttcaagatatatagcaagcacacccccatcaatctggtgaggggcctgcctcagggcttcagcgccctggagcccctggtggatctgcccatcggcatcaacatcaccagattccagacactgcacatctcctacctgacacctggcgacagcagcagcggctggacagccggagccgctgcttactacgtgggctacctgcagcctaggaccttcctgctgaagtacaatgagaatggcaccatcacagacgccgtggattgcgccctggatcccctgagcgagacaaagtgcaccctgaagagcttcaccgtggagaagggcatctaccagacctccaacttcagagtgcagcctaccgagagcatcgtgaggttccccaacatcacaaacctgtgtcccttcggcgaggtgttcaatgccaccaggttcgcctccgtgtacgcctggaatagaaagagaatcagcaattgtgtggccgattacagcgtgctgtacaacagcgccagcttctccacattcaagtgctacggcgtgtcccctacaaagctgaatgacctgtgcttcacaaacgtgtacgccgactccttcgtgatcagaggcgacgaggtgagacagatcgcccctggccagacaggcaacatcgccgactacaattacaagctgcccgatgacttcacaggctgcgtgatcgcctggaatagcaataacctggatagcaaggtgggcggcaactacaattacctgtacagactgttcagaaagagcaatctgaagcccttcgagagagatatcagcacagagatatatcaggccggcagcaccccctgcaatggcgtgaagggcttcaactgctacttccctctgcagagctacggcttccagcccacctacggcgtgggctaccagccctacagagtggtggtgctgtccttcgagctgctgcacgcccccgccacagtgtgtggccctaagaagagcacaaacctggtgaagaataagtgcgtgaacttcaacttcaatggcctgaccggcacaggcgtgctgaccgagagcaataagaagttcctgcccttccagcagttcggcagggatatcgatgatacaaccgatgccgtgagggacccccagaccctggagatcctggatatcacaccctgcagcttcggcggcgtgtccgtgatcacccctggcacaaatacaagcaaccaggtggccgtgctgtaccagggcgtgaactgcaccgaggtgcccgtggccatccacgccgatcagctgacccccacctggagagtgtacagcacaggcagcaacgtgttccagacaagagccggctgcctgatcggcgccgagtacgtgaataacagctacgagtgcgatatccctatcggcgccggcatctgtgccagctaccagacccagaccaacagccacggctccgcctcctccgtggcctctcagagcatcatcgcctacacaatgtccctgggcgtggagaactccgtggcctacagcaataattccatcgccatccccacaaacttcaccatcagcgtgacaaccgagatcctgcccgtgagcatgacaaagacaagcgtggattgcacaatgtacatctgtggcgattccaccgagtgcagcaatctgctgctgcagtacggcagcttctgtatccagctgaatagggccctgaccggcatcgccgtggagcaggataagaatacccaggaggtgttcgcccaggtgaagcagatatataagaccccccccatcaaggacttcggcggcttcaacttcagccagatcctgcccgatcctagcaagccttccaagagaagcttcatcgaggacctgctgttcaacaaggtgacactggccgacgccggcttcatcaagcagtacggcgactgcctgggcgatatcgccgccagagacctgatctgtgcccagaagttcaacggcctgaccgtgctgccccccctgctgacagatgagatgatcgcccagtacacaagcgccctgctggccggcacaatcaccagcggctggaccttcggcgccggcgctgctctgcagatccccttcgccatgcagatggcctacaggttcaacggcatcggcgtgacccagaatgtgctgtacgagaaccagaagctgatcgccaaccagttcaactccgccatcggcaagatccaggattccctgagcagcacagcctccgccctgggcaagctgcaggacgtggtgaaccagaacgcccaggccctgaataccctggtgaagcagctgtcctccaacttcggcgccatctccagcgtgctgaacgacatcctggccagactggacaaggtggaggccgaggtgcagatcgacaggctgatcaccggcaggctgcagtccctgcagacctacgtgacacagcagctgatcagagccgccgagatcagagcctccgccaacctggccgccatcaagatgagcgagtgtgtgctgggccagtccaagagggtggacttctgtggcaagggctaccacctgatgagcttcccccagagcgcccctcacggcgtggtgttcctgcacgtgacctacgtgcccgcccaggagaagaacttcacaaccgcccccgccatctgccacgacggcaaggctcacttccctagagagggcgtgttcgtgagcaatggcacacactggttcgtgacccagaggaacttctacgagccccagatcatcacaacacacaatacattcgtgagcggcaattgtgatgtggtgatcggcatcgtgaacaacacagtgtacgatcctctgcagcctgagctggacagcttcaaggaggagctggacaagtacttcaagaatcacacatcccccgatgtggacctgggcgatattagcggcatcaacgccagcgtggtgaacatccagaaggagatcgataggctgaacgaggtggccaagaacctgaacgagtccctgatcgatctgcaggagctgggcaagtacgagcagtacatcaagtggccttccggaggcggcggcagcggatgccctggactgcctggagccccaggccctaagggcgaggctggaacaaatggcaagagaggcgagaggggccctcccggacctccaggaaaggccggacctcccggccctaatggcgcccctggagagccacagccctgcctggaggtgctgttccagggccctcaccaccaccaccatcaccaccactgagcggccgc

SEQ ID NO: 18 - protein, 1195 aa, synthetic construct
SQCVNFTNRTQLPSAYTNSFTRGVYYPDKVFRSSVLHSTQDLFLPFFSNVTWFHAISGTNGTKRFANPVLPFNDGVYFASTEKSNIIRGWIFGTTLDSKTQSLLIVNNATNVVIKVCEFQFCNYPFLGVYHKNNKSWMESEFRVYSSANNCTFEYVSQPFLMDLEGKQGNFKNLSEFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSHGSASSVASQSIIAYTMSLGVENSVAYSNNSIAIPTNFTISVTTEILPVSMTKTSVDCTMYICGDSTECSNLLLQYGSFCIQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 19 - protein, 1198 aa, synthetic construct
SQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 20 - protein, 1201 aa, synthetic construct
SQCVNFTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 21 - protein, 4 aa, synthetic construct
SGAG

SEQ ID NO: 22 - protein, 12 aa, synthetic construct
MFVFLVLLPLVS

SEQ ID NO: 23 - protein, 28 aa, synthetic construct
MGAGATGRAMDGPRLLLLLLLGVSLGGA

SEQ ID NO: 24 - protein, 8 aa, synthetic construct
LANSPVAA

SEQ ID NO: 25 - protein, 45 aa, Homo sapiens
GCPGLPGAPGPKGEAGTNGKRGERGPPGPPGKAGPPGPNGAPGEP

SEQ ID NO: 26 - protein, 48 aa, Homo sapiens
GCPGLPGAPGPKGEAGTNGKRGERGPPGPPGKAGPPGPNGAPGEPQPC

SEQ ID NO: 27 - protein, 70 aa, Homo sapiens
LQAADTCPEVKMVGLEGSDKLTILRGCPGLPGAPGPKGEAGTNGKRGERGPPGPPGKAGPPGPNGAPGEP

SEQ ID NO: 28 - protein, 55 aa, Homo sapiens
GCPGLPGAPGPKGEAGTNGKRGERGPPGPPGKAGPPGPNGAPGEPQPCLTGPRTC

SEQ ID NO: 29 - protein, 218 aa, Homo sapiens
QPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 30 - protein, 25 aa, Homo sapiens
LQAADTCPEVKMVGLEGSDKLTILR

SEQ ID NO: 31 - protein, 18 aa, synthetic construct
SGGGGSSGGGGSSGGGGS

SEQ ID NO: 32 - protein, 30 aa, synthetic construct
SGGGGSSGGGGSSGGGGSSGGGGSSGGGGS

SEQ ID NO: 33 - protein, 57 aa, synthetic construct
RDCDPPGNPVHGYFEGNNFTLGSTISYYCEDRYYLVGVQEQQCVDGEWSSALPVCKL

SEQ ID NO: 34 - protein, 7 aa, Tobacco etch virus
ENLYFQG

SEQ ID NO: 35 - protein, 8 aa, Human rhinovirus
LEVLFQGP

SEQ ID NO: 36 - protein, 16 aa, synthetic construct
LEVLFQGBHHHHHHHH

SEQ ID NO: 37 - protein, 10 aa, Homo sapiens
QPCLTGPRTC

SEQ ID NO: 38 - protein, 31 aa, Homo sapiens
LQAADTCPEVKMVGLEGSDKLTILRGCPGLP

SEQ ID NO: 39 - protein, 45 aa, synthetic construct
GAPGLPGAPGPKGEAGTNGKRGERGPPGPPGKAGPPGPNGAPGEP

SEQ ID NO: 40 - protein, 68 aa, synthetic construct
IDSGGGGSGCPGLPGAPGPKGEAGTNGKRGERGPPGPPGKAGPPGPNGAPGEPQPCTSFEHHHHHHHH

SEQ ID NO: 41 - protein, 65 aa, synthetic construct
IDSGGGGSGCPGLPGAPGPKGEAGTNGKRGERGPPGPPGKAGPPGPNGAPGEPTSFEHHHHHHHH

SEQ ID NO: 42 - protein, 30 aa, synthetic construct
  REGION 2..3: note = wherein X is C, L, A,P, E, T or K
  REGION 5..6: note = wherein X is C, L, A,P, E, T or K
  REGION 8..9: note = wherein X is C, L, A,P, E, T or K
  REGION 11..12: note = wherein X is C, L, A,P, E, T or K
  REGION 14..15: note = wherein X is C, L, A,P, E, T or K
  REGION 17..18: note = wherein X is C, L, A,P, E, T or K
  REGION 20..21: note = wherein X is C, L, A,P, E, T or K
  REGION 23..24: note = wherein X is C, L, A,P, E, T or K
  REGION 26..27: note = wherein X is C, L, A,P, E, T or K
  REGION 29..30: note = wherein X is C, L, A,P, E, T or K
GXXGXXGXXGXXGXXGXXGXXGXXGXXGXX

SEQ ID NO: 43 - protein, 288 aa, Homo sapiens
LQAADTCPEVKMVGLEGSDKLTILRGCPGLPGAPGPKGEAGTNGKRGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 44 - DNA, 3921 bp, synthetic construct
  source 1..3921: mol_type = other DNA
gaattcaccggtcgccgccatgggcgccggcgccaccggccgcgccatggacggcccccgcctgctgctgctgctgctgctgggcgtgagcctgggcggcgccagatcttcccagtgcgtgaacctgaggaccagaacccagctgccccccgcctacacaaatagcttcaccagaggcgtgtactaccctgacaaggtgtttagaagcagcgtgctgcacagcacccaggacctgtttctgcctttctttagcaacgtgacctggttccacgccatccacgtgagcggcacaaatggcaccaagagattcgataatcccgtgctgccttttaacgacggcgtgtacttcgcctccaccgagaagagcaacatcatcaggggctggatcttcggcacaaccctggatagcaagacccagtccctgctgatcgtgaataacgccaccaacgtggtcattaaggtgtgcgagtttcagttctgcaacgaccctttcctgggcgtgtactatcacaagaacaataagagctggatggagagcgtgtactcctccgccaacaattgcacatttgagtacgtgtcccagcctttcctgatggatctggagggcaagcagggcaacttcaagaacctgagagagttcgtgttcaagaacatcgatggctacttcaagatatattccaagcacacccctatcaatctggtgagagatttgccccagggcttcagcgccctggagcctctggtggatctgcctatcggcatcaacatcaccaggttccagaccctgctggccctgcacaggagctacctgacccctggcgattcctccagcggctggaccgccggagccgctgcttactacgtgggctacctgcagcccaggacattcctgctgaagtacaatgagaatggcaccatcaccgatgccgtggactgcgccctggaccccctgtctgagaccaagtgtaccctgaagtccttcaccgtggagaagggcatctaccagacatccaactttagggtgcagcccaccgagagcatcgtgaggtttcccaacatcaccaatctgtgccctttcggcgaggtgttcaatgccaccaggtttgccagcgtgtacgcctggaacagaaagagaatcagcaactgtgtggccgactacagcgtgctgtacaactccgccagcttctccacctttaagtgctacggcgtgtcccctacaaagctgaatgacctgtgtttcacaaacgtgtacgccgactccttcgtgatcagaggcgacgaggtgagacagatcgcccctggccagaccggcaagatcgccgactacaactacaagctgcccgacgacttcaccggctgtgtgatcgcctggaatagcaacaatctggactccaaggtgggcggcaattacaactacaggtacaggctgttcaggaagtccaatctgaagccttttgagagggacatcagcacagagatatatcaggccggctccaagccctgcaacggcgtggagggctttaattgttacttccccctgcagtcctacggcttccagcccaccaatggcgtgggctaccagccctacagagtggtggtgctgtcctttgagctgctgcacgcccctgccacagtgtgtggccccaagaagagcaccaatctggtgaagaataagtgcgtgaactttaactttaacggcctgacaggcaccggcgtgctgaccgagagcaataagaagtttctgcccttccagcagtttggcagagacatcgccgatacaaccgatgccgtcagggacccccagaccctggagatcctggatatcacaccttgctccttcggcggcgtgtccgtgatcacccctggcaccaacacctccaaccaggtggccgtgctgtaccagggcgtgaattgtaccgaggtgcccgtggccatccacgccgatcagctgacccctacatggagagtgtacagcacaggcagcaacgtgtttcagacaagagccggctgtctgatcggcgccgagcacgtgaacaactcctacgagtgcgacatccctatcggcgccggcatctgtgcctcctaccagacccagacaaattccaggggctccgccagcagcgtggcctctcagagcatcatcgcctacaccatgtccctgggcgccgagaacagcgtggcctactccaacaacagcatcgccatccccaccaattttaccatcagcgtgaccaccgagatcctgcccgtgagcatgaccaagaccagcgtggattgcaccatgtacatctgtggcgactccacagagtgctccaatctgctgctgcagtacggctccttctgtacacagctgaatagagccctgaccggcatcgccgtggagcaggacaagaacacacaggaggtgttcgcccaggtgaagcagatatataagaccccccccatcaaggactttggcggcttcaatttttcccagatcctgcccgatcctagcaagcccagcaagaggagcttcatcgaggacctgctgtttaacaaggtgacactggccgacgccggcttcatcaagcagtacggcgattgcctgggcgacatcgccgccagggacctgatctgcgcccagaagttcaacggcctgaccgtgctgcctcctctgctgacagatgagatgatcgcccagtacacctccgccctgctggccggaaccatcacatccggctggaccttcggcgccggcgctgctctgcagatcccttttgccatgcagatggcctacagattcaacggcatcggcgtgacacagaacgtgctgtacgagaatcagaagctgatcgccaatcagttcaatagcgccatcggcaagatccaggatagcctgagctccaccgccagcgccctgggaaagctgcagaatgtggtgaaccagaatgcccaggccctgaacaccctggtgaagcagctgagctccaactttggcgccatcagctccgtgctgaacgatatcctgagcagactggacaaggtggaggccgaggtgcagatcgacaggctgatcaccggcagactgcagagcctgcagacctacgtgacacagcagctgatcagggccgccgagatcagggccagcgccaacctggccgccaccaagatgagcgagtgtgtgctgggccagtccaagagggtggacttctgcggcaagggctaccacctgatgtccttccctcagtccgccccccacggcgtggtgttcctgcacgtgacctacgtgcccgcccaggagaagaattttacaaccgcccctgccatctgccacgatggcaaggcccacttccctagagagggcgtgtttgtgagcaatggcacacactggtttgtgacacagagaaacttttacgagccccagatcatcacaaccgacaataccttcgtgagcggcaattgtgacgtggtcattggcatcgtgaacaacacagtgtacgatcctctgcagcccgagctggattcctttaaggaggagctggacaagtacttcaagaatcacacctcccctgacgtggatctgggcgacattagcggcatcaatgccagcgtggtgaatatccagaaggagatcgacagactgaacgaggtggccaagaacctgaatgagtccctgatcgacctgcaggagctgggcaagtacgagcagtacatcaagtggccctccggaggcggcggctctggatgccctggactgccaggcgcccctggacctaagggcgaggctggaaccaatggcaagaggggcgagaggggcccccctggaccacctggaaaggccggacctcccggcccaaacggcgctcctggagagcctaaggacctggaggtgctgttccagggccctcaccaccaccaccatcaccaccactgagcggccgc

SEQ ID NO: 45 - protein, 1303 aa, synthetic construct
NSPVAAMGAGATGRAMDGPRLLLLLLLGVSLGGARSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGGGGSGCPGLPGAPGPKGEAGTNGKRGERGPPGPPGKAGPPGPNGAPGEPKDLEVLFQGPHHHHHHHH

SEQ ID NO: 46 - DNA, 3921 bp, synthetic construct
  source 1..3921: mol_type = other DNA
gaattcaccggtcgccgccatgggcgccggcgccaccggccgcgccatggacggcccccgcctgctgctgctgctgctgctgggcgtgagcctgggcggcgccagatcttcccagtgcgtgaacctgaccacaaggacacagctgccccctgcctacaccaatagcttcaccagaggcgtgtactaccccgataaggtgttcaggagcagcgtgctgcacagcacccaggatctgttcctgcccttcttctccaacgtgacatggttccacgtgatcagcggcaccaacggcacaaagagattcgataatcccgtgctgcccttcaacgacggcgtgtacttcgccagcatcgagaagtccaatatcatcagaggctggatcttcggcacaaccctggactccaagacacagagcctgctgatcgtgaacaacgccacaaacgtggtgatcaaggtgtgtgagttccagttctgcaacgaccccttcctggaccacaagaataacaagagctggatggagtccgagttcagagtgtacagcagcgccaataactgcaccttcgagtacgtgagccagcccttcctgatggacctggagggcaagcagggcaacttcaagaacctgagggagttcgtgttcaagaacatcgacggctacttcaagatatattccaagcacacccccatcatcgtggagcccgagagagacttacctcagggcttcagcgccctggagcctctggtggacctgcccatcggcatcaatatcaccagattccagaccctgctggccctgcacagatcctacctgacccccggcgacagcagcagcggctggacagctggcgccgctgcttactacgtgggctacctgcagcctagaaccttcctgctgaagtacaacgagaatggcaccatcaccgatgccgtggactgtgccctggaccctctgtccgagaccaagtgtacactgaagtccttcacagtggagaagggcatctaccagaccagcaacttcagagtgcagcccaccgagtccatcgtgagattccccaatatcaccaatctgtgtcccttcgacgaggtgttcaatgccaccaggttcgccagcgtgtacgcctggaatagaaagaggatcagcaactgcgtggccgactacagcgtgctgtacaatctggcccccttcttcaccttcaagtgctacggcgtgtcccctaccaagctgaacgacctgtgcttcaccaatgtgtacgccgacagcttcgtgatcagaggcgacgaggtgagacagatcgcccccggccagacaggcaatatcgccgactacaactacaagctgcctgacgacttcacaggctgtgtgatcgcctggaactccaacaagctggactccaaagtgtccggcaactacaattacctgtacaggctgttcagaaagtccaacctgaagcccttcgagagggacatctccacagagatatatcaggccggcaacaagccttgcaacggcgtggccggcttcaactgttacttccccctgaggagctacagcttcaggcccacatacggcgtgggccaccagccctacagggtggtggtgctgtccttcgagctgctgcacgcccccgccaccgtgtgtggacctaagaagagcaccaacctggtgaagaacaagtgtgtgaacttcaacttcaatggcctgaagggcacaggcgtgctgacagagtccaataagaagttcctgcccttccagcagttcggcagagatatcgccgacaccacagatgccgtgagagacccccagacactggagatcctggacatcaccccctgcagcttcggcggcgtgtccgtgatcacccctggcacaaacacctccaatcaggtggccgtgctgtaccagggcgtgaattgtacagaggtgcccgtggccatccacgccgaccagctgacccctacctggagagtgtactccacaggcagcaatgtgttccagaccagggccggctgcctgatcggcgctgagtacgtgaataacagctacgagtgtgatatccctatcggcgccggcatctgcgcctcctaccagacacagaccaagagccacggcagcgcctccagcgtggcctctcagtccatcatcgcctacacaatgtccctgggcgccgagaacagcgtggcctacagcaataacagcatcgccatccccaccaacttcaccatctccgtgacaaccgagatcctgcctgtgtccatgaccaagacatccgtggattgtacaatgtacatctgcggcgattccaccgagtgttccaacctgctgctgcagtacggctccttctgtacccagctgaagagagccctgacaggcatcgccgtggagcaggataagaacacccaggaggtgttcgcccaggtgaagcagatatataagacaccccctatcaagtacttcggcggcttcaacttcagccagatcctgcccgacccttccaagcccagcaagaggagcttcatcgaggacctgctgttcaataaggtgaccctggccgatgccggcttcatcaagcagtacggcgattgtctgggcgatatcgccgccagagacttaatctgcgcccagaagttcaagggcctgacagtgctgcctcctctgctgaccgatgagatgatcgcccagtacacctccgccctgctggccggaaccatcaccagcggctggaccttcggcgccggcgctgctctgcagatccccttcgccatgcagatggcctacaggttcaatggcatcggcgtgacccagaacgtgctgtacgagaatcagaagctgatcgccaaccagttcaattccgccatcggcaagatccaggacagcctgagcagcaccgcctccgccctgggaaagctgcaggatgtggtgaatcacaatgcccaggccctgaatacactggtgaagcagctgtcctccaagttcggcgccatcagctccgtgctgaatgatatcttcagcagactggataaggtggaggccgaggtgcagatcgacaggctgatcacaggcagactgcagtccctgcagacatacgtgacacagcagctgatcagagccgccgagatcagggcctccgccaatctggccgccacaaagatgtccgagtgtgtgctgggccagagcaagagagtggacttctgtggcaagggctaccacctgatgtccttccctcagtccgcccctcacggcgtggtgttcctgcacgtgacatacgtgcccgcccaggagaagaacttcacaaccgcccccgccatctgtcacgacggcaaggcccacttccctagggagggcgtgttcgtgagcaacggcacacactggttcgtgacccagaggaacttctacgagcctcagatcatcaccacagataatacattcgtgtccggcaattgcgacgtggtgatcggcatcgtgaacaataccgtgtacgatcccctgcagcccgagctggatagcttcaaggaggagctggacaagtacttcaagaatcacacatcccctgacgtggatctgggcgatattagcggcatcaatgccagcgtggtgaatatccagaaggagatcgacagactgaacgaggtggccaagaatctgaacgagtccctgatcgacctgcaggagctgggcaagtacgagcagtacatcaagtggccctccggaggcggcggctctggatgccctggactgcctggcgcccccggacctaagggagaggctggaacaaatggcaagagaggcgagaggggccctcctggcccccctggaaaggctggacctcctggcccaaatggcgcccccggtgagcctaaggatctggaggtgctgttccagggccctcaccaccaccaccatcaccaccactgagcggccgc

SEQ ID NO: 47 - protein, 1303 aa, synthetic construct
NSPVAAMGAGATGRAMDGPRLLLLLLLGVSLGGARSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVEPERDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGGGGSGCPGLPGAPGPKGEAGTNGKRGERGPPGPPGKAGPPGPNGAPGEPKDLEVLFQGPHHHHHHHH